SEQUENCE LISTING (44 sequences)

SEQ ID NO: 1 - protein, 269 aa, Artificial
  VyPAL2 core domain
DSIGTRWAVLIAGSKGYHNYRHQADVCHMYQILRKGGVKDENIIVFMYDDIAYNESNPFPGIIINKPGGENVYKGVPKDYTGEDINNVNFLAAILGNKSAIIGGSGKVLDTSPNDHIFIYYADHGAPGKIGMPSKPYLYADDLVDTLKQKAATGTYKSMVFYVEACNAGSMFEGLLPEGTNIYAMAASNSTEGSWITYCPGTPDFPPEFDVCLGDLWSITFLEDCDAHNLRTETVHQQFELVKKKIAYASTVSQYGDIPISKDSLSVYM

SEQ ID NO: 2 - protein, 281 aa, Artificial
  butelase-1 active fragment
VEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPAN

SEQ ID NO: 3 - protein, 483 aa, Viola yedoensis
MQLFAAGVILFFLLALSGTIAGGLDVDSLQLPSEAAKFFHNDNSTNDDDSIGTRWAVLIAGSKGYHNYRHQADVCHMYQILRKGGVKDENIIVFMYDDIAYNESNPFPGIIINKPGGENVYKGVPKDYTGEDINNVNFLAAILGNKSAIIGGSGKVLDTSPNDHIFIYYADHGAPGKIGMPSKPYLYADDLVDTLKQKAATGTYKSMVFYVEACNAGSMFEGLLPEGTNIYAMAASNSTEGSWITYCPGTPDFPPEFDVCLGDLWSITFLEDCDAHNLRTETVHQQFELVKKKIAYASTVSQYGDIPISKDSLSVYMGTDPANDNRTFVDENSLRPPLKVIHQHDADLYHIWCKYNMAPEGSSKKIEAQKQLLELMSHRAHVDNSITLIGKLLFGVNKASKVLNTVRPVGQPLVDDWQCLKAMIRTFETHCGSLSEYGMKHTLSFANMCNAGIQKEQLAEAAAQACVTFPSNPYSSLAEGFSA

SEQ ID NO: 4 - protein, 482 aa, Clitoria ternatea
MKNPLAILFLIATVVAVVSGIRDDFLRLPSQASKFFQADDNVEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPANDNNTFTDKNSLGTPRKAVSQRDADLIHYWEKYRRAPEGSSRKAEAKKQLREVMAHRMHIDNSVKHIGKLLFGIEKGHKMLNNVRPAGLPVVDDWDCFKTLIRTFETHCGSLSEYGMKHMRSFANLCNAGIRKEQMAEASAQACVSIPDNPWSSLHAGFSV

SEQ ID NO: 5 - protein, 6 aa, Artificial
  MLA motif
KKIAYA

SEQ ID NO: 6 - protein, 6 aa, Artificial
  MLA motif
NKIAYA

SEQ ID NO: 7 - protein, 9 aa, Artificial
  Peptide
KKLAVINHV

SEQ ID NO: 8 - protein, 7 aa, Artificial
  Peptide
GIGGIKA

SEQ ID NO: 9 - protein, 6 aa, Artificial
  Peptide
YKANGL

SEQ ID NO: 10 - protein, 7 aa, Artificial
  Peptide
GFGGIKA

SEQ ID NO: 11 - protein, 9 aa, Artificial
  Peptide
KKLAVINGF

SEQ ID NO: 12 - protein, 6 aa, Artificial
  Peptide
GRANGI

SEQ ID NO: 13 - protein, 22 aa, Artificial
  Peptide
  MISC_FEATURE (9)..(22): D-amino acid
GIGGFKGGKLAKLAKKLAKLAK

SEQ ID NO: 14 - protein, 10 aa, Artificial
  Peptide
  MISC_FEATURE (6)..(6): modified with aminooxy functional group
GFLGVKANHV

SEQ ID NO: 15 - protein, 16 aa, Artificial
  Peptide
GISTKSIPPISYRDGL

SEQ ID NO: 16 - protein, 16 aa, Artificial
  Peptide
GISTKSIPPISYRDDL

SEQ ID NO: 17 - protein, 16 aa, Artificial
  Peptide
GISTKSIPPISYRDAL

SEQ ID NO: 18 - protein, 16 aa, Artificial
  Peptide
GISTKSIPPISYRDLL

SEQ ID NO: 19 - protein, 16 aa, Artificial
  Peptide
GISTKSIPPISYRDSL

SEQ ID NO: 20 - protein, 16 aa, Artificial
  Peptide
GISTKSIPPISYRDRL

SEQ ID NO: 21 - protein, 16 aa, Artificial
  Peptide
GISTKSIPPISYRDKL

SEQ ID NO: 22 - protein, 16 aa, Artificial
  Peptide
GISTKSIPPISYRDQL

SEQ ID NO: 23 - protein, 16 aa, Artificial
  Peptide
GISTKSIPPISYRDEL

SEQ ID NO: 24 - protein, 16 aa, Artificial
  Peptide
GISTKSIPPISYRNGL

SEQ ID NO: 25 - protein, 7 aa, Artificial
  Peptide
GIGGIRK

SEQ ID NO: 26 - protein, 20 aa, Artificial
  Peptide
QRLGNQWAVGHLMGSGSDSL

SEQ ID NO: 27 - protein, 17 aa, Artificial
  Peptide
IHGHHIISVGGSGSDSL

SEQ ID NO: 28 - protein, 19 aa, Artificial
  Peptide
VPWMEPAYQRFLGSGSDSL

SEQ ID NO: 29 - protein, 19 aa, Artificial
  Peptide
YHWYGYTPQKVIGSGSDSL

SEQ ID NO: 30 - protein, 19 aa, Artificial
  Peptide
CMYIEALDKYACGSGSDSL

SEQ ID NO: 31 - protein, 15 aa, Artificial
  Peptide
CRGDRGDCGSGSDSL

SEQ ID NO: 32 - protein, 6 aa, Artificial
  Peptide
GRADGI

SEQ ID NO: 33 - protein, 5 aa, Artificial
  Peptide
GIAAK

SEQ ID NO: 34 - protein, 6 aa, Artificial
  Peptide
YKANGL

SEQ ID NO: 35 - protein, 80 aa, Artificial
  ZEGFR
GFGSSLQVDNKFNKEMWAAWEEIRNLPNLNGWQMTAFIASLVDDPSQSANLLAEAKKLNDAQAPKVDGSGSNHVHHHHHH

SEQ ID NO: 36 - protein, 90 aa, Artificial
  Ubiquitin tagged
GGSGSGSQIFVKTLTGKTITLEVEPSDTIENVKAKIQDKEGIPPDQQRLIFAGKQLEDGRTLSDYNIQKESTLHLVLRLRGGNGIGGIRK

SEQ ID NO: 37 - protein, 76 aa, Artificial
  Affibody
GRANGFGSSLQVDNKFNKEMWAAWEEIRNLPNLNGWQMTAFIASLVDDPSQSANLLAEAKKLNDAQAPKVDGSGSN

SEQ ID NO: 38 - protein, 78 aa, Artificial
  ZEGFR
GSSHHHHHHLQVDNKFNKEMWAAWEEIRNLPNLNGWQMTAFIASLVDDPSQSANLLAEAKKLNDAQAPKVDGSGSNGL

SEQ ID NO: 39 - protein, 244 aa, Artificial
  sfGFP-DSL-His6x
GIVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDSLHHHHHH

SEQ ID NO: 40 - protein, 81 aa, Artificial
  Affibody
MKKGSSHHHHHHLQVDNKFNKEMWAAWEEIRNLPNLNGWQMTAFIASLVDDPSQSANLLAEAKKLNDAQAPKVDGSGSDSL

SEQ ID NO: 41 - protein, 81 aa, Artificial
  Affibody
MKKGSSHHHHHHLQVDNKFNKEMWAAWEEIRNLPNLNGWQMTAFIASLVDDPSQSANLLAEAKKLNDAQAPKVDGSGSDGL

SEQ ID NO: 42 - protein, 239 aa, Artificial
  sfGFP-His6x
GVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGINSLHHHHHH

SEQ ID NO: 43 - protein, 79 aa, Artificial
  ZEGFR
IGSSHHHHHHLQVDNKFNKEMWAAWEEIRNLPNLNGWQMTAFIASLVDDPSQSANLLAEAKKLNDAQAPKVDGSGSDSL

SEQ ID NO: 44 - protein, 299 aa, Artificial
  OaAEP1b functional fragment
SVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWAYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDAD

(19) United States
(12) Patent Application Publication
(10) Pub. No.: US 2024/0117402 A1
(43) Pub. Date: Apr. 11, 2024

(54) METHODS FOR (POLY) PEPTIDE TANDEM LIGATION AND CYCLIZATION

(71) Applicant: Nanyang Technological University, Singapore (SG)

(72) Inventors: Chuan Fa Liu, Singapore (SG); Dingpeng Zhang, Singapore (SG); Zhen Wang, Singapore (SG); James P. Tam, Singapore (SG)

(21) Appl. No.: 18/264,843

(22) PCT Filed: Feb. 10, 2022
(86) PCT No.: PCT/SG2022/050069

§ 371 Date: Aug. 9, 2023

(30) Foreign Application Priority Data
Feb. 10, 2021 (SG) 10202101440P

(51) Int. Cl. C12P 21/02; C12N 9/00

(52) U.S. Cl. CPC C12P 21/02; C12N 9/93

(57) ABSTRACT

The present invention relates to methods that employ enzymes having Asx-specific ligase and cyclase activity, namely butelase-1, VyPAL2 and OaAEPI b, as a means for engineering novel (poly) peptide theranostics. The differential substrate specificities and differential optimal pH of the Asx-specific ligase and cyclase are used to provide sufficient orthogonality for a tandem ligation and cyclization of proteins. Also encompassed are the corresponding uses.

### CROSS-REFERENCE TO RELATED APPLICATIONS

This application claims the benefit of priority of Singapore Patent Application No. 10202101440P filed Feb. 10, 2021, the content of which being hereby incorporated by reference in its entirety for all purposes.

### FIELD OF THE INVENTION

The present invention lies in the technical field of enzymatic (poly)peptide ligation and specifically relates to methods that employ enzymes having Asx-specific ligase and cyclase activity as a means for engineering novel (poly)peptide theranostics. Further encompassed are the corresponding uses.

### BACKGROUND OF THE INVENTION

Combining therapy with specific diagnostic information of a disease target, the concept of theranostics promises to bring optimized efficacy and safety of precision medicine [1-3]. This has stimulated tremendous interest in developing theranostic agents for cancer treatment [4, 5]. Besides the use of nanomedicine platforms for theranostics development [6-9], a molecular-based approach is through the attachment of imaging agents and cytotoxic drugs to cancer-targeting proteins and antibodies [10, 11]. In particular, small protein ligands such as antibody fragments and mimetics offer advantages of low production cost, good tissue penetration and easy maneuverability for designing end products with defined chemical composition. However, a major challenge in developing protein-based theranostic agents lies with the conjugation of the protein ligand with the imaging and treatment moieties [10]. Clearly, the conjugation strategy should be able to introduce at least two modifications onto a protein substrate. Although numerous chemical techniques have been developed for protein labeling [12, 13], no simple strategies are available that can allow two consecutive modifications to be done site-specifically on a straight recombinant protein.

With their high specificity and mild operating conditions, biosynthetic methods that modify proteins

through special recognition tags are attractive alternatives to chemical methods [14]. A main advantage of these tag-mediated protein labeling methods resides with the fact that the tags are themselves a peptide segment or protein domain and so can be genetically fused to the protein of interest (POI). Of most interest are those that are based on peptide ligases. Peptide ligases catalyze the formation of new peptide bonds between the ligation partners, which makes them particularly useful bioconjugation tools for protein-based theranostics. Notable examples of peptide ligases include subtiligase [15-19], sortase A [20-24] and butelase-1 [25-29] which are all tag-recognizing enzymes and can label proteins specifically at the terminal ends. Subtiligase is an artificially engineered ligase that uses an ester or thioester tag for protein labeling [15-19]. Sortase A requires a 5-residue tag LPETG and catalyzes transpeptidation at the Thr residue [20-24]. However, the use of the 5-residue tag notwithstanding, the enzymatic activity of sortase A is very low. Butelase-1 is a so-called peptidyl asparaginyl ligase or PAL and has been described in international patent publication WO 2015/163818 A1. So far, the most powerful peptide ligases are found in the PAL family and the most efficient PAL is butelase-1.

Structurally, butelase-1 is a member of the commonly known asparaginyl endopeptidase (AEP) or legumain family [30, 31]. Depending on pH or substrates, certain AEPs are also found to display PAL activities [32-42]. Butelase-1 is unique in that it functions almost as a pure PAL with no protease activity at weakly acidic to weakly basic pH. It has been shown to catalyze protein and peptide ligation with high specificity and efficiency [25-29]. Like all PALs, butelase-1 recognizes a short tripeptide tag such as NHV and cleaves the peptide bond at Asn to rejoin it with the amino terminal residue of another peptide. So only an Asn residue is left in the ligation product, making butelase-mediated ligation (BML) nearly traceless. This is in big contrast to most above-mentioned biosynthetic methods which leave a large “scar” in the modified protein [14]. Recently, VyPAL2, another plant legumain from the Viola Yedoensis family, was identified as a highly active PAL [42]. This PAL is also described in international patent publication WO 2020/226572 A1. Its catalytic efficiency was 274,325 M⁻¹ s⁻¹ in the cyclization

of a model peptide, making it one of the fastest PAL reported to date [42]. In addition, the proenzyme of VyPAL2 can be readily expressed in insect cells and be self-processed at acidic pH to yield the active enzyme [42]. These features make VyPAL2 a very attractive ligase for protein labeling [43]. Intriguingly,

there seem to be noticeable differences in substrate specificity between VyPAL2 and butelase-1. VyPAL2 has relatively low activity towards the tripeptide NHV which, on the other hand, is one of the preferred recognition motifs of butelase-1 [25, 42]. Also, a nucleophile peptide with a Phe at the P2″ position is a weak substrate for butelase-1 [25], but it is quite favored by VyPAL2 [42].

Several protein dual modification methods involving the use of peptide ligases have been reported [44-48]. For example, consecutive protein modifications were achieved chemoenzymatically by combining chemoselective conjugation and sortase A- or butelase-1 -mediated ligation [44-46]. Two sortases of different specificity were used to label a single protein at the N- and C-termini [49]. Butelase-1 was also used together with sortase A for protein dual labeling in a three-step scheme [46]. The two enzymes were also used for one-pot dual labeling of an antibody at the respective C-terminal ends of its light and

heavy chains [47]. These last two schemes are bio-orthogonal, taking advantage of the distinct substrate specificity of two completely different ligases. However, as discussed above, owing to its extremely slow kinetics and relatively long recognition tag, the use of sortase A has its inherent limitations. Recently, an interesting method was reported which allowed two consecutive ligation reactions on the same protein substrate from the C- to N-terminus direction [48]. However, it should be noted that this scheme is semi-orthogonal because it requires protection of the protein's N-terminal amine by a TEV recognition sequence during the first ligation step to avoid cyclization or self-ligation of the protein substrate [48].

### SUMMARY OF THE INVENTION

The inventors of the present invention found that the differential substrate specificities of butelase-1 and VyPAL2 provide sufficient orthogonality for a tandem ligation strategy for protein dual labeling. It was therefore possible to design a bio-orthogonal scheme using said two asparaginyl peptide ligases—butelase-1 and VyPAL2—which allows tandem asparaginyl ligation on the same protein in either N-to-C or C-to-N direction, leading to its dual labeling at the C- and N-terminal ends (FIG. 1). No protection on the protein substrate is required when performing the first ligation step, even though butelase-1 and VyPAL2 are both asparagine-specific. Thus, a distinct advantage of the bio-orthogonal ligation described herein is the use of mild enzymatic reactions under aqueous conditions, which are compatible with biologics, such as proteins, antibodies and live cells.

In addition to N- and C-terminal directed protein dual labeling, the herein described bio-orthogonal tandem ligation strategy can also be used to prepare a cycloprotein-drug conjugate or cPDC (FIG. 2). This involves the use of a synthetic intervening peptide designed to join the two termini of a protein. The peptide is trifunctional, containing an N-terminal GF-dipeptide nucleophile substrate for VyPAL, a C-terminal NHV tripeptide motif as the acyl substrate for butelase-1 and an internal aminooxy functionality for oxime conjugation, which would allow consecutive PAL-mediated ligation and cyclization as well as doxorubicin attachment (FIG. 2). With the expected thermal and metabolic stability of cycloproteins, the so-prepared cycloprotein conjugates are interesting theranostic candidates.

Because butelase-1 and VyPAL2 are the two most powerful ligases, such a bio-orthogonal tandem ligation strategy offers an ideal solution to the challenging problem of manufacturing protein-based theranostics and other biologics with unusual architecture and functionalities.

In addition, the PAL enzymes described and used herein can also catalyze peptide ligation at aspartyl peptide bonds albeit with significantly lower efficiency than at asparaginyl bonds. In spite of this, the efficiency of PAL-catalyzed aspartyl ligation is still much higher than sortase A-mediated ligation by at least two orders of magnitude. Because aspartyl peptide bonds are resistant to the PAL enzymes at around neutral pH—the pH for asparaginyl ligation, this orthogonality allows sequential aspartyl and asparaginyl ligations at different pH. This pH-controlled tandem ligation strategy also provides a useful solution to the challenging problem of manufacturing multi-functional protein theranostics.

In a first aspect, the present invention thus relates to a method for (poly)peptide tandem ligation, the method comprising the steps of:
(i) contacting a first (poly)peptide (A) having at its C-terminus a binding and ligation site for an asparaginyl ligase with a second (poly)peptide (B) to be ligated to said first (poly)peptide and a first asparaginyl ligase (C) under conditions that allow ligation of the second (poly)peptide to the C- or N-terminus of the first (poly)peptide to yield a modified first (poly)peptide;
(ii) contacting the modified first (poly)peptide obtained in step (i) with a third (poly)peptide (D) to be ligated to said modified first (poly)peptide and a second asparaginyl ligase (E) under conditions that allow ligation of the third (poly)peptide to the C- or N-terminus of the first (poly)peptide to yield a dually modified first (poly)peptide;
   wherein the first and second asparaginyl ligase are selected from VyPAL2 comprising or consisting of the amino acid sequence set forth in SEQ ID NO:1 and variants thereof that share at least 80% sequence identity with the amino acid sequence set forth in SEQ ID NO:1 over their entire length, and butelase-1 comprising or consisting of the amino acid sequence set forth in SEQ ID NO:2 and variants thereof that share at least 80% sequence identity with the amino acid sequence set forth in SEQ ID NO:2 over their entire length.

In various embodiments of this method, the second (poly)peptide has at its C-terminus a binding and ligation site for the first asparaginyl ligase and is ligated to the N-terminus of the first (poly)peptide by the first asparaginyl ligase. In such embodiments, the binding and ligation site for an asparaginyl ligase at the C-terminus of the first (poly)peptide may be for the second asparaginyl ligase and the third (poly)peptide can then be ligated to the C-terminus of the first (poly)peptide by the second asparaginyl ligase.

In various other embodiments, the second (poly)peptide is ligated to the C-terminus of the first (poly)peptide by the first asparaginyl ligase, wherein the binding and ligation site for an asparaginyl ligase at the C-terminus of the first (poly)peptide is for the first asparaginyl ligase. In such embodiments, the third (poly)peptide may have at its C-terminus a binding and ligation site for the second asparaginyl ligase and may be ligated to the N-terminus of the first (poly)peptide by the second asparaginyl ligase.

In various embodiments of the methods described herein, the first and second asparaginyl ligases are different and the first asparaginyl ligase is VyPAL2 or a variant thereof and the second asparaginyl ligase is butelase-1 or a variant thereof; or vice versa.

The binding and ligation site for VyPAL2 or a variant thereof may have, in various embodiments, the amino acid sequence (X)ₒNX³X⁴(X)ₚ, wherein X is any amino acid and o is an integer of at least 2, X 3 is G or S, and X 4 is a hydrophobic or aromatic amino acid, preferably selected from L, I, V, F, C, W, Y and M, preferably L or F, and p is 0 or an integer of 1 or more.

The binding and ligation site for butelase-1 or a variant thereof may have, in various embodiments, the amino acid sequence (X)ₒNX³X⁴(X)ₚ, wherein X is any amino acid and o is an integer of at least 2, X 3 is H, and X 4 is a hydrophobic or aromatic amino acid, preferably selected from L, I, V, F, C, W, Y and M, preferably V, and p is 0 or an integer of 1 or more.

In various embodiments,
(1) the first asparaginyl ligase is VyPAL2 or a variant thereof and said binding and ligation site for VyPAL2 or variant thereof is located at the C-terminus of the second (poly)peptide and the N-terminus of the first (poly)peptide has the amino acid sequence X¹F(X)_{q}, wherein X¹ can be any amino acid with the exception of Pro, X can be any amino acid, and q is 0 or an integer of 1 or more, preferably an integer of 1 or more, more preferably of at least 3, even more preferably of at least 5; or
(2) the first asparaginyl ligase is VyPAL2 or a variant thereof and said binding and ligation site for VyPAL2 or variant thereof is located at the C-terminus of the first (poly)peptide and the N-terminus of the second (poly)peptide has the amino acid sequence X¹F(X)_{q}, wherein X 1 can be any amino acid with the exception of Pro, X can be any amino acid, and q is 0 or an integer of 1 or more, preferably an integer of 1 or more, more preferably of at least 3, even more preferably of at least 5; or
(3) the second asparaginyl ligase is VyPAL2 or a variant thereof and said binding and ligation site for VyPAL2 or variant thereof is located at the C-terminus of the third (poly)peptide and the N-terminus of the first (poly)peptide has the amino acid sequence X¹F(X)_{q}, wherein X¹ can be any amino acid with the exception of Pro, X can be any amino acid, and q is 0 or an integer of 1 or more, preferably an integer of 1 or more, more preferably of at least 3, even more preferably of at least 5; or
(4) the second asparaginyl ligase is VyPAL2 or a variant thereof and said binding and ligation site for VyPAL2 or variant thereof is located at the C-terminus of the first (poly)peptide and the N-terminus of the third (poly)peptide has the amino acid sequence X¹F(X)_{q}, wherein X¹ can be any amino acid with the exception of Pro, X can be any amino acid, and q is 0 or an integer of 1 or more, preferably an integer of 1 or more, more preferably of at least 3, even more preferably of at least 5.

In various embodiments,
(1) the first asparaginyl ligase is butelase-1 or a variant thereof and said binding and ligation site for butelase-1 or variant thereof is located at the C-terminus of the second (poly)peptide and the N-terminus of the first (poly)peptide has the amino acid sequence X 1 X 2 (X) q with X¹ being G or H and X² being L, V or I, X being any amino acid, and q being 0 or an integer of 1 or more, preferably an integer of 1 or more, more preferably of at least 3, even more preferably of at least 5; or
(2) the first asparaginyl ligase is butelase-1 or a variant thereof and said binding and ligation site for butelase-1 or variant thereof is located at the C-terminus of the first (poly)peptide and the N-terminus of the second (poly)peptide has the amino acid sequence X¹X²(X)_{q} with X¹ being G or H and X² being L, V or I, X being any amino acid, and q being 0 or an integer of 1 or more, preferably an integer of 1 or more, more preferably of at least 3, even more preferably of at least 5; or
(3) the second asparaginyl ligase is butelase-1 or a variant thereof and said binding and ligation site for butelase-1 or variant thereof is located at the C-terminus of the third (poly)peptide and the N-terminus of the first (poly)peptide has the amino acid sequence X¹X²(X)_{q} with X 1 being G or H and X² being L, V or I, X being any amino acid, and q being 0 or an integer of 1 or more, preferably an integer of 1 or more, more preferably of at least 3, even more preferably of at least 5; or
(4) the second asparaginyl ligase is butelase-1 or a variant thereof and said binding and ligation site for butelase-1 or variant thereof is located at the C-terminus of the first (poly)peptide and the N-terminus of the third (poly)peptide has the amino acid sequence X¹X²(X)_{q} with X¹ being G or H and X² being L, V or I, X being any amino acid, and q being 0 or an integer of 1 or more, preferably an integer of 1 or more, more preferably of at least 3, even more preferably of at least 5.

It is understood that when in various embodiments VyPAL2 is the first asparaginyl ligase, butelase-1 is the second asparaginyl ligase and vice versa. This also applies if variants of VyPAL2 and/or butlease-1 are used.

In various other embodiments, the first and the second asparaginyl ligase are identical. In such embodiments, the different specificities necessary for selective ligation are provided by a change in reaction conditions. In various such embodiments, steps (i) and (ii) of the inventive methods are carried out at a first and a second pH-value that are different from each other, wherein the asparaginyl ligase has pH-dependent activity and specificity. However, in some alternative embodiments the first and second asparaginyl ligases may be different and steps (i) and (ii) of the inventive methods are still carried out at a first and a second pH-value that are different from each other.

In such embodiments where a pH change is used,
(1) the binding and ligation site for an asparaginyl ligase at the C-terminus of the first (poly)peptide is preferably bound and ligated by the asparaginyl ligase at the first pH value and the binding and ligation site for an asparaginyl ligase at the C-terminus of either the second or third (poly)peptide is preferably bound and ligated by the asparaginyl ligase at the second pH value; or
(2) the binding and ligation site for an asparaginyl ligase at the C-terminus of the first (poly)peptide is preferably bound and ligated by the asparaginyl ligase at the second pH value and the binding and ligation site for an asparaginyl ligase at the C-terminus of either the second or third (poly)peptide is preferably bound and ligated by the asparaginyl ligase at the first pH value.

In such methods, the first pH value may be a pH of about 6.0 or lower, preferably a pH in the range of 4.5-6.0, and the second pH value may be a pH of about 6.5 or higher, preferably a pH in the range of 6.5-7.4. Alternatively, the first and second pH Values may be exchanged such that the second pH value is a pH of about 6.0 or lower, preferably a pH in the range of 4.5-6.0, and the first pH value is a pH of about 6.5 or higher, preferably a pH in the range of 6.5-7.4.

In various embodiments where such pH change is employed, the asparaginyl ligase is VyPAL2 comprising or consisting of the amino acid sequence set forth in SEQ ID NO:1 or a variant thereof that has an amino acid sequence that has at least 80% sequence identity to the amino acid sequence set forth in SEQ ID NO:1 over its entire length.

In one further aspect, the invention relates to a method for (poly)peptide tandem ligation, the method comprising the steps of:
(i) contacting a first (poly)peptide (A) having at its C-terminus a binding and ligation site for an asparaginyl ligase with a second (poly)peptide (B) to be ligated to said first (poly)peptide and a first asparaginyl ligase (C) under conditions that allow ligation of the second (poly)peptide to the C- or N-terminus of the first (poly)peptide to yield a modified first (poly)peptide;
(ii) contacting the modified first (poly)peptide obtained in step (i) with a third (poly)peptide (D) to be ligated to said modified first (poly)peptide and a second asparaginyl ligase (E) under conditions that allow ligation of the third (poly)peptide to the C- or N-terminus of the first (poly)peptide to yield a dually modified first (poly)peptide;
steps (i) and (ii) are carried out at a first and a second pH-value that are different from each other, wherein the first pH value is a pH of about 6.0 or lower, preferably a pH in the range of 4.5-6.0, and the second pH value is a pH of about 6.5 or higher, preferably a pH in the range of 6.5-7.4, wherein the first and second asparaginyl ligases are different and wherein the asparaginyl ligase used at a pH of about 6 or lower is OaAEP1b comprising or consisting of the amino acid sequence set forth in SEQ ID NO:44 or a variant thereof that has an amino acid sequence that has at least 80% sequence identity to the amino acid sequence set forth in SEQ ID NO:44 over its entire length and the asparaginyl ligase used at a pH of about 6.5 or higher is (i) VyPAL2 comprising or consisting of the amino acid sequence set forth in SEQ ID NO:1 or a variant thereof that has an amino acid sequence that has at least 80% sequence identity to the amino acid sequence set forth in SEQ ID NO:1 over its entire length or (ii) butelase-1 comprising or consisting of the amino acid sequence set forth in SEQ ID NO:2 and variants thereof that share at least 80% sequence identity with the amino acid sequence set forth in SEQ ID NO:2 over their entire length.

In such methods, all the above-described embodiments of the more general methods are similarly applicable.

In various embodiments of the methods that include a pH change,
(1) the binding and ligation site for an asparaginyl ligase at the C-terminus of the first (poly)peptide has the amino acid sequence (X)ₒDX³X⁴(X)ₚ, wherein X is any amino acid, o is an integer of at least 2, X³ is an amino acid selected from A, C, F, G, H, K, N, Q, R, S, Y, preferably G, S, N, Q and R, more preferably G or S, and X⁴ is a hydrophobic or aromatic amino acid, preferably selected from L, I, V, F, C, W, Y and M, preferably L, I, and F, more preferably L or F, and p is 0 or an integer of 1 or more; and the binding an ligation site for an asparaginyl ligase at the C-terminus of the second or third (poly)peptide has the amino acid sequence (X)ₒNX³X⁴(X)ₚ, wherein X is any amino acid and o is an integer of at least 2, X 3 is G or S, and X 4 is a hydrophobic or aromatic amino acid, preferably selected from L, I, V, F, C, W, Y and M, preferably L or F, and p is 0 or an integer of 1 or more; or
(2) the binding and ligation site for an asparaginyl ligase at the C-terminus of the second or third (poly)peptide has the amino acid sequence (X)ₒDX³X⁴(X)ₚ, wherein X is any amino acid, o is an integer of at least 2, X 3 is an amino acid selected from A, C, F, G, H, K, N, Q, R, S, Y, preferably G, S, N, Q and R, more preferably G or S, and X⁴ is a hydrophobic or aromatic amino acid, preferably selected from L, I, V, F, C, W, Y and M, preferably L, I, and F, more preferably L or F, and p is 0 or an integer of 1 or more; and the binding an ligation site for an asparaginyl ligase at the C-terminus of the first (poly)peptide has the amino acid sequence (X)ₒNX³X⁴(X)ₚ, wherein X is any amino acid and o is an integer of at least 2, X³ is G or S, and X⁴ is a hydrophobic or aromatic amino acid, preferably selected from L, I, V, F, C, W, Y and M, preferably L or F, and p is 0 or an integer of 1 or more.

In such embodiments, the binding an ligation site having the amino acid sequence (X)ₒDX³X⁴(X)ₚ is preferably bound to by the asparaginyl ligase at a pH of about 6.0 or lower, preferably a pH in the range of 4.5-6.0, and the binding an ligation site having the amino acid sequence (X)ₒNX³X⁴(X)ₚ is preferably bound to by the asparaginyl ligase at a pH of about 6.5 or higher, preferably a pH in the range of 6.5 to 7.4.

In another aspect, the invention relates to a method for (poly)peptide cyclization, the method comprising the steps of:
(i) contacting (A) a first (poly)peptide having (i) at its C-terminus a binding and ligation site for an asparaginyl ligase with (B) a second (poly)peptide to be ligated to said first (poly)peptide having at its C-terminus a binding an ligation site for an asparaginyl ligase and (C) a first asparaginyl ligase under conditions that allow ligation of the second (poly)peptide to the C- or N-terminus of the first (poly)peptide to yield a modified first (poly)peptide;
(ii) contacting the modified first (poly)peptide obtained in step (i) with (E) a second asparaginyl ligase under conditions that allow ligation of the C-terminus of the modified first (poly)peptide to its N-terminus to yield a cyclized first (poly)peptide;
   wherein the first or second asparaginyl ligase is selected from VyPAL2 comprising or consisting of the amino acid sequence set forth in SEQ ID NO:1 and variants thereof that share at least 80% sequence identity with the amino acid sequence set forth in SEQ ID NO:1 over their entire length, and the other asparaginyl ligase is selected from butelase-1 comprising or consisting of the amino acid sequence set forth in SEQ ID NO:2 and variants thereof that share at least 80% sequence identity with the amino acid sequence set forth in SEQ ID NO:2 over their entire length.

In any of the methods described herein, at least one of the (poly)peptides to be ligated is further conjugated to an organic moiety. The organic moiety may be a pharmaceutically active agent or a detectable marker, such as a fluorescent marker or biotin.

The asparaginyl ligase consisting of SEQ ID NO:1 is also referred to herein as “VyPAL2” or “VyPAL2 active form/domain”. The asparaginyl ligase consisting of SEQ ID NO:2 is also referred to herein as “butelase-1” or “butelase-1 active form/domain”. The full-length polypeptide sequence of VyPAL2 is set forth in SEQ ID NO:3. The full-length polypeptide sequence of butelase-1 is set forth in SEQ ID NO:4.

### BRIEF DESCRIPTION OF THE DRAWINGS

FIG. 1. Bi-directional dual protein labeling by bio-orthogonal tandem ligation in N-to-C (a) or C-to-N (b) direction using butelase-1 and VyPAL2.

FIG. 2. Preparation of a cyclic affibody-drug conjugate by PAL-mediated tandem ligation-cyclization and drug conjugation.

FIG. 3. Kinetic studies on VyPAL2- and butelase 1-catalyzed intermolecular ligations. A) Peptide 1 (SEQ ID NO:7) containing the C-terminal NHV sequence was used at varying concentrations (50 — 800 μM) to react with peptide 2 (SEQ ID NO:8) at constant concentration (1 mM); B) Peptide 4 (SEQ ID NO:9) at a constant concentration (1.5 mM) was reacted with the GF-peptide 5 (SEQ ID NO:10) at varying concentrations (50-800 μM). C) Peptide 2 at a constant concentration (1.5 mM) was reacted with the -NGF peptide 7 (SEQ ID NO:11) at varying concentrations (50-800 μM). The ligation product 3 or 6 was confirmed by ESI-MS. The reaction rates were calculated from the consumption of the limiting substrate 1, 5 or 7. Initial rate (V₀) at different concentrations of the limiting substrate were used for Michaelis-Menten curve plotting. For each kinetic calculation, a lineweaver-Burk plot was also used to confirm the analysis.

FIG. 4. Bio-orthogonal protein dual labeling using VyPAL2 and butelase-1. A) N-to-C tandem ligation scheme. Fluorescein-peptide 9 (SEQ ID NO:12) was first ligated to the N terminus of Z_{EGFR} 8 (SEQ ID NO:35) via VML to give 10 which was then ligated with peptide 11 (SEQ ID NO:13) at C terminus via BML to give 12; B) HPLC analysis of N-to-C ligation. The ligation products 8, 10, 12 were purified by reverse-phase HPLC and analyzed via ESI-MS; C) C-to-N tandem ligation scheme. Mitochondrion-lytic peptide 11 (SEQ ID NO:13) is conjugated at C terminus of Z_{EGFR} 8 to give 13 via BML and then the fluorescein-peptide 9 (SEQ ID NO:12) is ligated to the N terminus of 13 to produce 12; D) HPLC analysis of N-to-C ligation. The ligation products 8, 13, 12 were purified by HPLC and analyzed by ESI-MS (8: calcd 8896.8, obsvd 8897.2; 10: calcd 9652.6, obsvd 9656.6; 12: calcd 10774.3, obsvd 10775.3 or 10775.9; 13: calcd 10017.8, obsvd 10018.47).

FIG. 5. Imaging and binding study of 12 on EGFR-overexpressing A431 cells. A) Schematic structure of the ubiquitin tagged with fluorescein (SEQ ID NO:36) and dual labeled affibody 12 (Fluorescein-SEQ ID NO:37-SEQ ID NO:13) with fluorescein on the N-terminus and mitochondrion-lytic peptide at the C-terminal end; B) Fluorescence microscopy analysis of 12 binding on A431 cells; C) Determination of K_{D} of 12 in binding to A431 cells using flowcytometry analysis.

FIG. 6. Cytotoxicity study of 12. A) Schematic structure of the KLA D-peptide 11 (SEQ ID NO:13) and dual labeled affibody 12 (Fluorescein-SEQ ID NO:37-SEQ ID NO:13) with fluorescein on the N-terminus and mitochondrion-lytic peptide at the C-terminal end; B) Microscopy analysis of 11 and 12 in MCF-7 and A431. Cells were treated either with phosphate buffer (as negative control) 11 or 12 for 72 h and then subjected to microscopy analysis after washing 3 times with PBS; C) IC₅₀ of MCF-7 and A 431 cells. Cells were both treated with 12 for 84 h and then MTT based viability test was performed to acquire the optical absorbance value to allow calculating the corresponding IC₅₀.

FIG. 7. Synthesis of a cyclic affibody-drug conjugate by using PAL-catalyzed orthogonal ligation and cyclization and oxime conjugation. A) i) Peptide 15 (SEQ ID NO:14) was tagged to the C-terminus of Z_{EGFR} 16 (Thz-SEQ ID NO:38) via VML to give 17 (90%). ii) The N-terminal cysteine of Z_{EGFR} 17 was deprotected using silver nitrate to afford 18 (95%). iii) Z_{EGFR} 18 was cyclized via BML to give 19 (70%). iv) Dox was attached to Z_{EGFR} 19 via oxime conjugation to give the final product 20 (80%). B) HPLC profiles of purified products. ESI-MS characterization data—16: calcd 8785.6, obsvd 8783.5; 17: calcd 9708.9, obsvd 9709.7; 18: calcd 9655.6, obsvd 9656.2; 19: calcd 9401.7, obsvd 9402.2; 20: calcd 9927.7, obsvd 9928.

FIG. 8. Cell imaging and cytotoxicity study of the cyclic affibody-dox conjugate 20. A) Fluorescent microscopy analysis of MCF-7 cells after treatment with 20, DOX and blank at room temperature for 30 min. B) Fluorescent microscopy analysis of A431 cells after treatment with 20, DOX and blank at room temperature for 30 min. For cell staining experiments in A) and B), nucleus was stained with 700 nM of DAPI; 10 μM DOX and 2 μM 20 were used respectively. Scal bar=50 μm. C) Cytotoxicity assay of the cPDC 20. Microscopy analysis of cells treated with 20. MCF-7 and A431 cells were treated with 0.2 μM of different molecules: DOX, unconjugated affibody 16, and 20 for 96 h. Scal bar=100 μm. D) Cytotoxic IC₅₀ of different compounds against MCF-7 and A431 cells. 20 exhibited a ˜10-fold enhanced toxicity on the EGFR-overexpressing A431 cell line as compared to doxorubicin.

FIG. 9. Screening of P1′-Asp peptides for cyclization by A) VyPAL2, B) butelase-1 and C) OaAEP1b. Quantitative summary of reaction yields of the reaction mixtures. For each reaction, 25 nM of the enzyme and 5 μM of the peptide substrate (21a-21i) were mixed and reacted at pH 4.5, 25° C. for 60 min. The positive control peptide 21j was performed at the same conditions except at pH 7.4. Average yields and SDs were calculated from experiments performed in triplicate. The amino acid sequence of the peptides used are set forth in SEQ ID Nos. 15-24.

FIG. 10. Ligation activity of three different ligases under different pH and substrate. Cyclization activity of A) VyPAL2, B) Butelase-1 or C) OaAEP1b towards substrate 21e-DSL and 21j-NGL; Fold difference was calculated using the cyclization rate of 21j-NGL divided by that of 21e-DSL. All the results were the average reaction rate at different pH in triplicate experiments based on MALDI-TOF analysis. Light grey text in the “Fold difference” axis marks to the maximum fold difference.

FIG. 11. Enzymatic activity of three different ligases on the cyclization reaction of P1-Asp peptide 21e-DSL or 21a-DGL and P1-Asn-peptide 21j-NGL. A) Scheme of PAL-mediated P1-Asp peptide cyclization; B) Enzymatic kinetics of VyPAL2 in cyclizing 21e-DSL at pH 4.5; C) Enzymatic kinetics of VyPAL2 in cyclizing 21e-DSL at pH 7.0; D) Enzymatic kinetics of OaAEP1b in cyclizing 21a-DGL at pH 5.5; E) Enzymatic kinetics of Butelase-1 in cyclizing 21e-DSL at pH 4.5. F) Scheme of PAL-mediated cyclization of peptide 21j-NGL; G). Enzymatic kinetics of butelase-1 in cyclizing 21j-NSL at pH 4.5; H)

Enzymatic kinetics of VyPAL2 in cyclizing 21j-NSL at pH 4.5; I) Enzymatic kinetics of butelase-1 in cyclizing 21j-NSL at pH 7.4; J) Enzymatic kinetics of OaAEP1b in cyclizing 21j-NSL at pH 7.0. Sequences of peptides used are set forth in SEQ ID Nos. 15, 19, 24.

FIG. 12. VyPAL-mediated macrocyclization of sfGFP at pH 4.5. A) Scheme of VyPAL2-mediated cyclization of sfGFP-DSL-His₆ 23 (SEQ ID NO:39); B) HPLC monitoring of sfGFP 23 cyclization at 0 h (upper) and 3 h (lower). ESI-MS characterization (23: Calcd: 27408.8, Obsvd 27400.2; 24: Calcd. 26359.7, Obsvd. 26352.3).

FIG. 13. Affibody 25 (SEQ ID NO:40) tagging with fluorescein-peptide 26 (GVCit-PABC-fluorescein where Cit-PABC=citrulline-p-aminobenzylcarbamate) via VyPAL2-mediated ligation. Affibody 25 containing the C-ter DSL tag was reacted with the fluorescein-peptide 26 to produce ligation product 27 using VyPAL2. A) Reaction scheme; B) HPLC analysis of the ligation reaction. ESI-MS data of affibody 25: Calcd. 9045.1, Obsvd. 9046.7; The product 27: Calcd. 9707.1, Obsvd. 9709.3.

FIG. 14. Affibody 25 tagging with fluorescein-peptide 28 (SEQ ID NO:25) via VyPAL2-mediated ligation. Affibody 25 containing the C-ter DSL tag was reacted with the fluorescein-peptide 28 using VyPAL2. ESI-MS data of product 29: Calcd. 9883.1, Obsvd. 9883.0.

FIG. 15. Affibody 31 (SEQ ID NO:41) tagging with Dox-peptide 30 via VyPAL2-mediated ligation. A) Reaction scheme; B) HPLC analysis of the ligation reaction. ESI-MS data of affibody 31: Calcd. 9014.1, Obsvd. 9014.6; The product 32: Calcd. 9833.1, Obsvd. 9833.4.

FIG. 16. Dual labelling of sfGFP (SEQ ID NO:42) by N-to-C tandem ligation using VyPAL2. A) Design of the terminal truncated sfGFP for tandem ligation. Upper panel: Schematic illustration of sfGFP 33 truncation sites at the C terminus; Lower panel: comparison of the N and C termini distance between wildtype sfGFP and truncated sfGFP 33; B) sfGFP N-terminal ligation with cancer targeting peptides 34a-34f. Reaction yields were estimated using HPLC by comparing the areas of peaks corresponding to the starting material and product which were characterized by ESI-MS; C) sfGFP C-terminal ligation with Dox-peptides 30. Peptide sequences of 34a-34f are set forth in SEQ ID Nos. 26-31.

FIG. 17. VyPAL-mediated one-pot tandem ligation on sfGFP. The reaction was profiled using HPLC and product 38 was characterized using ESI-MS (Calcd. 27641.2, Obsvd. 27637.4). Peptides 28 and 37 have the aa sequences set forth in SEQ ID Nos. 25 and 32.

FIG. 18. Protein dual labelling using VyPAL2. A) C-to-N tandem ligation scheme. peptide 41 (SEQ ID NO:33) was first ligated to the C terminus of Z_{EGFR} 40 (Thz-SEQ ID NO:43) via VyPAL2-mediated ligation at pH 4.5 to give 42 which was then ligated with peptide 44 (SEQ ID NO:34) at N terminus via VyPAL-mediated ligation at pH 7.5 to give 45. The products 40, 42, 43, 45 were purified by HPLC and analyzed via ESI-MS. (40: Calcd 8928.5, Obsvd 8927.1; 42: Calcd 9212.5, Obsvd 9211.9; 43: Calcd 9155.9 Obsvd 9156.8; 45: Calcd 9631.9, Obsvd 9632.8).

FIG. 19. Affibody dual labelling using VyPAL2. A) C-to-N tandem ligation scheme. Peptide 30 was first ligated to the C terminus of Z_{EGFR} 40 via VyPAL2-mediated ligation at pH 4.5 to give 46 which was then ligated with peptide 9 at the N terminus via VyPAL-mediated ligation at pH 7.4 to give 48; B) HPLC analysis of ligation reactions. The products 46, 47 and 48 were purified by HPLC and analyzed by ESI-MS. (39: calcd 8873.8, obsvd 8873.2; 40: calcd 8928.5, obsvd 8927.1; 46: calcd 9715.9, obsvd 9718.0; 47: calcd 9659.9, obsvd 9659.0; 48: calcd 10415.7 obsvd 10417.8).

FIG. 20. Affibody dual labelling using C-to-N tandem ligation by OaAEP1 b and butelase-1. Peptide 28 (SEQ ID NO:25) was first ligated to the C terminus of Z_{EGFR} 39 via OaAEP-mediated ligation at pH 6.0 to give 49 which was then ligated with peptide 9 (SEQ ID NO:12) at the N terminus via butelase-mediated ligation or VyPAL-mediated ligation at pH 7.5 to give 50. The ligation products 49, 50 were purified by HPLC and analysed by ESI-MS. (49: Calcd. 9767.5, Obsvd. 9765.3; 50: Calcd. 10467.5, Obsvd. 10466.1).

FIG. 21. Confocal analysis of MCF-7 and A431 cells treated with affibody 48. Fluorescent microscopy analysis of MCF-7 and A431 cells after treatment with blank, Dox, 47 and 48 for 30 min. For cell staining experiments, cells were fixed with 4% formaldehyde and permeabilized with 0.2% tritonX-100. The cell nucleus was stained with 1 μM of DAPI. Dox and protein 47, 48 diluted in PBS were all added in 10 μM. All the staining experiments were performed for 30 min at room temperature; Scale bar=50 μm.

FIG. 22. Cytotoxicity assay of the affibody-dox conjugate 48. A) Microscopy analysis of cells treated with the affibody-drug conjugate. MCF-7 and A431 cells were treated with 0, 0.3 and 0.4 μM of protein 48 for 36 h; B) Cytotoxicity IC₅₀ of the different compounds against MCF-7 and A431 cells. About 10-fold enhanced toxicity was observed on the EGFR-overexpressing A431 cell line; C) Summarization of IC₅₀ of Dox and protein 48 towards MCF-7 and A431 cells.

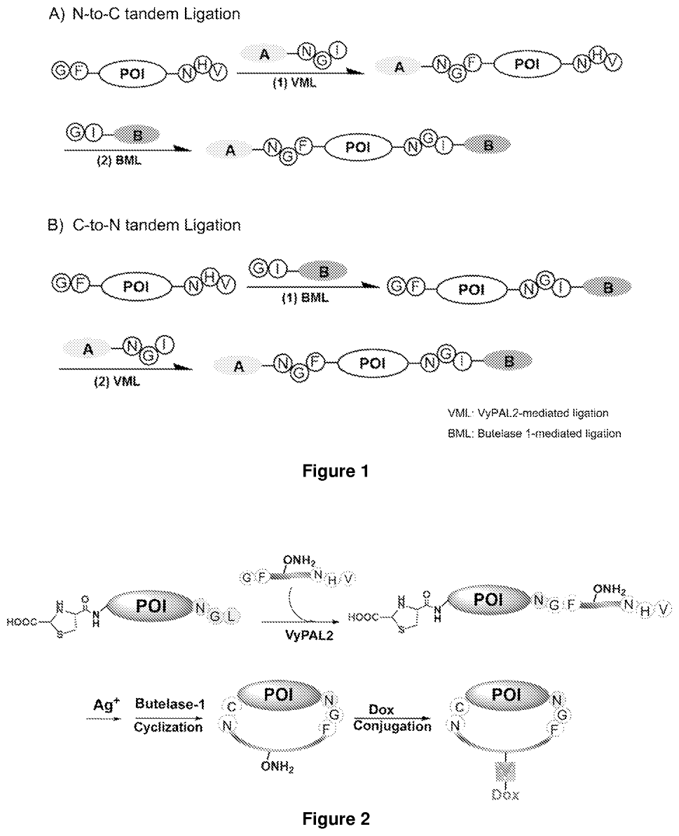

### DETAILED DESCRIPTION

The present invention is based on the inventors' finding that previously identified asparaginyl ligases butelase-1 and VyPal2 can be advantageously used for (poly)peptide tandem ligation and thus allow the synthesis of dually modified peptides and polypeptides that have multiple applications in therapeutics and diagnostics.

The present invention is directed to methods for (poly)peptide tandem ligation. These methods comprise the steps of:
contacting a first (poly)peptide (A) having at its C-terminus a binding and ligation site for an asparaginyl ligase with a second (poly)peptide (B) to be ligated to said first (poly)peptide and a first asparaginyl ligase (C) under conditions that allow ligation of the second (poly)peptide to the C- or N-terminus of the first (poly)peptide to yield a modified first (poly)peptide; and
contacting the modified first (poly)peptide obtained in step (i) with a third (poly)peptide (D) to be ligated to said modified first (poly)peptide and a second asparaginyl ligase (E) under conditions that allow ligation of the third (poly)peptide to the C- or N-terminus of the first (poly)peptide to yield a dually modified first (poly)peptide.

The asparaginyl ligases according to the present invention exhibit protein ligation activity, i.e. are capable of forming a peptide bond between two amino acid residues, with these two amino acid residues being located on the same or different peptides or proteins. Accordingly, in various embodiments, the asparaginyl ligase may have cyclase activity. In various embodiments, this protein ligation or cyclase activity includes an endopeptidase activity, i.e. the polypeptide form a peptide bond between two amino acid residues following cleavage of an existing peptide bond. This means that cyclization need not to occur between the termini of a given peptide but can also occur between internal amino acid residues, with the amino acids C-terminal or N-terminal to the amino acid used for cyclization being cleaved off. The asparaginyl ligases disclosed herein are “Asx-specific” in that the amino acid C-terminal to which ligation occurs, i.e. the C-terminal end of the peptide that is ligated, is either asparagine (Asn or N) or aspartic acid (Asp or D).

The asparaginyl ligases may be naturally occurring enzymes and may be provided in isolated form. “Isolated”, as used herein, relates to the polypeptide in a form where it has been at least partially separated from other cellular components it may naturally occur or associate with. The asparaginyl ligases may be recombinant polypeptides, i.e. polypeptides produced in a genetically engineered organism that does not naturally produce said polypeptide. Both native and recombinant polypeptides may be post-translationally modified by N-linked glycosylation.

The first and second asparaginyl ligase used in these methods are selected from VyPAL2 comprising or consisting of the amino acid sequence set forth in SEQ ID NO:1 and variants thereof that share at least 80% sequence identity with the amino acid sequence set forth in SEQ ID NO:1 over their entire length, and butelase-1 comprising or consisting of the amino acid sequence set forth in SEQ ID NO:2 and variants thereof that share at least 80% sequence identity with the amino acid sequence set forth in SEQ ID NO:2 over their entire length. If the asparaginyl ligase comprises SEQ ID NO:1, it can be the native VyPAL2 sequence as set forth in SEQ ID NO:3 or any fragment thereof that comprises SEQ ID NO:1. If the asparaginyl ligase comprises SEQ ID NO:2, it can be the native butelase-1 sequence as set forth in SEQ ID NO:4 or any fragment thereof that comprises SEQ ID NO:2.

The variants are at least 80%, preferably at least 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.25%, or at least 99.5% identical to the amino acid sequence set forth in SEQ ID NO:1 or 2 over their entire length. The variants may also be fragments of the respective reference sequence of SEQ ID NO:1 or 2 that retain their activity. Such fragments are typically C- and/or N-terminally truncated versions of the reference sequence and preferably comprise the determinants for the activity of the enzyme as defined herein below. The same definition of variants applies to the respective full-length sequences set forth in SEQ ID Nos. 3 and 4.

In various embodiments, the variant may be a precursor of the mature enzyme.

The identity of nucleic acid sequences or amino acid sequences is generally determined by means of a sequence comparison. This sequence comparison is based on the BLAST algorithm that is established in the existing art and commonly used (cf. for example Altschul et al. (1990) “Basic local alignment search tool”, J. Mol. Biol. 215:403-410, and Altschul et al. (1997): “Gapped BLAST and PSI-BLAST: a new generation of protein database search programs”; Nucleic Acids Res., 25, p. 3389-3402) and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art.

A comparison of this kind also allows a statement as to the similarity to one another of the sequences that are being compared. This is usually indicated as a percentage identity, i.e. the proportion of identical amino acid residues at the same positions or at positions corresponding to one another in an alignment. Indications of identity can be encountered over entire polypeptides or only over individual regions. Identical regions of various amino acid sequences are therefore defined by way of matches in the sequences. Such regions often exhibit identical functions. They can be small, and can encompass only a few amino acids. Small regions of this kind often perform functions that are essential to the overall activity of the protein. It may therefore be useful to refer sequence matches only to individual, and optionally small, regions. Unless otherwise indicated, however, indications of identity herein refer to the full length of the respectively indicated nucleic acid sequence or amino acid sequence.

In various embodiments, the variants of butelase-1 and VyPAL2 described herein comprise the amino acid residue N at the position corresponding to position 19 of SEQ ID NO:1; and/or the amino acid residue H at the position corresponding to position 124 of SEQ ID NO:1; and/or the amino acid residue C at the position corresponding to position 166 of SEQ ID NO:1. In various embodiments, at least the catalytic dyad formed by the amino acid residue H at the position corresponding to position 124 of SEQ ID NO:1 and the amino acid residue C at the position corresponding to position 166 of SEQ ID NO:1 is present, preferably in combination with the amino acid residue N at the position corresponding to position 19 of SEQ ID NO:1, thus forming the complete catalytic triad. It has been found that these amino acid residues are necessary for the catalytic activity (ligase activity) of the polypeptide. In preferred embodiments, the variants thus comprise at least two, more preferably all three of the above indicated residues at the given or corresponding positions.

All amino acid residues are generally referred to herein by reference to their one letter code and, in some instances, their three-letter code. This nomenclature is well known to those skilled in the art and used herein as understood in the field.

In various embodiments, the variants referred to herein comprise the amino acid residue A at the position corresponding to position 126. In various embodiments, the variants referred to herein comprise the amino acid residue A or P, preferably P, at the position corresponding to position 127 of SEQ ID NO:1. Alternatively, the amino acid residue at the position corresponding to position 126 of SEQ ID NO:1 may be G. In these embodiments, the amino acid residue at the position corresponding to position 127 of SEQ ID NO:1 is preferably A. These motifs AP, AA and GA are also referred to herein as Ligase Activity Determinant 2 (LAD2), as they are critical determinants for the ligase activity. In various embodiments the motif at the positions corresponding to positions 126 and 127 of SEQ ID NO:1 is not GP, but either AP, AA or GA.

In various embodiments, the variants referred to herein comprise the amino acid residue W or Y at the position corresponding to position 195, the amino acid residue I or V at the position corresponding to position 196, and the amino acid residue T, A or V at the position corresponding to position 197 of SEQ ID NO:1. It has been found that this motif W-I/V--T/A/V, also referred to herein as Ligase Activity Determinant 1 (LAD1), is also a critical determinant for the ligase activity. In addition to the known gatekeeper position that corresponds to position 196 in SEQ ID NO:1, it has been found that also positions 195 and 197, in particular 195, are relevant for determining ligase/endopeptidase activity.

In various embodiments, the variants referred to herein comprise the amino acid residues R at the position corresponding to position 21, H at the position corresponding to position 22, D at the position corresponding to position 123, E at the position corresponding to position 164, S at the position corresponding to position 194, and D at the position corresponding to position 215 of SEQ ID NO:1. These amino acid residues are also referred to herein as “51 pocket”, which has also been found to be involved in ligase activity.

In various embodiments, the variants referred to herein comprise the amino acid residues C at the positions corresponding to positions 199 and 212 of SEQ ID NO:1. These two residues typically form a disulfide bridge in the mature polypeptide, which contributes to ligase activity.

The variants of the invention may, in various embodiments, comprise further more or less invariable sequence elements, such as the poly-Pro loop (PPL). Said loop has the consensus sequence P/A-G/T/S-X-P/E-G/D/P-V/F/A/P-P-L/P/A/E-E and comprises at least 2 and up to 5 proline residues. Typical are 2, 3, 4 or 5 proline residues at the indicated positions. The PPL occupies positions 200-208 of SEQ ID NO:1.

Another motif that may be present in the variants of the invention is the so-called MLA motif spanning residues 244-249 of SEQ ID NO:1. This may have the sequence KKIAYA or NKIAYA (SEQ ID Nos. 5 and 6).

In various embodiments, the variants of the invention comprise the LAD1 and LAD2 motifs as described above. In further embodiments, they additionally comprise one, two, three or all four of the 51 pocket, SS bridge, PPL and MLA motif, as defined above. The presence of these motifs ensures their functionality as ligases even if other parts of the sequence are modified.

In various embodiments, the variants comprise fragments of the asparaginyl ligases described herein, with said fragments retaining enzymatic activity. It is preferred that they have at least 50%, more preferably at least 70, most preferably at least 90% of the protein ligase and/or cyclase activity of the initial molecule, preferably of the polypeptide having the amino acid sequence of SEQ ID NO:1 or 2. The fragments are preferably at least 150 amino acids in length, more preferably at least 200 or 250. It is further preferred that these fragments comprise the amino acids N, H and C at positions corresponding to positions 19, 124 and 166 of SEQ ID NO:1 as well as the above-defined LAD1, LAD2 and optionally also any one or more of the S1 pocket, the PPL, MLA motif and disulfide bridge contained in the initial molecule. Preferred fragments therefore comprise amino acids 19-197, more preferably 19-212, most preferably 19-249 corresponding to the respective positions in the amino acid sequence set forth in SEQ ID NO:1.

The variants of VyPAL2 described herein are preferably designed such that the specificity and selectivity of VyPAL2 are retained and the same applies to variants of butelase-1. It is furthermore preferred that such variants do not contain modifications that render VyPAL2 more similar to butelase-1 and vice versa. In preferred embodiments, such variants have therefore the same or lower degree of sequence identity to the respective other asparaginyl ligase than the starting enzyme.

It is preferred that the variants of the invention have at least 50%, more preferably at least 70, most preferably at least 90% of the protein ligase activity of the enzyme they are derived from.

In various embodiments, the variants are capable of ligating/cyclizing a given peptide with an efficiency of 60% or more, preferably 80% or more, preferably at a pH of 5.5 or higher. The cyclization activity may also be determined at pH values of 6.0, 6.5, 7.0, 7.5 or higher. This is relevant, since at low pH conditions, such as below pH 5, the ligases may exhibit a certain degree of endopeptidase activity.

The variants of butelase-1 and VyPAL2 according to the embodiments described herein can comprise amino acid modifications, in particular amino acid substitutions, insertions, or deletions. Such variants are, for example, further developed by targeted genetic modification, i.e. by way of mutagenesis methods, and optimized for specific purposes or with regard to special properties (for example, with regard to their catalytic activity, stability, etc.). If such additional modifications are introduced into the asparaginyl ligases of the invention, these preferably do not affect, alter or reverse the sequence motifs detailed above, i.e. the catalytic residues, the LAD1 and LAD2 motifs. This means that the above-defined features of these residues/motifs are not changed by these additional mutations beyond that what is defined above. It can be further preferred that additionally one, two, three or all four of the S1 pocket, SS bridge, PPL and MLA motif are retained without additional modifications, i.e. modifications going beyond those detailed above.

In various embodiments, the polypeptides having ligase/cyclase activity may be post-translationally modified, for example glycosylated. Such modification may be carried out by recombinant means, i.e. directly in the host cell upon production, or may be achieved chemically or enzymatically after synthesis of the polypeptide, for example in vitro.

For example, butelase-1 (SEQ ID NO:4) is glycosylated at N94 and N286 with bulky heterogeneous glycans, which results in an increase of additional mass of about 6 kDa. The recombinant VyPAL2 (SEQ ID NO:3) is glycosylated at positions N102, N145 and N237, with small glycans, and which results in an additional increased mass of about 3 kDa. The polypeptides of the invention may thus be glycosylated with bulky, heterogeneous glycans, for example at positions corresponding to positions N94 and N286 of SEQ ID NO:4 or with small glycans at positions corresponding to positions N102, N145 and N237 of SEQ ID NO:3.

The term “(poly)peptide”, as used herein, refers to peptides and polypeptides. “Polypeptide”, as used herein, relates to polymers made from amino acids connected by peptide bonds. The polypeptides, as defined herein, can comprise more than 50 amino acids, preferably 100 or more amino acids. “Peptides”, as used herein, relates to polymers made from amino acids connected by peptide bonds. The peptides, as defined herein, can comprise 2 or more amino acids, preferably 5 or more amino acids, more preferably 10 or more amino acids, for example 10 to 50 amino acids.

In various embodiments, the first (poly)peptide is a polypeptide or protein and comprises more than 100 amino acids. In various embodiments, the second and/or third (poly)peptide are peptides and comprise 5 to 50, preferably 5 to 30 amino acids. In various embodiments, the first (poly)peptide is the (poly)peptide to be modified and the second and third (poly)peptides are the modifications that are to be ligated to the N- and C-terminus, respectively, of the first (poly)peptide.

In order to avoid side reactions, the methods described herein are typically performed in two separate steps. In the first step, the second (poly)peptide is ligated to the first (poly)peptide by a first asparaginyl ligase. Non-ligated peptides may be removed after this step and the ligation product isolated. In a second step, the product of the first step is then ligated to the third (poly)peptide to yield a linear dually modified (poly)peptide, typically with the second and third (poly)peptide ligated to the N- and C-terminus, respectively, of the first (poly)peptide or vice versa, or it is cyclized. In any case, for this second step a second asparaginyl ligase is used. The first and second asparaginyl ligases used have different substrate specificities to allow targeted ligation and prevent or reduce the production of undesired side products. This difference in specificity can be provided by use of different asparaginyl ligases or by changing the reaction conditions, such as pH, such that the specificity of the respective asparaginyl ligase is changed.

Even in case two different asparaginyl ligases are used, these differences in specificities are typically not absolute, but rather relate to preferential recognition and ligation of one motif over another, with said preference being, for example at least 1.5-fold, at least 2-fold, at least 3-fold, at least 5-fold or at least 10-fold. The higher said preference, the higher the specificity and the lower the production of side products. To achieve this specificity in the ligation reaction, (poly)peptides with different asparagine-containing motifs are used that are preferentially recognized and ligated by one of the two ligases employed. More detailed information on the respective motifs is provided herein below. If pH-dependent changes in ligase specificity are exploited in the methods, one of the to be ligated motifs is typically an aspartic acid-containing motif and the other is an asparagine-containing motif. More detailed information on such motifs will also be provided herein below.

In the first step of the methods described herein, the C-terminus of the second (poly)peptide may be ligated to the N-terminus of the first (poly)peptide. In such embodiments, the first asparaginyl ligase would have higher specificity for the motif at the C-terminus of the second (poly)peptide than the motif at the C-terminus of the first (poly)peptide. The main product of such a reaction would thus be a ligation product where the second (poly)peptide is linked to the N-terminus of the first (poly)peptide by a peptide bond. It is understood that depending on the motif present at the C-terminus of the first (poly)peptide and the N-terminal amino acids of the second (poly)peptide a side product that is the ligation product of the C-terminus of the first (poly)peptide to the N-terminus of the second (poly)peptide may be obtained. Further side products may be ligation products of the one of the two reaction partners to other molecules of the same type. However, it is an object of the methods of the invention to minimize the production of such side products by the selection of motifs that are with high preference recognized by the asparaginyl ligase employed relative to other motifs present and to further control the reaction by adjustment of reaction conditions, including, for example, the amounts/ratios of reaction partners used.

Alternatively, in said first step the main reaction may be the reaction of the C-terminus of the first (poly)peptide with the N-terminus of the second (poly)peptide.

In the second step of the methods described herein, either the C-terminus of the ligation product of the first step is ligated to the N-terminus of the third (poly)peptide or the C-terminus of the third (poly)peptide is ligated to the N-terminus of the first (poly)peptide. This may be dependent on whether the second (poly)peptide has been ligated to the N- or C-terminus of the first (poly)peptide in the first step, as the third (poly)peptide is preferably ligated to that end of the first (poly)peptide that has not been ligated to the second (poly)peptide to yield a dually modified, i.e. a C- and N-terminally modified, (poly)peptide.

Again, in such second step there may be side products, as even the ligation site of the first step may, due to the presence of the asparagine/aspartate residue in said site, be recognized and cleaved by the second asparaginyl ligase depending on its specificity and the actual motif generated by the first ligation. Also possible is the generation of ligation products of the same molecule.

However, as demonstrated in the examples, the different specificities of the asparaginyl ligases employed allow a clear preference for the ligation product of choice which can be further fine-tuned by controlling reaction conditions.

In various embodiments of the methods described herein, the second (poly)peptide has at its C-terminus a binding and ligation site for the first asparaginyl ligase and is ligated to the N-terminus of the first (poly)peptide by the first asparaginyl ligase. Such methods are also referred to herein as “N-to-C tandem ligation”, since the N-terminus of the first (poly)peptide is ligated first. In such embodiments, the binding and ligation site for an asparaginyl ligase at the C-terminus of the first (poly)peptide may be for the second asparaginyl ligase and the third (poly)peptide can then be ligated to the C-terminus of the first (poly)peptide by the second asparaginyl ligase.

In various other embodiments, the second (poly)peptide is ligated to the C-terminus of the first (poly)peptide by the first asparaginyl ligase, wherein the binding and ligation site for an asparaginyl ligase at the C-terminus of the first (poly)peptide is for the first asparaginyl ligase. Such methods are also referred to herein as “C-to-N tandem ligation”, since the C-terminus of the first (poly)peptide is ligated first. In such embodiments, the third (poly)peptide may have at its C-terminus a binding and ligation site for the second asparaginyl ligase and may be ligated to the N-terminus of the first (poly)peptide by the second asparaginyl ligase. Alternatively, in such embodiments, the second (poly)peptide may have at its C-terminus a binding and ligation site for the second asparaginyl ligase and may be ligated to the N-terminus of the third (poly)peptide by the second asparaginyl ligase. However, such latter embodiments where the third (poly)peptide is ligated to the already ligated second (poly)peptide are not preferred as they do not yield a dually modified first (poly)peptide (since this requires that both the C- and the N-terminus of the first (poly)peptide are ligated to another peptide).

In various embodiments of the methods described herein, the first and second asparaginyl ligases are different and the first asparaginyl ligase is VyPAL2 or a variant thereof and the second asparaginyl ligase is butelase-1 or a variant thereof; or vice versa. It has been found the VyPAL2 and butelase-1, although both are highly efficient asparaginyl ligases, differ sufficiently in their substrate specificity that they can be employed for bio-orthogonal and dual ligation.

The binding and ligation site for VyPAL2 or a variant thereof may have, in various embodiments, the amino acid sequence (X)ₒNX³X⁴(X)ₚ, wherein X is any amino acid and o is an integer of at least 2, X³ is an amino acid selected from A, C, F, G, H, K, N, Q, R, S, Y, preferably G, S, N, Q and R, more preferably G or S, and X 4 is a hydrophobic or aromatic amino acid, preferably selected from L, I, V, F, C, W, Y and M, preferably L, I, and F, more preferably L or F, and p is 0 or an integer of 1 or more. The preferred motif for VyPAL2 is (X)ₒNSL or (X)ₒNGF. (X)ₒNGF is a motif where the difference in specificity between VyPAL2 and butelase-1 is pronounced (about 3-fold higher catalytic activity of VyPAL2 relative to that of butelase-1) although said motif is still recognized and cleaved by butelase-1. The substrate specificity of VyPAL2 has also been described in Hemu et al. [42].

When reference is herein to “any amino acid”, it is typically meant that the respective amino acid can be any naturally occurring amino acid, preferably any one of the 20 proteinogenic amino acids G, A, V, L, I, M, C, F, W, Y, R, K, H, E, D, Q, N, P, S and T.

The binding and ligation site for butelase-1 or a variant thereof may have, in various embodiments, the amino acid sequence (X)ₒNX³X⁴(X)ₚ, wherein X is any amino acid and o is an integer of at least 2, X³ is H, and X⁴ is a hydrophobic or aromatic amino acid, preferably selected from L, I, V, F, C, W, Y and M, preferably V, and p is 0 or an integer of 1 or more. The preferred motif for butelase-1 is (X)ₒNHV. Said motif shows a high difference in specificity between butelase-1 and VyPAL2 and is about 18-fold more effectively bound and cleaved by butelase-1 than by VyPAL2.

Said amino acid sequences (X)ₒNX³X⁴(X)ₚ are preferably located at or near the C-terminus of the peptide to be ligated or cyclized, as all amino acids C-terminal to the N will be cleaved off during ligation/cyclization. Accordingly, in all afore-mentioned embodiments, p is preferably 0 or an integer of up to 20, preferably up to 5. Particularly preferred are embodiments, where p is 0 or 1, most preferably with p=0. It has also been found that X³ and X⁴ need to be present to allow efficient ligation/cyclization. It has however also been found that amidated versions of the sequence (X)ₒN/D*, with the asterisk indicating the amidation can also serve as substrates. Although not preferred the respective motifs may thus also be (X)ₒN or (X)ₒD.

The N-terminal part of the peptide to be ligated preferably comprises the amino acid sequence X¹X²(X)_{q}, wherein X can be any amino acid; X¹ can be any amino acid with the exception of Pro; X² can be any amino acid, but preferably is an amino acid selected from V, I, L, C, W, A, T, F, Y, M, and Q; and q is 0 or an integer of 1 or more, preferably an integer of 1 or more, more preferably of at least 3, even more preferably of at least 5.

Preferred are in the X¹ position are for both asparaginyl ligases in the following order: G=H>M=W=F=R=A=I=K=L=N=S=Q=C>T=V=Y>D=E. “=” indicates that the respective amino acids are similarly preferred, while “>” indicates a preference of the amino acids listed before the symbol over the ones listed after the symbol.

Preferred in the X² position are for butelase-1 in the following order : I>L>V>C>T>W>A=F>Y>M>Q>S. Less preferred in the X² position are P, D, E, G, K , R, N and H. Particularly preferred in the X¹ position are G and H and in the X² position L, V, I and C, such as the dipeptide sequences GL, GV, GI, GC, HL, HV, HI and HC. It has however been found that, for example HV and GV are less efficient than GI, therefore GI is a preferred N-terminal motif for the peptide nucleophile.

Preferred in the X² position is for VyPAL2 the residue F, since for such motifs the difference in specificity is maximized between butelase-1 and VyPAL2. For example, it was shown that VyPAL2 has an about 5-fold higher catalytic activity than butelase-1 towards a peptide with the N-terminal sequence GF.

In preferred embodiments, the peptide to be ligated or cyclized thus comprises in N- to C-terminal orientation, the amino acid sequence X¹X²(X)_{q}(X)ₒNX³X⁴(X)ₚ, wherein X, X¹, X², X³, X⁴, o, p, and q are defined as above, with o preferably being at least 7. In various embodiments, (1) q is 0 and o is an integer of at least 7; and/or (2) X¹ is G or H; and/or (3) X² is L, V, I, F or C depending on the desired specificity; and/or (4) p is 0 but not more than 20, preferably 0-7. In some embodiments, for butelase-1 X³X⁴(X)ₚ is HX⁴(X)ₚ or HV(X)ₚ, preferably HX⁴ or HV. In some embodiments, for VyPAL2 X³X⁴(X)ₚ is X³F(X)ₚ or GF(X)ₚ or SL(X)ₚ or GL(X)ₚ, preferably GF.

The preferred motif for VyPAL2-mediated ligation (VML) is NSL or NGF or NGL, and the preferred motif for butelase-1 mediated ligation (BML) is NHV. Said motifs are preferably located at the C-termini of the respective (poly)peptide. Any of the two may be located at the C-terminus of the first (poly)peptide and the respective other is then located at the C-terminus of the second or third (poly)peptide.

In various embodiments,
(1) the first asparaginyl ligase is VyPAL2 or a variant thereof and said binding and ligation site for VyPAL2 or variant thereof is located at the C-terminus of the second (poly)peptide and the N-terminus of the first (poly)peptide optionally has the amino acid sequence X¹F(X)_{q}; or
(2) the first asparaginyl ligase is VyPAL2 or a variant thereof and said binding and ligation site for VyPAL2 or variant thereof is located at the C-terminus of the first (poly)peptide and the N-terminus of the second (poly)peptide optionally has the amino acid sequence X¹F(X)_{q}; or
(3) the second asparaginyl ligase is VyPAL2 or a variant thereof and said binding and ligation site for VyPAL2 or variant thereof is located at the C-terminus of the third (poly)peptide and the N-terminus of the first (poly)peptide optionally has the amino acid sequence X¹F(X)_{q}; or
(4) the second asparaginyl ligase is VyPAL2 or a variant thereof and said binding and ligation site for VyPAL2 or variant thereof is located at the C-terminus of the first (poly)peptide and the N-terminus of the third (poly)peptide optionally has the amino acid sequence X¹F(X)_{q}.

In all the afore-described embodiments (1)-(4), the binding and ligation site for VyPAL2 or variant thereof may be as defined above, in particular (X)ₒNSL or (X)ₒNGF, preferably (X)ₒNGF.

In various embodiments,
(5) the first asparaginyl ligase is butelase-1 or a variant thereof and said binding and ligation site for butelase-1 or variant thereof is located at the C-terminus of the second (poly)peptide and the N-terminus of the first (poly)peptide has the amino acid sequence X¹X²(X)_{q} with X¹ preferably being G or H and X² preferably being L, V or I, more preferably GI(X)_{q}; or
(6) the first asparaginyl ligase is butelase-1 or a variant thereof and said binding and ligation site for butelase-1 or variant thereof is located at the C-terminus of the first (poly)peptide and the N-terminus of the second (poly)peptide has the amino acid sequence X¹X²(X)_{q} with X¹ preferably being G or H and X² preferably being L, V or I, more preferably GI(X)_{q}; or
(7) the second asparaginyl ligase is butelase-1 or a variant thereof and said binding and ligation site for butelase-1 or variant thereof is located at the C-terminus of the third (poly)peptide and the N-terminus of the first (poly)peptide has the amino acid sequence X¹X²(X)_{q} with X¹ preferably being G or H and X² preferably being L, V or I, more preferably GI(X)_{q}; or
(8) the second asparaginyl ligase is butelase-1 or a variant thereof and said binding and ligation site for butelase-1 or variant thereof is located at the C-terminus of the first (poly)peptide and the N-terminus of the third (poly)peptide has the amino acid sequence X¹X²(X)_{q} with X¹ preferably being G or H and X² preferably being L, V or I, more preferably GI(X)_{q}.

In all the afore-described embodiments (5)-(8), the binding and ligation site for butelase-1 or variant thereof may be as defined above, in particular (X)ₒNHV.

It is understood that when in various embodiments VyPAL2 is the first asparaginyl ligase, butelase is the second asparaginyl ligase and vice versa. This also applies if variants of VyPAL2 and/or butelase-1 are used. Accordingly, the following embodiments described above may be combined: embodiment (1) and embodiment (8); embodiment (2) and embodiment (7); embodiments (3) and (6); embodiments (4) and (5).

In various other embodiments, the first and the second asparaginyl ligase are identical and are preferably VyPAL2 or a variant thereof, as herein described. In such embodiments, the different specificities necessary for orthogonal ligation are provided by a change in reaction conditions, namely the pH value. As it has been found that VyPAL2 activity is significantly influence by pH value, in particular on sites with D residues, said characteristic of VyPAL2 may be employed for bio-orthogonal (poly)peptide modification. Accordingly, in various embodiments, steps (i) and (ii) of the inventive methods are carried out at a first and a second pH-value that are different from each other, wherein the asparaginyl ligase, such as VyPAL2, has pH-dependent activity and specificity.

This means that in such methods, the binding and ligation site for an asparaginyl ligase at the C-terminus of the first (poly)peptide is preferably bound and ligated by the asparaginyl ligase at the first pH value and the binding and ligation site for an asparaginyl ligase at the C-terminus of either the second or third (poly)peptide is preferably bound and ligated by the asparaginyl ligase at the second pH value; or vice versa.

It has been found that VyPAL2 is an effective ligase for D-containing sites in the to-be-ligated peptide at comparably low pH values, such as a pH of about 6.0 and lower, for example in the range of 5.0 and lower, for example in the range of 3.5-6.0 or 3.5-5.0 or 4.0 to 5.0 or at about 4.5. In such embodiments, the N-residue of the binding and ligation sites disclosed for VyPAL2 above may be exchanged for D. Specifically, sites on which VyPAL2 has ligation activity at the described lowered pH values comprise those of the amino acid sequence (X)ₒDX³X⁴(X)ₚ, wherein X is any amino acid, o is an integer of at least 2, X³ is an amino acid selected from A, C, F, G, H, K, N, Q, R, S, Y, preferably G, S, N, Q and R, more preferably G or S, and X⁴ is a hydrophobic or aromatic amino acid, preferably selected from L, I, V, F, C, W, Y and M, preferably L, I, and F, more preferably L or F, and p is 0 or an integer of 1 or more. The preferred motif for VyPAL2 at this low pH values is (X)ₒDSL or (X)ₒDGF, in particular (X)ₒDSL. At pH values of above 6.0 the activity of VyPAL2 for such sites becomes significantly lower so that ligation is not effectively performed on these sites anymore.

In the described methods, the first pH value may therefore be a pH of about 6.0 or lower, for example in the range of 5.0 and lower, for example in the range of 3.5-6.0 or 3.5-5.0 or 4.0 to 5.0 or at about 4.5. The second pH value may be a pH of about 6.5 or higher, preferably a pH in the range of 6-5-7.4. a pH of above 7.4 is however not preferred. Alternatively, the first and second pH values may be exchanged such that the second pH value is a pH of about 6.0 or lower, and the first pH value is a pH of about 6.5 or higher.

In various embodiments, the binding and ligation site for an asparaginyl ligase at the C-terminus of the first (poly)peptide has the amino acid sequence (X)ₒDX³X⁴(X)ₚ, wherein X is any amino acid, o is an integer of at least 2, X³ is an amino acid selected from A, C, F, G, H, K, N, Q, R, S, Y, preferably G, S, N, Q and R, more preferably G or S, and X⁴ is a hydrophobic or aromatic amino acid, preferably selected from L, I, V, F, C, W, Y and M, preferably L, I, and F, more preferably L or F, and p is 0 or an integer of 1 or more, preferably (X)ₒDSL or (X)ₒDGF; and the binding an ligation site for an asparaginyl ligase at the C-terminus of the third (poly)peptide has the amino acid sequence (X)ₒNX³X⁴(X)ₚ, wherein X is any amino acid and o is an integer of at least 2, X³ is an amino acid selected from A, C, F, G, H, K, N, Q, R, S, Y, preferably G, S, N, Q and R, more preferably G or S, and X⁴ is a hydrophobic or aromatic amino acid, preferably selected from L, I, V, F, C, W, Y and M, preferably L, I, and F, more preferably L or F, and p is 0 or an integer of 1 or more, the preferred motif being (X)ₒNSL or (X)ₒNGF. In such embodiments, at low pH values the asparaginyl ligase binds and cleaves the D-containing motif and thus ligates the C-terminus of the first (poly)peptide to the N-terminus of the second (poly)peptide. The thus created ligation site is not acted upon if the pH is increased, since the affinity of the asparaginyl ligase for this site is significantly lower at higher pH values. At such higher pH values the third (poly)peptide is then added and the same asparaginyl ligase can the catalyze the ligation of the C-terminus of the third (poly)peptide to the N-terminus of the ligation product of the first step. While the order of steps can be reversed, this is not preferred, since even at low pH the asparaginyl ligase still has substantial activity for the N-containing site generated in the then first step and thus would cleave and ligate this site in a side reaction.

It is however possible in alternative embodiments of the described method that the D-containing binding and ligation site for an asparaginyl ligase is at the C-terminus of the second (poly)peptide; and the N-containing binding and ligation site for an asparaginyl ligase is at the C-terminus of the first (poly)peptide. In such embodiments, at low pH the C-terminus of the second (poly)peptide is then ligated to the N-terminus of the first (poly)peptide and after the pH has been increased, the C-terminus of the first (poly)peptide is ligated to the N-terminus of the third (poly)peptide. In such a setup, as the N-containing site is also being present at the low pH step, a side reaction of cleaving and ligating said N-containing site may occur.

In such embodiments, the binding an ligation site having the amino acid sequence (X)ₒD(X)ₚ is preferably bound to by the asparaginyl ligase at a pH of about 6.0 or lower, preferably a pH in the range of 4.5-6.0, and the binding an ligation site having the amino acid sequence (X)ₒN(X)ₚ is preferably bound to by the asparaginyl ligase at a pH of about 6.5 or higher, preferably a pH in the range of 6.5 to 7.4.

The inventors have further found that another asparaginyl ligases that can efficiently catalyze ligation for D-containing sites at low pH is OaAEP1b. In another aspect, the invention is thus directed to methods for (poly)peptide tandem ligation as described herein, comprising steps (i) and (ii) as defined above, wherein steps (i) and (ii) are carried out at a first and a second pH-value that are different from each other, wherein the first pH value is a pH of about 6.0 or lower, preferably a pH in the range of 4.5-6.0, and the second pH value is a pH of about 6.5 or higher, preferably a pH in the range of 6-5-7.4, wherein the first and second asparaginyl ligases are different and wherein the asparaginyl ligase used at a pH of about 6 or lower is OaAEP1b comprising or consisting of the amino acid sequence set forth in SEQ ID NO:44 or a variant thereof that has an amino acid sequence that has at least 80% sequence identity to the amino acid sequence set forth in SEQ ID NO:44 over its entire length and the asparaginyl ligase used at a pH of about 6.5 or higher is (i) VyPAL2 comprising or consisting of the amino acid sequence set forth in SEQ ID NO:1 or a variant thereof that has an amino acid sequence that has at least 80% sequence identity to the amino acid sequence set forth in SEQ ID NO:1 over its entire length or (ii) butelase-1 comprising or consisting of the amino acid sequence set forth in SEQ ID NO:2 and variants thereof that share at least 80% sequence identity with the amino acid sequence set forth in SEQ ID NO:2 over their entire length. In various embodiments of this method, step (i) may be the low pH step and step (ii) the higher pH step. The definition of variants given herein for VyPAL2 and butelase-1 similarly applies to OaAEP1b. Additionally, all embodiments described herein for the other methods of the invention similarly apply to this method.

In invention is further directed to methods for (poly)peptide cyclization. In such cyclization methods, no third (poly)peptide is used, but rather the second (poly)peptide already has a binding and ligation site for an asparaginyl ligase at its C-terminus. The two steps of the method therefore comprise ligating the second polypeptide to the N-terminus of the first (poly)peptide and then ligation the C-terminus of the first (poly)peptide to the N-terminus of the second (poly)peptide or reversing the order of ligations in that first the C-terminus of the first (poly)peptide is ligated to the N-terminus of the second (poly)peptide and then the C-terminus of the second polypeptide is ligated to the N-terminus of the first (poly)peptide. All the above-described embodiments with respect to asparaginyl ligases employed and binding and ligation sites on the peptides used are also applicable to these methods.

Specifically, these methods comprise the steps of:
(i) contacting a first (poly)peptide (A) having (i) at its C-terminus a binding and ligation site for an asparaginyl ligase with a second (poly)peptide (B) to be ligated to said first (poly)peptide having at its C-terminus a binding an ligation site for an asparaginyl ligase and a first asparaginyl ligase (C) under conditions that allow ligation of the second (poly)peptide to the C- or N-terminus of the first (poly)peptide to yield a modified first (poly)peptide; and
(ii) contacting the modified first (poly)peptide obtained in step (i) with a second asparaginyl ligase (E) under conditions that allow ligation of the C-terminus of the modified first (poly)peptide to its N-terminus to yield a cyclized first (poly)peptide.

In these cyclization methods, the first and second asparaginyl ligase are different and are each selected from (a) VyPAL2 comprising or consisting of the amino acid sequence set forth in SEQ ID NO:1 and variants thereof that share at least 80% sequence identity with the amino acid sequence set forth in SEQ ID NO:1 over their entire length, and (b) butelase-1 comprising or consisting of the amino acid sequence set forth in SEQ ID NO:2 and variants thereof that share at least 80% sequence identity with the amino acid sequence set forth in SEQ ID NO:2 over their entire length, such that the first asparaginyl ligase is VyPAL2 or a variant thereof and the second is butelase-1 or a variant thereof or vice versa.

In various embodiments of the methods described herein, the terminus of one of the (poly)peptides employed in a given method step may be blocked to prevent its ligation in this step. Said block may be removed for a subsequent step to also allow the ligation of this previously blocked terminus. Said blocked terminus may be the N-terminus of the first (poly)peptide. Said blocking of the N-terminus may be achieved by using an N-terminal cysteine residue that forms a thiazolidine cap with glyoxylic acid. Said cap may be removed by using silver ions (Ag⁺) and thus make the N-terminal end available for another ligation step.

In the methods described herein, at least one of the (poly)peptides to be ligated may be further conjugated to an organic moiety. For this purpose, the (poly)peptide may comprise a reactive group, typically not at the terminus to be ligated. Said reactive group, which may also be a side chain of an amino acid, may then be conjugated to an organic moiety of interest in a further step of the method. The organic moiety may be any molecule or group and comprises pharmaceutically active agents and detectable markers, such as fluorescent markers or biotin. In various embodiments, the active agent may be a small organic molecule pharmaceutical, such as a cancer therapeutic agent, including, but not limited to an anthracycline, such as doxorubicin.

In further aspects of the invention, also encompassed are uses of the two asparaginyl ligases described herein for the production or synthesis of dually modified (poly)peptides by protein tandem ligation. Said uses may comprise contacting a first (poly)peptide (A) having at its C-terminus a binding and ligation site for an asparaginyl ligase with a second (poly)peptide (B) to be ligated to said first (poly)peptide and a first asparaginyl ligase (C) under conditions that allow ligation of the second (poly)peptide to the C-or N-terminus of the first (poly)peptide to yield a modified first (poly)peptide; and contacting the modified first (poly)peptide obtained in step (i) with a third (poly)peptide (D) to be ligated to said modified first (poly)peptide and a second asparaginyl ligase (E) under conditions that allow ligation of the third (poly)peptide to the C- or N-terminus of the first (poly)peptide to yield a dually modified first (poly)peptide.

In any case, all embodiments disclosed above for the methods according to the invention are similarly applicable to these uses.

The (poly)peptides to be ligated or cyclized according to the methods and uses disclosed herein can be fusion peptides or polypeptides in which an Asx-containing tag has been C-terminally fused to the (poly)peptide of interest that is to be ligated or fused. The Asx-containing tag preferably has the amino acid sequences of the binding and ligation site for asparaginyl ligases defined above, including the various embodiments. Generally, polypeptides and proteins that may be ligated to peptides, such as peptides bearing signaling or detectable moieties, or cyclized using the methods and uses described herein, include, without limitation antibodies, antibody fragments, antibody-like molecules, antibody mimetics, peptide aptamers, hormones, various therapeutic proteins and the like.

In various embodiments, the ligase activity is used to fuse a peptide bearing a detectable moiety, such as a fluorescent group, including fluoresceins, such as fluorescein isothiocyanate (FITC), or coumarins, such as 7-amino-4-methylcoumarin, to a polypeptide or protein, such as those mentioned above. In various embodiments, the protein can be an antibody fragment or an antibody mimetic.

Detectable markers useful in the methods and uses of the invention include fluorescein or derivatives thereof and/or a peptide that can easily be radiolabeled with elements I-125 or I-131, since this allows using a single reagent imaging of tumors in vivo using PET or SPECT followed by fluorescent detection in organ sections or biopsies.

In the methods and uses described herein, the enzyme, i.e. the asparaginyl ligases, and the substrates, i.e. the first, second and optional third (poly)peptide, can be used in a molar ratio of 1:100 or higher, preferably 1:400 or higher, more preferably at least 1:1000.

The reaction is typically carried out in a suitable buffer system at a temperature that allows optimal enzyme activity, usually between ambient (20° C.) and 40° C.

Immobilizing enzymes on solid supports has a long history with a primary goal of lowering enzyme consumption by repetitively using the same batch of enzymes. In addition, site-separation of solid-phase immobilization reduces aggregation, leading to increased stability and activity of biocatalysts, and simplifies the purification by avoiding contamination of products by enzymes. Consequently, immobilized biocatalysts have been developed for industrial uses to a billion-scale market, such as immobilized lactase in food industry and immobilized lipase in biodiesel production. Compared with conventional industrial processes using chemical catalysts, immobilized enzymes are economically attractive and environmentally friendly. There are three main-stream immobilization technologies, including attachment to carriers either or non-covalently, physical entrapment, and self-crosslinking. For biocatalysts such as the PALs described herein that have an exposed substrate-binding surface for biomolecule-based substrates, strategies based on attachment to hydrophilic porous resins by either covalent-binding and affinity-binding methods are direct, convenient, and feasible to facilitate their performance in aqueous conditions. The thus immobilized asparaginyl ligases are stable, reusable and highly efficient in mediating macrocyclization and site-specific ligation reactions.

Accordingly, in the methods of the present invention the first and/or second asparaginyl ligases may be immobilized on a solid support. The major advantages of immobilization on a solid support provide site separation and pseudo-dilution to prevent trans-autolytic degradation and enhance stability. Site-separation of immobilized enzymes permits the use of high enzyme concentrations to accelerate ligation reactions to complete in minutes, such as cyclization, cyclooligomerization and ligation reactions either under one-pot conditions or in a continuous flow-reactor. Suitable support materials include various resins and polymers that are used in chromatography columns and the like. The support may have the form of beads or may be the surface of larger structure, such as a microtiter plate. Immobilization allows for a very easy and simple contacting with the substrate, as well as easy separation of enzyme and substrate after the synthesis. If the polypeptide with the enzymatic function is immobilized on a solid column material, the ligation/cyclization may be a continuous process and/or the substrate/product solution may be cycled over the column.

In various embodiments, the asparaginyl ligase is glycosylated and the immobilization is facilitated by interaction with a carbohydrate-binding moiety, preferably a concanavalin A moiety or variant thereof, covalently linked to the solid support. In such embodiments, the solid support may be an agarose bead.

In various other embodiments, the asparaginyl ligase is biotinylated and the immobilization is facilitated by interaction with a biotin-binding moiety, preferably a streptavidin, avidin or neutravidin moiety or variant thereof, covalently linked to the solid support. Functionalization of the enzyme with the biotin may be achieved using methods known in the art, such as functionalization with a biotin ester with N-hydroxysuccinimide (NHS), such as succinimidyl-6-(biotinamido)hexanoate. In such embodiments, the solid support may be an agarose bead and the biotin-binding moiety may be an avidin variant, such as neutravidin (deglycosylated avidin).

In various other embodiments, the asparaginyl ligase is immobilized on the solid support by reaction of free amino groups in the polypeptide, for example from lysine side chains, with an N-hydroxysuccinimide functional group on the surface of the solid support. The solid support may be agarose beads.

In all these embodiments, the asparaginyl ligases may be the butelase-1 and variants thereof of VyPAL2 and variants thereof, as described herein.

The invention is further illustrated by the following non-limiting examples and the appended claims.

### EXAMPLES

### Materials and Methods

All amino acids, coupling reagents, solvents and resins were purchased from Sigma and Chemimpex. All solvents and reagents were used as received without further purification. VyPAL2 and butelase-1 were prepared as previously described (WO 2020/226572 A1).

HPLC. Analytical RP-HPLC was run on a SHIMADZU (Prominence LC-20AT) instrument using an analytical column (Grace Vydac “Protein C4”, 250×4.6 mm, 5 μm particle size) at a flow rate of 1.0 mL/min. Analytical HPLC elution was monitored by UV absorption at 214 nm and 254 nm. Semi-preparative RP-HPLC was run on a SHIMADZU (Prominence LC-20AT) instrument using a semi preparative column (Grace Vydac “Protein C4”, 250×10 mm, 10 μm particle size) at a flow rate of 2.5 mL/min. Both analytical and semi-preparative HPLC were run at room temperature using a gradient of solvent B in solvent A. Solvent B was 90% acetonitrile in water (0.040% TFA) and solvent A was water (0.045% TFA). Both solvents were filtered through 0.22 μm filter paper and sonicated for 30 min before use.

Protein expression and purification. Genes encoding the desired protein sequences were cloned into pETDuet vector and the plasmids were then transformed into E. coli BL21 (DE3) competent cells by the standard 90 s heat shock protocol. The bacterial colonies were then picked up and transferred to liquid LB medium in a culturing flask. The flask was shaken in the incubator at 37° C. for 8-12 h until the OD reached 0.6-0.8, followed by induction with 1 mM of IPTG at 37° C. in 4-8 h for protein expression. Cells were harvested and lysed by sonication in lysis buffer containing 50 mM sodium phosphate and 500 mM NaCl (pH=8.0). After centrifugation, the supernatant was loaded on a column of Ni-NTA beads and incubated at 4° C. for 1 h. The beads were washed 3 times with the lysis buffer and the protein was subsequently eluted with lysis buffer containing 250 mM imidazole. The purified protein was dialyzed in phosphate buffer (pH=6.5) overnight and stored in the freezer at −20° C.

Mass spectrometry. ESI mass spectrum data of small peptides and proteins were obtained from a Thermo Finnigan LCQ DECA XP MAX (ESI ion source, positive mode). The software of MagTran 1.03 and ESIProt 1.0 were used for the data deconvolution.

Tissue culture and cell imaging. Cells were maintained in 10% FBS in DMEM (high glucose) at 37° C. in an incubator under 5% CO₂. For passaging, cells were first washed 3 times with trypsin-EDTA (0.25%) to detach the cells from tissue culture plates. Then a 3-times volume of complete DMEM medium was added to neutralize trypsin activity. Cells were grown till 40-60% confluency. Peptides or proteins in complete medium were applied to the cells and incubated for 30 min at 37° C. Washing was done 3 times with PBS and cells were subsequently subjected to microscopy analysis.

Cell viability assay. MTT assays were carried out following recommended protocols from Sigma-Aldrich (Cat. No. 11465001001). Cells were first seeded in a 96-well tissue culture plate with 100 μl medium to grow until the confluency reached 40-60% of the plates surface. Peptides and proteins were added and incubated for 84 h, followed by adding 10 μl of MTT I to each well and further incubated for approximately 4 h. After this, MTT II was added and incubated at 37° C. for overnight to solubilize the purple crystals. Spectrophotometrical absorbance measurement of the samples was carried out by using a microplate reader (Biotek, citation 5) at the wavelength of 575 nm and the reference wavelength was 670 nm.

Cell staining and imaging. MCF-7 and A431 cells cultured in 24 well plates were washed with PBS for 3 times. Formaldehyde (4%, w/v in PBS) was then added to each well for 15 min to fix the cells. After that, the cells were washed again with PBS for 3 times to remove the residual formaldehyde. To permeabilize the cell, Triton X-100 (0.1%, w/v in PBS) was added to the wells for 5 min, and then PBS was used to wash the cells for 3 times before subjecting to staining. To stain the cells, doxorubicin, protein 20 or 48 and DAPI were diluted in PBS to the concentration of 10 μM, 2 μM and 700 nM, respectively. Then the solution was added to each well for 30 min. After this, the cells were washed with PBS for 3 times and subjected to imaging analysis using Inverted Fluorescence Microscope (Olympus Life Science #IX71). To acquire DAPI fluorescent image, the “Blue” channel (Filter Cube: 350 nm) was used. Likewise, “Red” channel (Filter Cube: 550 nm) was used to obtain the doxorubicin fluorescence and “Green” channel (Filter Cube: 450 nm) for fluorescein.

Solid phase peptide synthesis. All the peptides used in this study were synthesized as C-terminal amides using Rink amide MBHA resin by standard Fmoc chemistry. Before use, the resin was pre-swelled in DMF for 20 min. Before the first coupling, an Fmoc deprotection procedure was performed using 20% piperidine in dimethylformamide (DMF) for 30 min. The resin was then washed with DMF, DCM and DMF successively. For the coupling reactions, 3 eq. of FmocAA-OH, 3 eq. of PyBOP were first dissolved in DMF/DCM (1:1). The mixture was added to the resin, followed by the addition of 6 eq. of DIEA. Coupling reactions were carried out for 60 to 90 min. Coupling efficiency was examined by ninhydrin test. The peptides were cleaved from the resin with a cocktail containing 95% TFA, 2.5% water and 2.5% TIS for 2 h. After precipitation with cold diethyl ether, the crude peptides were purified using HPLC. Desired peptides were obtained in the powder form after lyophilization. All peptides were characterized by electrospray ionization mass spectrometry.

List of peptides prepared (amino acids are represented by single-letter codes (in bold) except for citrulline which is represented as Cit; letters in lower case denote D-amino acids; PABC=para-aminobenzyloxycarbonyl; Dox=doxorubicin):

Peptide 1 (SEQ ID NO: 7):
Ac-KKLAVINHV;
1061.01 (observed), 1062.27 (calculated).
Peptide 2 (SEQ ID NO: 8):
GIGGIKA;
613.68 (observed), 613.74 (calculated).
Peptide 4 (SEQ ID NO: 9):
YKANGL;
664.26 (observed), 664.67 (calculated).
Peptide 5 (SEQ ID NO: 10):
GFGGIKA;
648.38 (observed), 648.52 (calculated).
Peptide 7 (SEQ ID NO: 11):
Ac-KKLAVINGF;
1031.34 (observed), 1031.56 (calculated).
Peptide 9 (SEQ ID NO: 12):
Fluorescein-GRANGI;
944.52 (observed), 944.97 (calculated).
Peptide 11 (SEQ ID NO: 13):
GIGGFKGG-klaklakklaklak;
2197.07 (observed), 2197.72 (calculated)
(lower case aa = D-amino acids).
Peptide 15 (SEQ ID NO: 14):
GFLGVK(COCH₂ONH₂)ANHV;
1113.90 (observed), 1113.29 (calculated).
Peptide 21a (SEQ ID NO: 15):
GISTKSIPPISYRDGL;
1703.18 (observed), 1701.94 (calculated).
Peptide 21b (SEQ ID NO: 16):
GISTKSIPPISYRDDL;
1761.11 (observed), 1759.95 (calculated).
Peptide 21c (SEQ ID NO: 17):
GISTKSIPPISYRDAL;
1717.14 (observed), 1715.96 (calculated).
Peptide 21d (SEQ ID NO: 18):
GISTKSIPPISYRDLL;
1759.00(observed), 1758.00 (calculated).
Peptide 21e (SEQ ID NO: 19):
GISTKSIPPISYRDSL;
1733.12 (observed), 1731.95 (calculated).
Peptide 21f (SEQ ID NO: 20):
GISTKSIPPISYRDRL;
1802.27 (observed), 1801.02 (calculated).
Peptide 21g (SEQ ID NO: 21):
GISTKSIPPISYRDKL;
1774.25 (observed), 1773.01(calculated).
Peptide 21h (SEQ ID NO: 22):
GISTKSIPPISYRDQL;
1774.14(observed), 1772.98(calculated).
Peptide 21i (SEQ ID NO: 23):
GISTKSIPPISYRDEL;
1775.98 (observed), 1773.96 (calculated).
Peptide 21j (SEQ ID NO: 24):
GISTKSIPPISYRNGL;
1701.94 (observed), 1700.96 (calculated).
Peptide 26: GVCit-PABC-fluorescein;
881.65 (observed), 880.35 (calculated).
Peptide 28 (SEQ ID NO: 25):
GIGGIRK(fluorescein);
1057.65 (observed), 1057.35 (calculated).
Peptide 30: GVCit-PABC-Dox;
1006.36 (observed), 1005.40 (calculated).
Peptide 34a (SEQ ID NO: 26):
Ac-QRLGNQWAVGHLMGSGSDSL;
2154.04 (observed), 2153.04 (calculated).
Peptide 34b (SEQ ID NO: 27):
Ac-IHGHHIISVGGSGSDSL;
1713.82 (observed), 1713.88 (calculated).
Peptide 34c (SEQ ID NO: 28):
Ac-VPWMEPAYQRFLGSGSDSL;
2482.1 (observed), 2180.04 (calculated).
Peptide 34d (SEQ ID NO: 29):
Ac-YHWYGYTPQKVIGSGSDSL;
2200.00 (observed), 2199.41 (calculated).
Peptide 34e (SEQ ID NO: 30):
Ac-CMYIEALDKYACGSGSDSL;
2068.83 (observed), 2065.88 (calculated).
Peptide 34f (SEQ ID NO: 31):
Ac-CRGDRGDCGSGSDSL;
1525.76 (observed), 1524.60 (calculated).
Peptide 37 (SEQ ID NO: 32):
Fluorescein-GRADGI;
944.40 (observed), 943.38 (calculated).
Peptide 41 (SEQ ID NO: 33):
GIAAK(Ac);
500.50 (observed), 499.31 (calculated).
Peptide 44 (SEQ ID NO: 34):
YKANGL;
633.45 (observed), 633.78(calculated).

### Example 1

### Differential Substrate Specificity of Butelase-1 and VyPAL2 Analyzed by Kinetic Studies

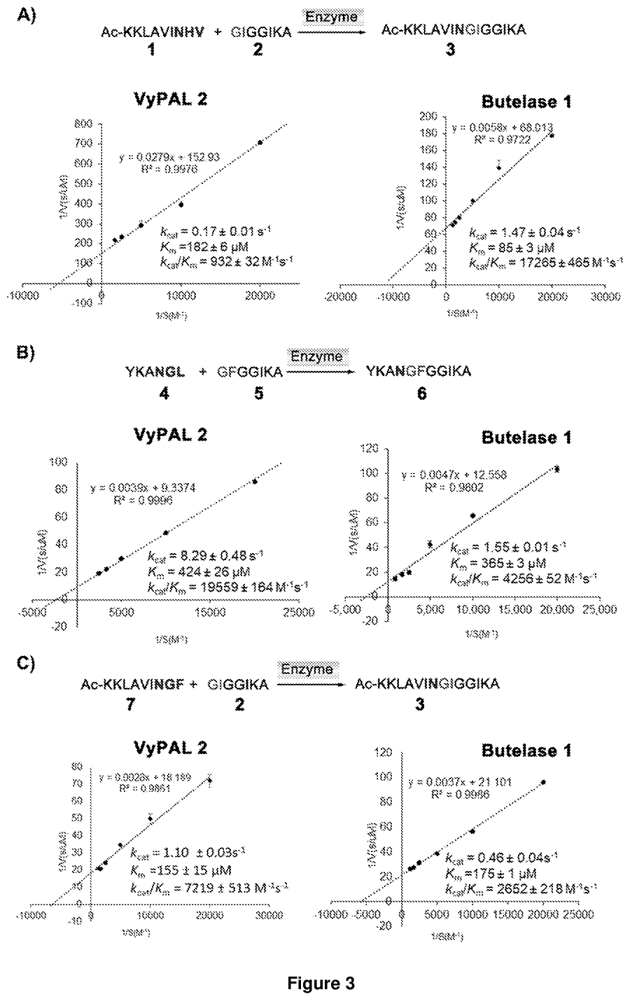

PAL enzymes have been used extensively for protein single-site labeling and macrocyclization. However, using two PALs of different substrate specificity for bio-orthogonal and dual ligation remains unexplored. Previous work has revealed some noticeable differences in substrate specificity between butelase-1 and VyPAL2 [25, 42]. To determine the differences quantitatively, firstly the kinetics of VyPAL2 and butelase-1 toward peptide 1 or 7 which has a C-terminal NHV or NGF tripeptide motif respectively were studied (Table 1). The nucleophile substrate, used at a constant concentration, was peptide 2 which contains an N-terminal GI dipeptide motif. Reverse-phase analytical HPLC was used to monitor and quantify the ligation reaction. The results showed that the catalytic activity of butelase-1 towards the acyl peptide substrate 1 was about 18 times that of VyPAL2, whereas the catalytic activity of VyPAL2 towards substrate 7 was about 3 times of that of butelase-1 (Table 1, FIG. 3). Similarly, kinetics of the two ligases towards a GF-starting nucleophile substrate, peptide 5, were also examined.

In this case, the acyl substrate, now kept at a constant concentration for the kinetic study, was peptide 4 which contains NGL at the C-terminus, a favorable motif for both VyPAL2 and butelase-1. It was shown that VyPAL2 has 5-fold catalytic efficiency than butelase-1 towards the GF-peptide substrate 5 (Table 1 and FIG. 3). In summary, the kinetic studies confirm the differential activities of butelase-1 and VyPAL2 toward certain substrate sequences, which provide strong support for a two-PAL, bio-orthogonal tandem ligation scheme for protein dual labeling.

TABLE 1
Kinetics of VyPAL2- and butelase-1-mediated intermolecular ligation.

| Electrophile | Nucleophile | | | | k_{cat}/Kₘ |
|---|---|---|---|---|---|
| substrate | substrate | Enzyme | k_{cat} [S⁻¹] | Kₘ [μM] | [M⁻¹S⁻¹] |
| Ac-KKLAVINHV | GIGGIKA | VyPAL2 | 0.17 ± 0.01 | 182 ± 6 | 932 ± 32 |
| 1 | 2 | Butelase-1 | 1.47 ± 0.04 | 85 ± 3 | 17265 ± 465 |
| YKANGL | GFGGIKA | VyPAL2 | 8.29 ± 0.48 | 424 ± 26 | 19559 ± 164 |
| 4 | 5 | Butelase-1 | 1.55 ± 0.01 | 365 ± 3 | 4256 ± 52 |
| Ac-KKLAVINGF | GIGGIKA | VyPAL2 | 1.10 ± 0.03 | 155 ± 15 | 7219 ± 513 |
| 7 | 2 | Butelase-1 | 0.46 ± 0.04 | 175 ± 1 | 2652 ± 218 |

### Example 2

### Applying the Two PAL-Based Bio-Orthogonal Tandem Ligation Method for Affibody Dual Labeling

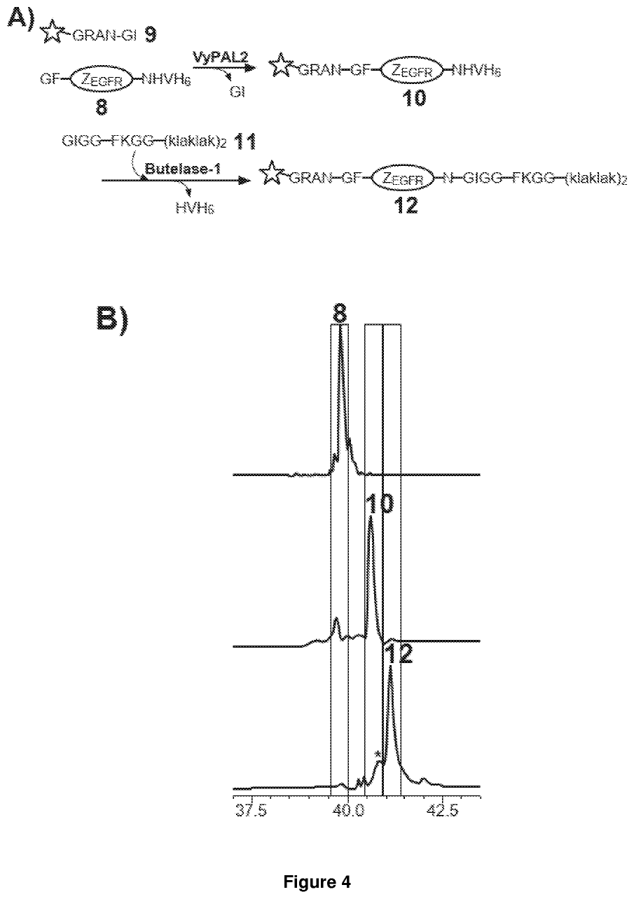

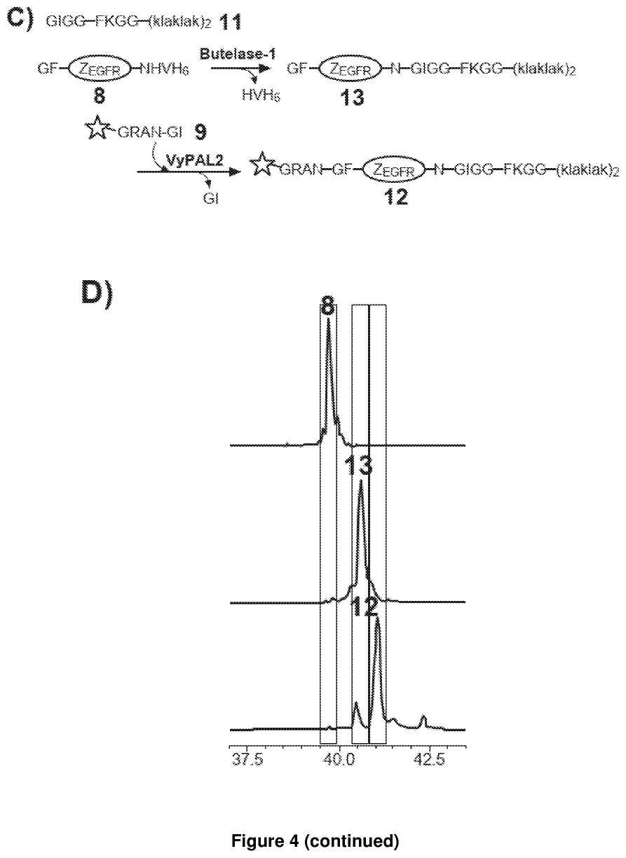

Butelase-1 and VyPAL2 were used to dually label an affibody through tandem enzymatic ligation (FIG. 4). Considering the specificity of the two enzymes, an N-terminal GF dipeptide tag and a C-terminal NHV tripeptide tag were introduced onto ZEGFR to give 8. A new fluorescein-peptide 9 with a C-terminal NGI motif was prepared. Also synthesized was peptide 11 of the sequence GIGGFKGG-klaklakklaklak (SEQ ID NO:13) of which the all-D amino-acid sequence is the mitochondrion-lytic KLA peptide [52]. Phe-Lys is a cathepsin B-sensitive linker and so can be cleaved in the lysosomes to release the KLA peptide. 9 and 11 were used to label the respective N- and C-termini of the ZEGFR 8. Sequential bio-orthogonal ligations were conducted in both N-to-C (FIG. 4A) and C-to-N (FIG. 4C) directions. For N-to-C tandem ligation, VyPAL2 was used at the first ligation step and butelase-1 at the second step (FIG. 4A). C-to-N sequential ligations were performed using the two enzymes in the reverse order (FIG. 4C). Whichever the ligation direction, the same final product 12 was obtained, as characterized by ESI-MS (obs: 10776, calc: 10773). The reactions at each step of the two schemes were remarkably clean with good conversion yields. In N-to-C ligation, 50 μM of Z_{EGFR} 8 and 250 μM of peptide 9 were first reacted in the presence of 150 nM of VyPAL2 at 37° C. for 30 min. The reaction gave ca. 80% of product 9 based on HPLC analysis. After HPLC purification and refolding, the second step was performed by incubating 50 μM of 10 and 250 μM of peptide 11 with 100 nM of butelase-1 for 20 min at 37° C. The second step gave about 70% conversion yield (FIG. 4B). This was due to that, although the newly formed NGF motif in 10 is not a favored substrate of butelase-1, it could still be affected in BML which resulted in the cleavage of the N-G bond for transpeptidation with 11. In C-to-N ligation, BML was first performed by mixing 50 μM of ZEGFR 8 and 250 μM of peptide 11 with 100 nM of butelase-1 at 37° C. for 30 min to afford 13 in about 85% based on HPLC analysis. Then, VML was performed by incubating 50 μM of 13 and 250 μM of peptide 9 with 100 nM of VyPAL2 at 37° C. for 30 min. The reaction gave 12 in ˜70% (FIG. 4D). It is worth mentioning that, in the C-to-N scheme, the NGI sequence formed at the first BML step was not affected significantly when conducting VML, likely because 9 was used in 5-fold excess to 13 and the C-terminal NGI in 9 is more exposed than the NGI sequence in 13. Also, the free ^{α}N-amino group of the N-terminal Gly residue in the affibody was resistant to butelase-1 at the first ligation step.

While it would be ideal to use an incoming nucleophile peptide sequence at the first BML step that would generate a site that is sub-optimal for recognition by VyPAL2, when testing peptides with N-terminal HV or GV dipeptide motif for BML in the first step, these were found to be poorer nucleophile substrates than the GI-peptides for butelase-1 recognition (data not shown). Therefore, although using an NH-peptide could make the C-to-N scheme a potentially more orthogonal method, such scheme would be significantly less efficient than the one using a GI-peptide.

Because the two PALs require only a short NXY tripeptide as the recognition tag and ligate at the Asn residue, only minimal traces are left in the modified proteins. These results show the robustness and neatness of the sequential bio-orthogonal ligation method for protein dual labeling.

### Example 3

### Binding Affinity (KD) of the Dual Labeled Affibody 11 to EGFR on A431 Cells

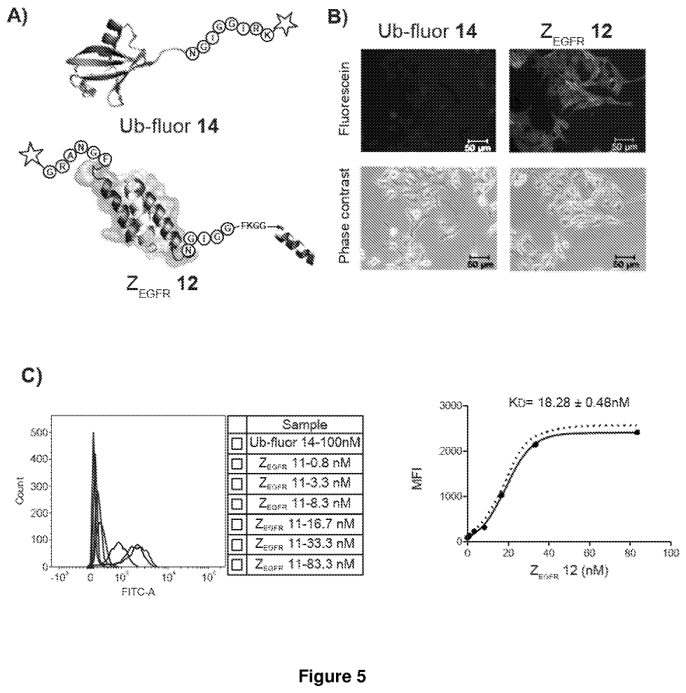

To study the activities of the dually labeled product 12, its binding towards EGFR-overexpressing A431 cells was analyzed. The cells were treated with 100 nM of 12 while the fluorescein-tagged ubiquitin 14, which was prepared via BML with the fluorescent peptide was used as a negative control. As shown in FIG. 5A, strong green fluorescence was observed in A431 cells treated with 12 (FIG. 5B), while no fluorescence was seen in A431 cells treated with 14. Meanwhile, FACS analysis indicated a remarkable shift of the fluorescence intensity for the 12-treated cells in reference to ubiquitin 14-treated cells in the control group (FIG. 5C), which was consistent with the fluorescent imaging data. To determine the dissociation constant (KD) of 12, these cells were treated with different concentrations of 12 and subjected to FACS analysis after 30 min of incubation. As shown in FIG. 5C, treatments with different concentrations of 12 resulted in different intensity shifting. The Mean Fluorescent Intensity was analyzed in non-linear regression function, which gave the K_{D}=18.28±0.48 nM.

### Example 4

### Cytotoxicity Evaluation of the Dual-Labeled affibody

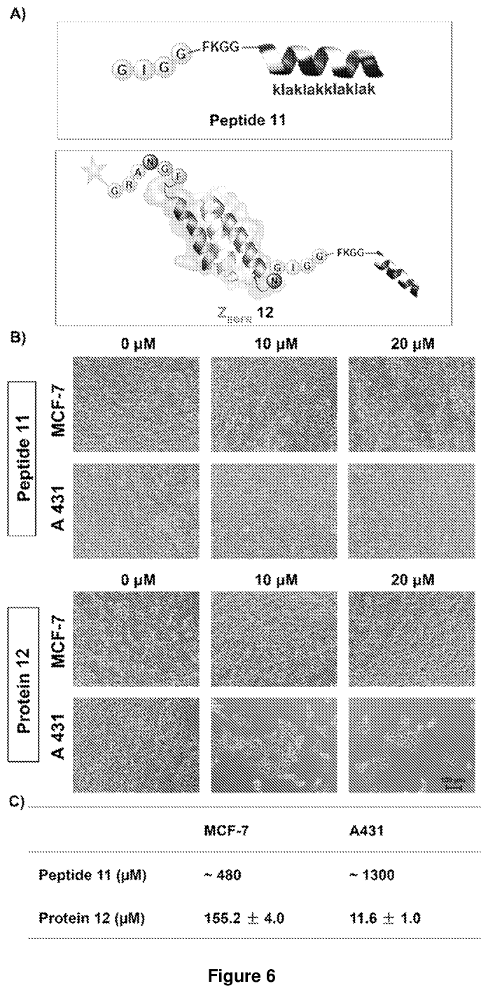

An MTT assay was performed to determine whether 12 had any effects on the two cell lines, the EGFR-overexpressing A431 cells and the MCF-7 cells which have a low EGFR expression level [54]. Both cell lines were treated with 12 for 84 h and then subjected to MTT analysis. 12 exhibited significant toxicity to A431 cells with an IC₅₀ of 11.6±1.0 μM, whereas it showed an IC₅₀ of 155.2±4.0 μM for MCF-7 cells. The unconjugated peptide 11 had an IC₅₀ of about 480 μM and 1300 μM against MCF-7 and A431 cells, respectively (FIG. 6A, B). Owing to its poor cellular uptake [55], the low cytotoxicity of 11 itself in the two cell lines is not unexpected [52]. However, conjugating the peptide to the EGFR-targeting affibody drastically enhanced its cytotoxicity against A431 cells, likely because the affibody helped deliver the mitochondrion-lytic peptide intracellularly via EGFR-mediated endocytosis. So, as the internalized 12 ended up in the lysosomes, the high proteolytic activity by enzymes like cathepsin B would destroy the peptide linker and even the affibody to release the KLA D-peptide which, after escaping from lysosomes, would disrupt the mitochondrial membrane, leading to apoptosis.

The above data clearly show that a protein with orthogonal N- and C-terminal recognition tags can be dually labeled by the consecutive action of two PALs with differential substrate specificities. The dually labeled affibody protein has selective imaging and cytotoxic activities. To further demonstrate the versatility of the tandem ligation scheme, the synthesis of a cyclic form of the affibody tagged with doxorubicin was carried out (FIG. 2; Example 5).

### Example 5

### Synthesis of a Cyclic Affibody-Doxorubicin Conjugate

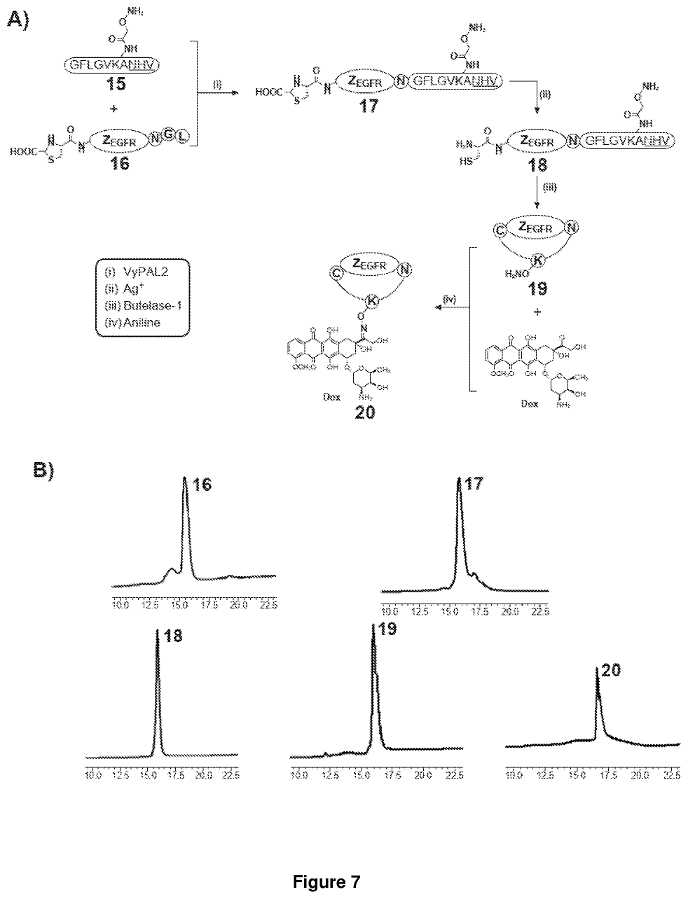

For this purpose, peptide 15 containing an N-terminal GF dipeptide as the nucleophile substrate for VyPAL2 and a C-terminal NHV tripeptide motif at the C terminus as the electrophile substrate for butelase-1 was prepared using SPPS (FIG. 7A). The aminooxy functional group in the peptide would allow conjugation with DOX through its ketone group by oxime formation [56]. For obvious reasons, 8, the affibody substrate for dual labeling, could not be used here because the inventing peptide 15 already contains the same respective nucleophile and electrophile substrates for the two PALs (FIG. 7A). Therefore, affibody ZEGFR 16 containing “CG-” at the N terminus and “-NGL” at the C-terminal end was prepared recombinantly in E. coli. Interestingly, ESI-MS analysis showed that the N-terminal cysteine of 16 was capped, during protein expression, presumably as a thiazolidine moiety by the ubiquitous aldehyde metabolite glyoxylic acid in the bacterial cells, effectively blocking it from being used as a nucleophile substrate by the PAL enzymes. Thus, only the C-terminal labeling product Z_{EGFR} 17 would be generated in the first ligation step without possibility of cyclization or self-ligation of 17. As expected, when VML was performed by mixing 50 μM Z_{EGFR} 16 and 150 μM peptide 15 with 100 nM of VyPAL2 at 37° C. for 30 min, only the C-terminal ligation product 17 was obtained as clearly shown by HPLC and ESI-MS analysis (FIG. 7B). The NHV tag in 15 or 17 was not affected, confirming again its orthogonality toward VyPAL2.

To unmask the N-terminal cysteine in 17, 17 (1 mM) was treated with silver nitrate (10 mM) for 30 min, followed by treatment with β-mercaptoethanol (100 mM) for 30 min. The deprotection reaction gave product 18 as confirmed by HPLC and ESI-MS (FIG. 7A, 7B). Butelase-mediated cyclization was performed by mixing 18 (100 μM) with 50 nM of butelase-1 for 30 min at 37° C. The cyclic product 19 was characterized by HPLC and ESI-MS (FIG. 7B). The aminooxy functional group in ZEGFR 19 can react with the doxorubicin ketone group through Schiff's base formation. So the oxime ligation reaction was carried out by mixing 100 μM of Z_{EGFR} 19 and 1 mM of Dox in the presence of 10 mM of aniline as the catalyst at pH 6 and 37° C. for overnight. The reaction gave the final product 20 as characterized by HPLC and ESI-MS (FIG. 7B).

### Example 6

### Cell Imaging and Cytotoxic Study of the Synthesized Cyclic Affibody-Doxorubicin Conjugate 20

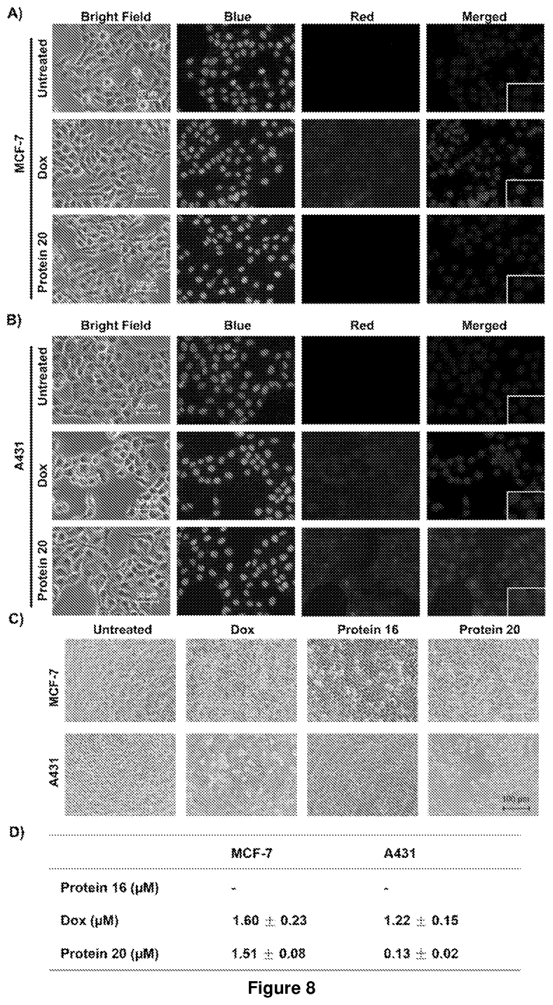

Fluorescence imaging, microscopy analysis and MTT assay were performed to determine the binding and inhibitory effects of the cPDC 20 on MCF-7 and A431 cell lines. The intrinsic fluorescence of doxorubicin serves as an imaging tool to visualize the binding of 20 to the cells (FIG. 8A, 8B). As shown in the figures, only the EGFR-overexpressing A431 cells were positively stained after a 30-min treatment with 20. The same treatment did not yield any staining on the EGFR-negative MCF-7 cells.

On the other hand, both cell lines were stained by the free doxorubicin, which is not surprising as it can enter cells and bind to nuclear DNA. In the cytotoxicity experiments, both cell lines were treated with 0.2 μM of unconjugated affibody 16, doxorubicin and 20 for 96 h and subjected to microscopy analysis. At this concentration, 20 exhibited substantial cytotoxic effect on A431 cells, with smaller or no effects observed in the other control settings (FIG. 8C). Next, MTT assay was conducted to determine the IC₅₀. The unconjugated affibody 16, Dox and cPDC 20 were added at varying concentrations to MCF-7 and A431 cells for 96 h. As seen from FIG. 8D, 20 showed significantly higher toxicity with an IC₅₀ of 0.13±0.02 μM to A431 cells than to MCF-7 cell (IC₅₀=1.51±0.08 μM). The affibody itself had little cytotoxic effect even at very high concentrations, which is consistent with previously published results [58]. The unconjugated DOX exibited lower cytotoxicity in terms of IC₅₀ to A431 cells compared to 20, likely due to a lack of receptor-mediated enrichment of the compound in the cells. The measured IC₅₀ of Dox in MCF-7 and A431 was 1.60±0.23 μM and 1.22±0.15 μM, respectively (FIG. 8D). The enhanced toxicity of the cylcoaffibody-DOX conjugate 20 was likely due to the fast enrichment of the conjugate via receptor-mediated endocytosis which would uptake the conjugate through the endosomal pathway and deliver it to the lysome. The acidic milieu in this organelle would help cleave the oxime

linkage to release DOX [56]. Attributing to its hydrophobic property, doxorubicin could easily escape from lysosome to bind to nuclear DNA, leading to apoptotic cell death.

### Example 7

### pH-Dependent Catalytic Activity of Butelase-1, VyPAL2 and OaAEP1b Towards Aspartyl and Asparaginyl Substrates

pH plays a critical role in determining the catalytic behaviors of AEPs [34, 38]. At acid pH (pH 4-5), most AEPs function as hydrolases to cleave asparaginyl or aspartyl peptide bonds. This is also one of their natural functions in the acidic environment of the vacuoles where they process the large vacuolar protein precursors, including their own ones, to their mature forms [34, 38, 42, 59]. As pH increases, AEPs gradually lose their activity towards aspartyl peptide bonds because of weakened substrate binding resulting from the loss of a hydrogen bond donor from the hydroxyl of γCOOH of the P1-Asp [40, 60, 61, 66,] which is important for interacting with a key residue in the enzymes' S1 pocket. So at near neutral to basic pH, APEs are often completely inactive against aspartyl substrates. For asparaginyl peptide substrates, their binding to AEPs is not so much affected by pH changes since the amide protons on the Asn sidechain amide remain available for hydrogen binding at higher pH. So AEPs are catalytically active against asparaginyl peptide bonds for their hydrolytic cleavage in a wide pH range (from acidic to weakly basic). Obviously, an increase of pH also makes the amine nucleophile in an acyl acceptor substrates more available in a ligation reaction. As a result, the transpeptidation (i.e., ligation) activity of many AEPs also increases with the increase of pH. The ratio of ligation versus hydrolysis activity depends on the nature of the substrates (sequence and conformation) [25, 33, 35, 61] and the AEP itself [36, 37, 42, 59, 66]. While a large number of AEPs exhibit a bifunctional profile of dual hydrolytic and ligation activity which is pH- and/or substrate-dependent, some are predominantly proteases whatever the pH [25, 35, 42, 59, 66]. For these protease-AEPs, hydrolysis always prevails even for those substrates that are prone for cyclization [31, 42, 59]. For the bifunctional AEPs, ligation can predominate hydrolysis in the cases of entropy-favorable reactions where the reacting partners are positioned in close proximity, such as in certain intramolecular (i.e., cyclization) or conformation-assisted intermolecular ligations [35, 62, 63]. On the other hand, a few members of the AEP family function almost exclusively as ligases as they are essentially devoid of any hydrolase activity at near-neutral or mildly acidic pH as long as a reacting nucleophile is present, which qualifies them as pure peptide asparaginyl ligases or PALs. Examples of naturally existing PALs include butelase-1 and VyPAL2. Butelase-1, the flagship PAL, is also the most efficient among the PAL enzymes [25, 26-28, 46, 64-65]. All PALs recognize a short asparaginyl tripeptide tag and cleaves the peptide bond after Asn to rejoin it with the amino terminal residue of another peptide.

So far, almost all reported applications of PALs are based on P1-Asn ligations, barring a few exceptions such as the cyclization of P1-Asp peptide precursors catalyzed by the bifunctional MCoAEP2 and AtLEGβ[66, 61]. Because the acidic pH required for Asp-ligation limits the availability of the amine nucleophile, the efficiency of Asp-mediated cyclization reactions is generally much lower than that of Asn-mediated cyclization which can be performed at higher pH. There has been no reported work on the entropically more difficult intermolecular ligation at aspartyl bonds. Herein, it was found that the earlier discovered PALs, such as VyPAL2, butelase-1 and OaAEP1b, can all catalyze peptide cyclization and intermolecular ligation at aspartyl peptide bonds at acidic pH. Although PAL-mediated Asp-ligation is much less efficient than Asn-ligation, it is still faster than the ligation reactions catalyzed by sortase A by at least two orders of magnitude. The practical value of Asp-ligation in protein engineering through backbone cyclization of sfGFP and the C-terminal labeling of an affibody protein was shown herein. More importantly, the stability of a newly formed Asp-Xaa bond towards the PALs at neutral to slightly basic pH also makes it possible to conduct a second ligation reaction on the same protein at an asparaginyl junction. This pH-controlled, PAL-catalyzed tandem ligation strategy allows protein dual labeling in either N-to-C or C-to-N direction and the reactions at the two steps can be done by using the same PAL or two different PALs.

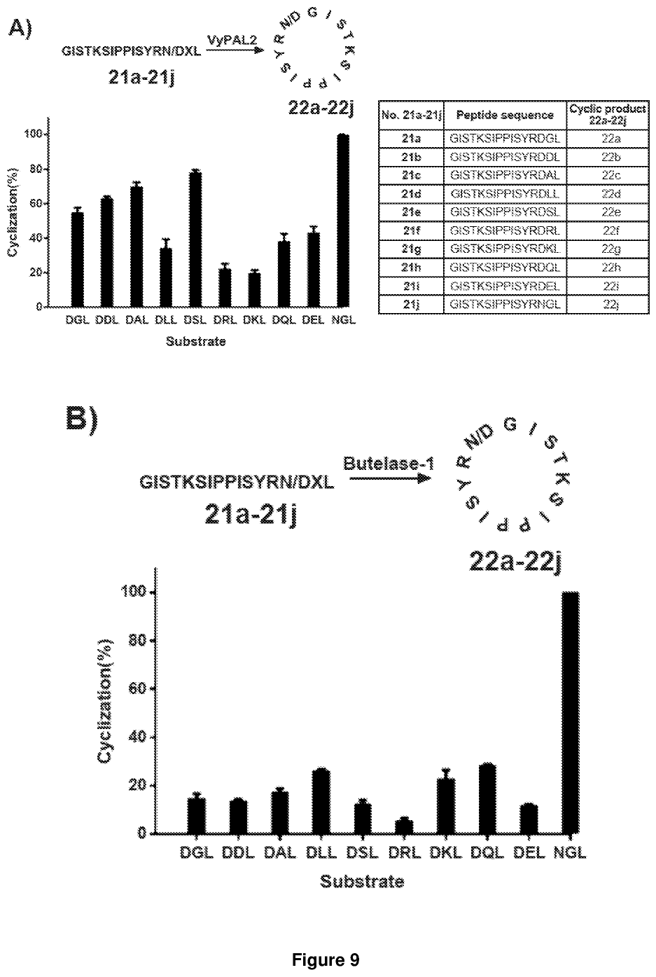

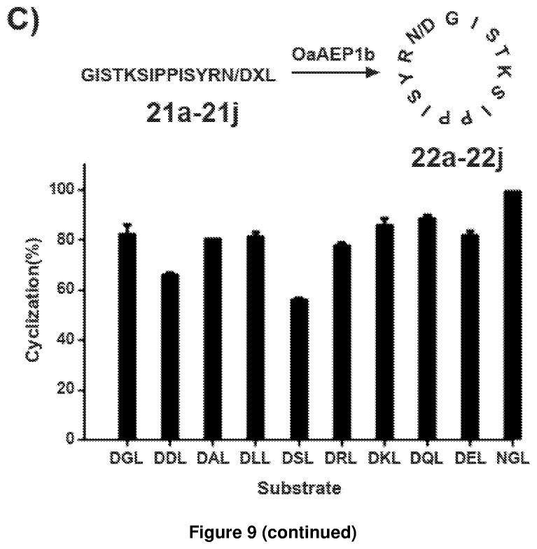

PALs recognize a tripeptide motif Asx-P1′-P2′ in the acyl donor substrate for their catalyzed ligation reactions. Because the formation of the acyl-enzyme thioester intermediate is the rate-limiting step, the leaving group P1′-P2′ also plays an important role in determining the catalytic kinetics of a particular 40 PAL-mediated reaction [14, 25, 42, 61]. Previous studies have determined Leu as a preferred P2′ residue. To identify the most favorable P1′ residues for PAL-catalyzed Asp-ligation, a panel of peptides (peptide 21a-21j) with C-terminal DXL and N-terminal GI motifs was synthesized and cyclization reactions performed. Peptide 21j with a C-terminal NGL, a P1-Asn peptide favored by VyPAL2, was used for comparison with the P1-Asp peptides 21a-21i. It was found that peptide 21e with a C-terminal DSL gave the fastest cyclization rate. These results are consistent with the previous finding that serine is one of the favored residues of VyPAL2 at the P1′ position [42]. Meanwhile, the P1′ substrate specificities of the two other ligases - Butelase-1 and OaAEP1b - were also evaluated by using the same substrates 21a-21j. It was found that butelase-1 was less efficient than VyPAL2 in catalyzing the cyclization of these P1-Asp peptides. It had a slight preference for peptide 21h-DQL, 21d-DLL and 21g-DKL over other peptides such as 21c DAL, 21a DGL, 21e DSL or 21b DDL, with 21f-DRL being the least favored P1-Asp substrate. On the other hand, OaAEP1 b exhibited good activity towards all these substrates which is mostly higher than that of VyPAL2 (FIG. 9).

It is known that the optimum pH for VyPAL2-mediated cyclization of P1-Asn substrates is near neutral [42]. To obtain a complete picture of the pH preference at both P1-Asn and P1-Asp substrates, a pH

scanning experiment using the two peptides 21e and 21j was performed. Cyclization reactions were conducted by mixing 5 μM of peptide 21e or 50 μM of 21j with 25 nM of VyPAL2 over a pH range of 4.0 to 7.4. Results confirmed that the optimum pH for the cyclization of the P1-Asn peptide 21j was 6.5-7.4. But for the P1-Asp peptide 21e (which has the C-terminal DSL tripeptide), the optimum pH was 4.0-4.5. These data corroborated findings from structural studies that, when binding to AEPs, the P1-Asp sidechain COOH is in the protonated form and its hydroxyl acts as a hydrogen bond donor. This data is also in agreement with the previous reports [60, 61, 67] that, with a pKa at ˜4.0, the P1-aspartate is protonated within the microenvironment of the enzyme's S1 pocket at pH≤4.5. As also shown in the previous studies [60, 67], because of its sensitivity to pH, this particular hydrogen bond shows different characteristics from the other three H-bonds formed with the positive hemisphere of the S1 pocket.

Homology analysis showed that the residues contributing to hydrogen bond formation were highly conserved among AEPs and PALs. Therefore, other PALs (such as butelase-1 and OaAEP1b) might also exhibit similar pH-dependent catalytic characteristics towards the P1-Asn and P1-Asp substrates.

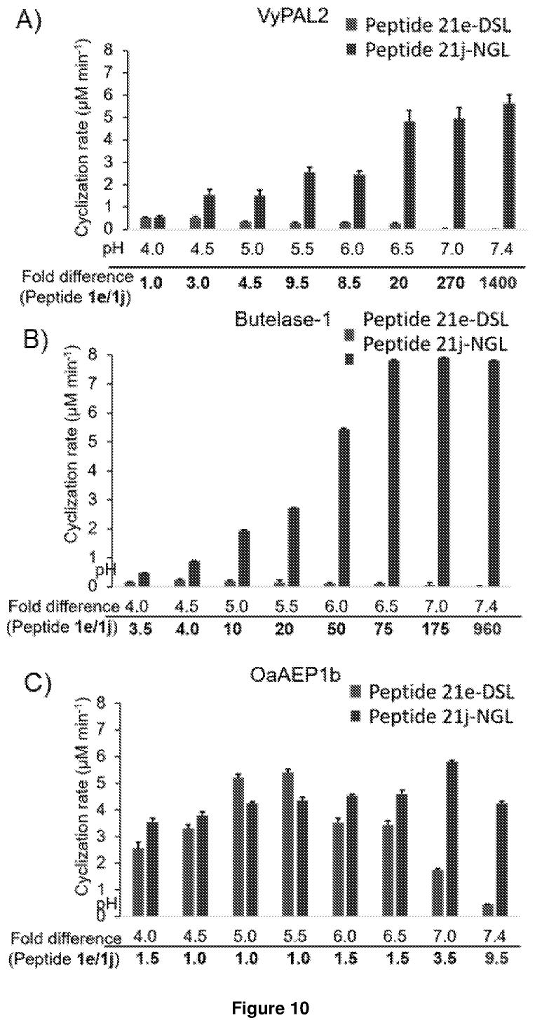

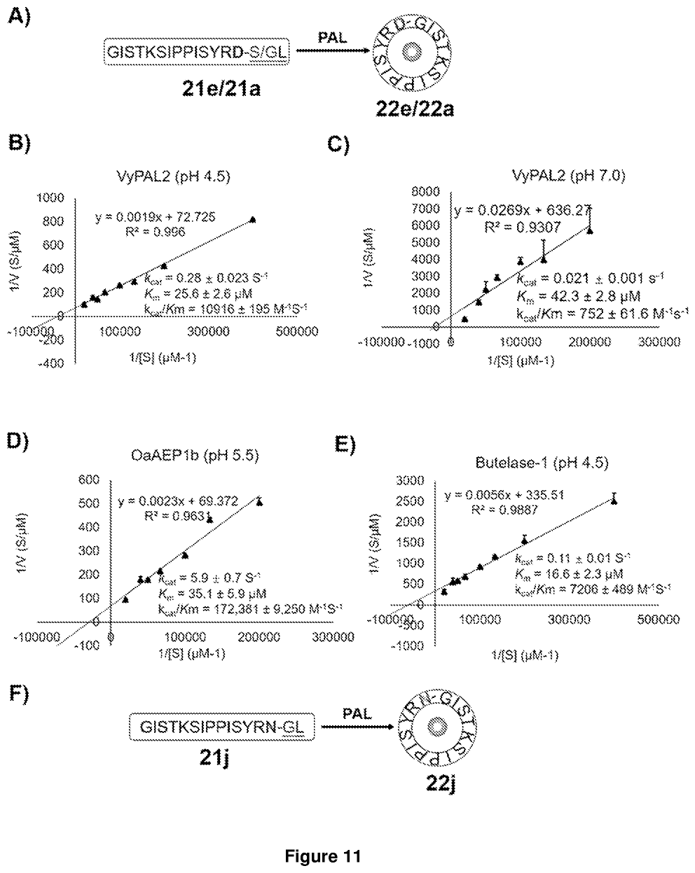

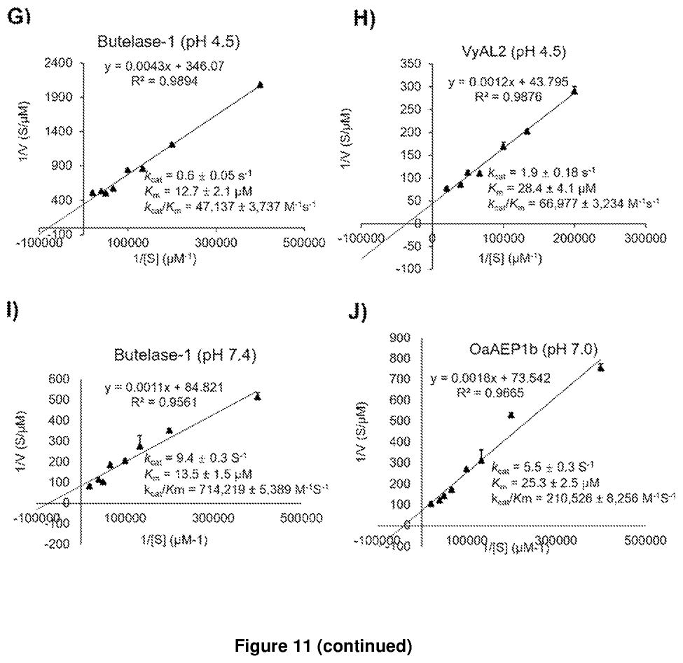

It was also found that, at the pH 7.4, the P1-Asp peptide 21e and P1-Asn peptide 21j had the largest difference in their reactivity towards VyPAL2 (with Vmax of 0.004 and 5.633 μM/min respectively). However, since the reaction rates of 21e at pH 7.4 was too slow to be determined accurately at concentrations lower than that producing Vmax, k_{cat}/Kₘ at this pH could not be determined. Therefore, catalytic kinetics of VyPAL2 on 21e at pH 7.0 were measured instead (k_{cat}/Kₘ as ˜847±123 M -1 5 1) (FIG. 10, FIG. 11 and Table 2). On the contrary, the enzymatic activity of VyPAL2 in cyclizing peptide 21j at pH 7.4 was very high with k_{cat}/Kₘ at 214445±3898 M⁻¹S⁻¹ (Table 2, FIG. 11). It is worth noting that, at pH 7.4, VyPAL2 processed peptide 21j for cyclization at a rate that is >1000 folds faster than processing peptide 21e (FIG. 10). This enormous difference of reactivity essentially established the orthogonality for ligation at Asn in the presence of aspartyl bonds.

TABLE 2
Kinetics of cyclization reactions of a P1-Asp substrates or P1-Asn substrate
catalyzed by VyPAL2, butelase-1 or OaAEP1 at acidic or near neutral pH.

| Enzyme | Substrate | pH | k_{cat} [s⁻¹] | Kₘ [μM] | k_{cat}/Kₘ [M⁻¹s⁻¹] |
|---|---|---|---|---|---|
| VyPAL2 | 1e-DSL | 4.5 | 0.28 ± 0.023 | 25.6 ± 2.6 | 10,916 ± 195 |
| | 1j-NGL | 4.5 | 1.9 ± 0.18 | 28.4 ± 4.1 | 66,977 ± 3,234 |
| | 1e-DSL | 7.0 | 0.021 ± 0.001 | 42.3 ± 2.8 | 752 ± 62 |
| | 1j-NGL | 7.4 | 5.5 ± 0.4 | 25.8 ± 1.7 | 214,445 ± 3,898 |
| Butelase- | 1e-DSL | 4.5 | 0.11 ± 0.01 | 16.6 ± 2.3 | 7,206 ± 489 |
| 1 | 1j-NGL | 4.5 | 0.6 ± 0.05 | 12.7 ± 2.1 | 47,137 ± 3,737 |
| | 1e-DSL | 7.0 | ND | ND | ND |
| | 1j-NGL | 7.4 | 9.4 ± 0.3 | 13.5 ± 1.5 | 714,219 ± 5,389 |
| OaAEP1b | 1a-DGL | 5.5 | 5.9 ± 0.7 | 35.1 ± 5.9 | 172,381 ± 9,250 |
| | 1j-NGL | 7.0 | 5.5 ± 0.3 | 25.3 ± 2.5 | 210,526 ± 8,256 |

A pH scan experiment with the other two ligases: butelase-1 and OaAEP1 b, for their cyclase activity on 21e and 21j was also conducted. As seen from FIG. 10 and Table 2, butelase-1 had a preference for the P1-Asn peptide 21j to the P1-Asp peptide 21e over the entire pH range tested and the preference was especially strong at weakly acidic and near neutral pH. In fact, the difference in reaction efficiency between 21j and 21e was about 950 times at pH 7.4 (FIG. 10). In general, butelase-1 was more active than VyPAL2 against the P1-Asn peptide 21j but less active than VyPAL2 against the P1-Asp peptide 21e. This makes VyPAL2 a more efficient ligase than butelase-1 for Asp-mediated protein modification.

Interestingly, OaAEP1 b exhibited a catalytic activity that was significantly higher than that of VyPAL2 or butelse-1 towards the P1-Asp peptide 21e at all pH, and this activity exceeded that towards 21j at pH 5.0-5.5 (FIG. 10). Surprisingly, OaAEP1b possessed substantial catalytic activity towards 21e even at pH 7 where butelase-1 or VyPAL2 was almost completely inactive (FIG. 10). It seems that the microenvironment within the 51 pocket of OaAEP1 b has a unique ability to maintain the γCOOH of P1-Asp in the protonated form. However, for OaAEP1b-catalyzed cyclization, the difference in reactivity between 21j and 21e at pH 4.0-7.4 was not very high. The biggest difference was 9.5 folds at pH 7.4. This reduced orthogonality suggests that it is not ideal to use OaAEP1b for both Asp and Asn ligations in a dual ligation scheme since the aspartyl peptide bond formed at the first step would be affected when performing the second-step ligation at the Asn junction. Nevertheless, a conceivable way of conducting pH-control orthogonal ligation on a protein would be to use OaAEP1b for Asp-ligation first, followed by ligation at the Asn using VyPAL2 or butelase-1. As discussed above, VyPAL2 is the most ideal PAL enzyme to perform single-enzyme catalyzed tandem ligation by manipulating the reaction pH.

### Example 8

### Protein Modification Using Asp-Specific Ligation at Acidic pH

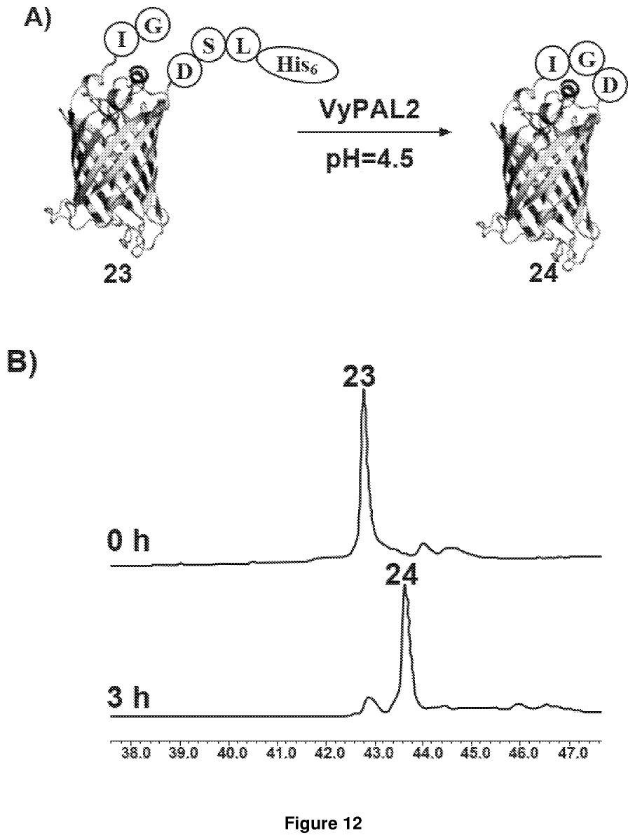

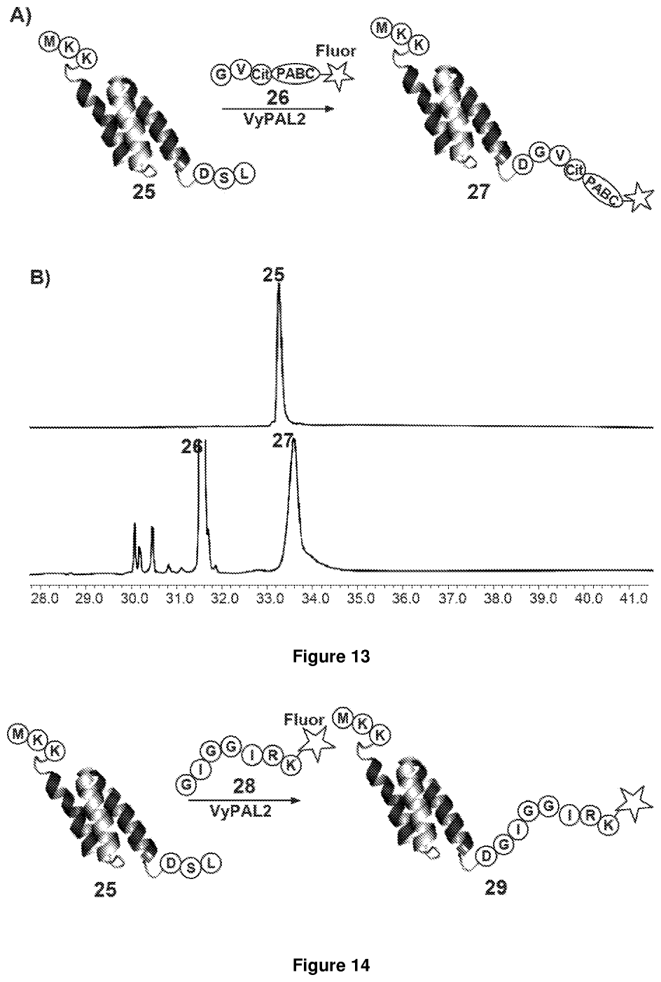

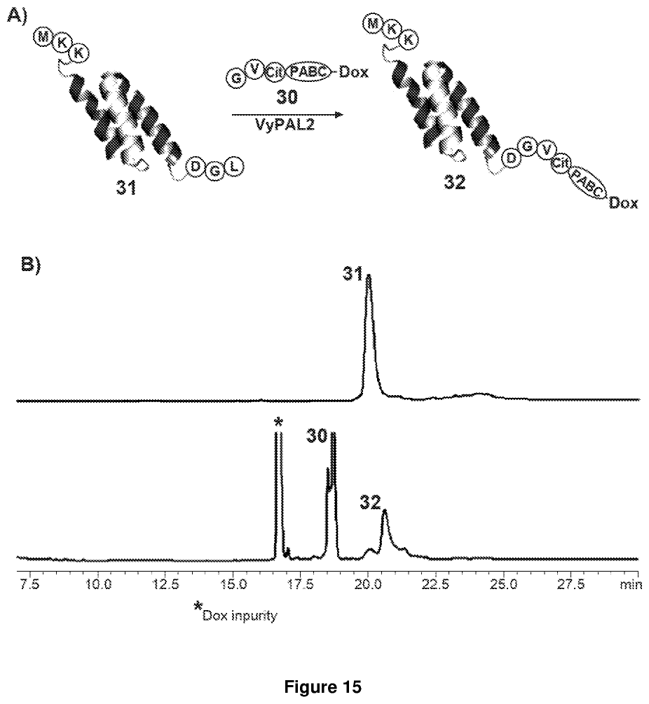

As demonstrated above, VyPAL2 is capable of mediating peptide cyclization by recognizing the DSL tripeptide motif. The results suggested the possibility of cyclizing or labeling proteins at the Asp residue. First Asp-mediated protein cyclization using sfGFP as a model was demonstrated. sfGFP-DSL-His₆ 23 (50 μM), which contains an N-terminal GI dipeptide, was mixed with 500 nM VyPAL2 at 37° C., pH 4.5. HPLC monitoring showed that, at 3 h, a yield of >80% of the cyclized product was formed, which was characterized by ESI-MS (FIG. 12). Then, protein C-terminal labelling through Asp-mediated ligation was demonstrated. Z_{EGFR-DSL} 25 (50 μM) was mixed with 250 μM fluorescein-peptide 26 or 28, followed by addition of 250 nM VyPAL2. The reaction was performed at 37° C., pH 4.5 for 4 h. As expected, an estimated >90% of labeling product was generated as analyzed by HPLC and ESI-MS (FIGS. 13 and 14). Finally, the ligation between ZEGFR and doxorubicin which was pre-functionalized with an acceptor peptide and a releasable linker, 30, to load the anti-cancer compound onto the affibody was performed. The Dox-peptide 30 was prepared by standard solution synthesis using a Boc protection group. Ligation was done by mixing ZEGFR-DGL 31 (100 μM) with 500 μM of Dox-peptide 30 and 500 nM VyPAL2 at 37° C. and pH 4.5 for 4 h. The reaction yielded >80% of the product as determined by HPLC and ESI-MS analysis (FIG. 15). At the observed rate, VyPAL2-catalyzed ligation at Asp is >2 orders of magnitude faster than sortase A ligation which requires near equal-molar quantities of the ligase. This suggests that Asp-ligation by PALs such as VyPAL2 is a practical method for protein modification.

### Example 9

### pH-Controlled Sequential Asp- and Asn-Ligations for Protein Dual Labelling

The fact that Asp-ligation can proceed at acceptable rates and the differential behaviors of the P1-Asp and Asn acyl donor substrates make it possible to conduct Asp-ligation and Asn-ligation sequentially on the same protein. This method was first tested for protein N-to-C tandem ligation. The Super Folded Green Fluorescent Protein (sfGFP) is a useful tool for biological research [68]. To apply the described method for tandem ligation at N- and -C termini of sfGFP, a truncated version of this protein was made to avoid the self-cyclization reaction, as the cyclization could be driven by spatial proximity between the two terminal ends [31, 35]. Previous study has suggested the maximum number of amino acid residues that could be truncated from its terminal ends without losing the fluorescent intensity [69]. To prepare

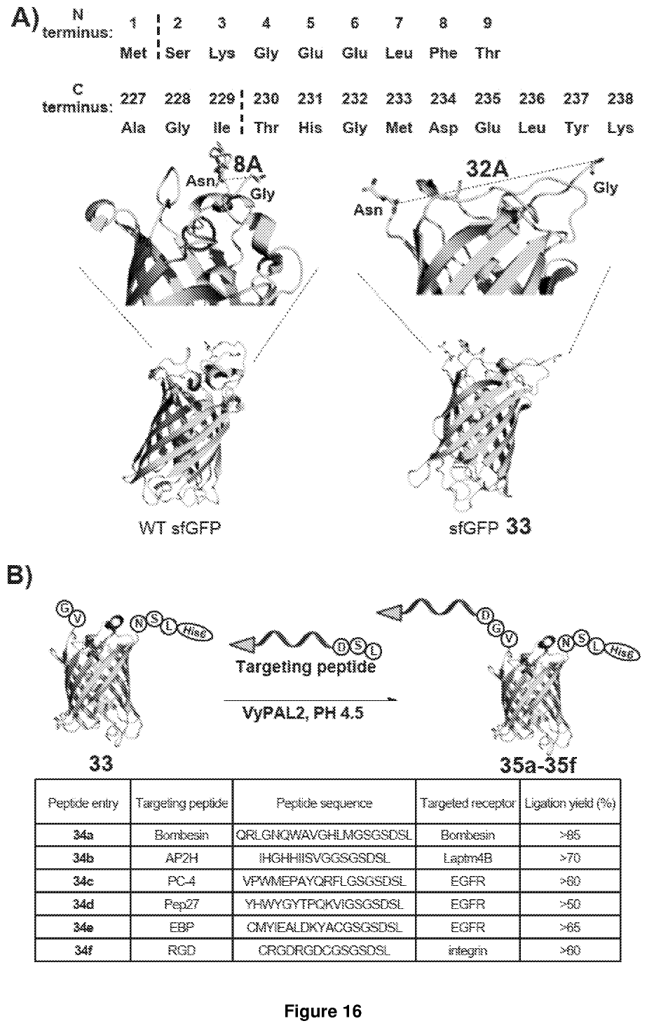

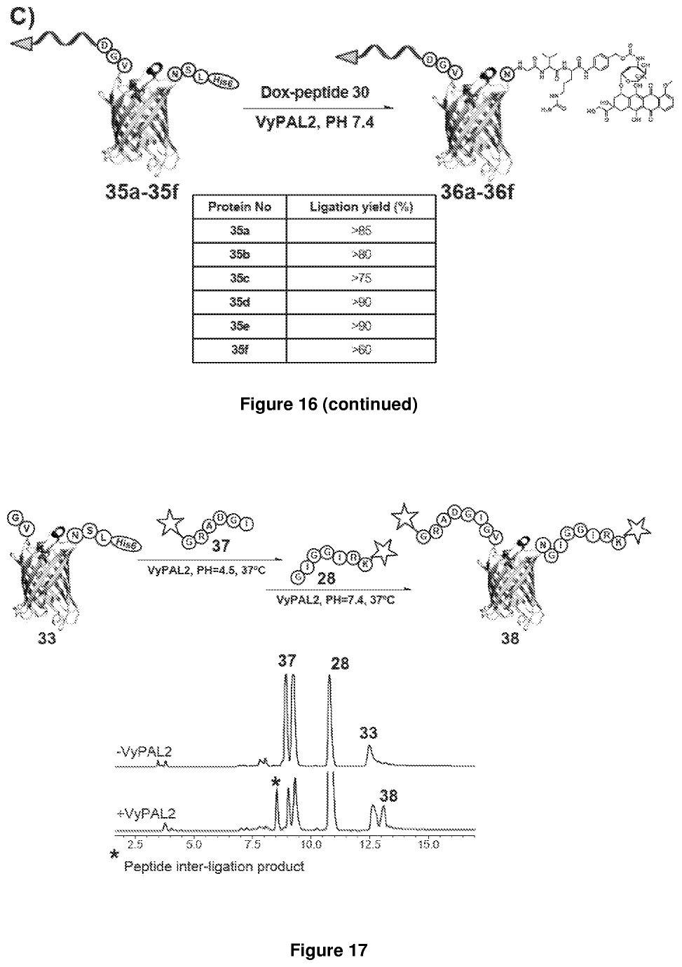

the protein suitable for ligation at both termini, a GV dipeptide and NGL tripeptide were recombinantly tagged at N- and C-terminus, respectively. Thus, a truncated formed of sfGFP 33 (2-229) with 11 residues removed from C-terminus (FIG. 16) was prepared. The distance between the N and C termini of sfGFP 33 (2-229) was much larger compared with that of the wild type sfGFP, which is 32 Å vs 8 Å (FIG. 16).

The truncated sfGFP 33 was expressed as a soluble protein without a decrease in fluorescence intensity. A list of cancer targeting peptides 34a-34f [70-74]were prepared for use in the ligation reaction with sfGFP 33. Each peptide (500 μM) was reacted with 50 μM of sfGFP 33 in the presence of 250 nM of VyPAL2 at pH 4.5 for 4 h. All the reactions afforded the products in moderate to good yields (>50%) (FIG. 16). After purification and lyophilization, the products from the 1^{st} ligation step were used for the next step reaction. In the second step, each of sfGFP 35a-35f (100 μM) was mixed with 500 μM Dox-peptide 30 and 100 nM VyPAL2 at 37° C., pH 7.4 for 1 h. All the reactions gave excellent yields (>75%), without affecting the newly formed D-GV at the N-terminus (FIG. 16C). The method was also applied for one-pot tandem ligation using VyPAL2. Peptide 37 (500 μM) was added to 50 μM of sfGFP 33 and 250 nM VyPAL2 at pH 4.5, 37° C. for 4 h. Then peptide 28 was added to the reaction mixture to a final concentration of 1 mM and the reaction was immediately adjusted to pH 7.4. After 1 h, the reaction was subjected to HPLC and ESI-MS analysis (FIG. 17). It was found that product 38 was formed at 45% from the one-pot reaction. A side reaction was observed in which peptide 37 and 28 ligated together, creating an inter-peptide ligation product (FIG. 17). Nevertheless, it was relatively easy to perform the one-pot tandem ligation scheme as the pH just needed to be adjusted after adding the second peptide, which may be very useful in large-scale preparations of dually labelled protein conjugates. Therefore, the pH-dependent activity of VyPAL2 towards P1-Asp and Asn for N- and C-terminal ligation of sfGFP was successfully utilized. Complete orthogonality was achieved at the two ligation steps. The overall N-to-C ligation scheme was established and demonstrated with excellent yields. The products 36a-36f may be useful pharmacological tools and have potential therapeutic value in the future.

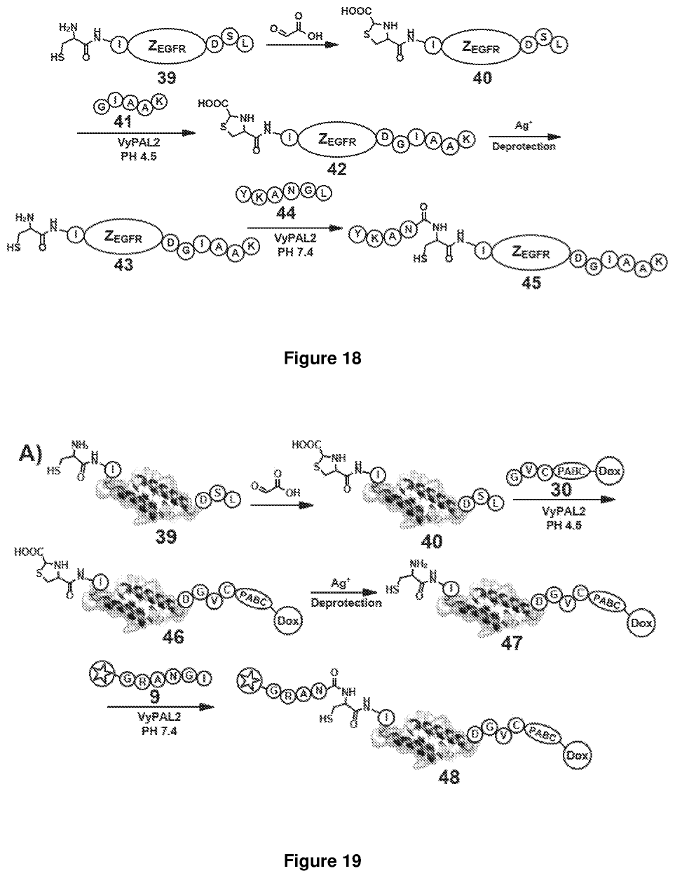

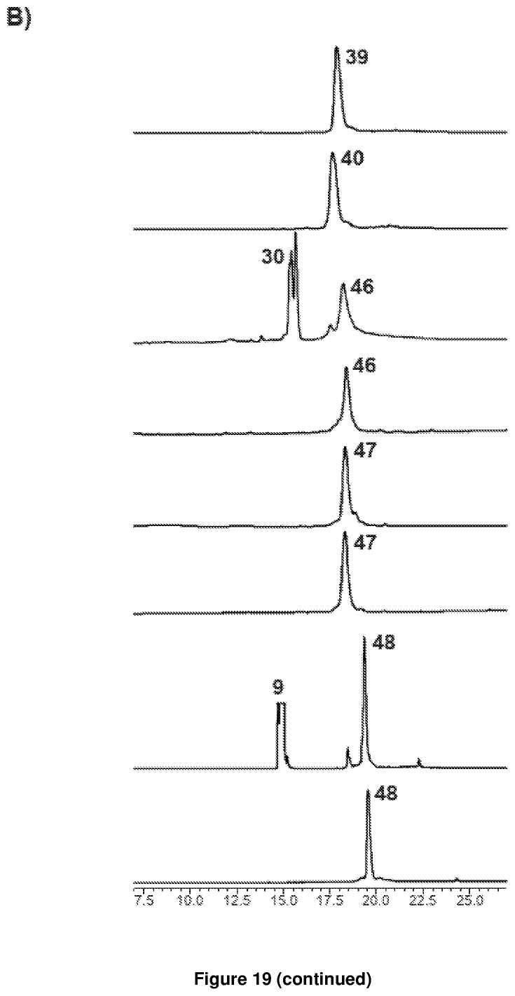

Then it was investigated whether the pH-controlled tandem ligation method could be used in the C-to-N direction. The affibody was used as a model protein for modification with functional molecules at both terminal ends. Dual labelling of the EGFR-targeting affibody with an imaging and a toxic compound would be very useful for both diagnostic and therapeutic purposes. To this end, an affibody with an N-ter “CI-” and C-ter “DSL” was prepared. Due to the thiazolidine capping formed by the N-ter cysteine residue with glyoxylic acid, affibody 40 is unable to undergo cyclization. For C-terminal labelling, affibody 40 (50 μM) was mixed with 250 μM “GI/GV-” nucleophile peptide 41 or 30 and 500 nM VyPAL2 at pH 4.5 and 37° C., resulting in the formation of 42 or 46 in 70% yield in 4 h. After purification, the thiazolidine at the N terminus of 42 or 46 was deprotected using Ag+ for 60 min to afford 43 or 47 in >95% yield. Finally, for N-terminal labelling, 250 μM of the P1-Asn peptide 44 or 9 was mixed with 43 or 47, followed by the addition of 50 nM VyPAL2 and adjusting the pH to 7.4. After 30 min, about 85% of product 45 or 48 was formed (FIGS. 18 and 19). It is worth noting that, Val-Cit-PABC, designed for the releasing of Dox, is a well-established cathepsin B-sensitive linker [75]. Therefore, the results show this

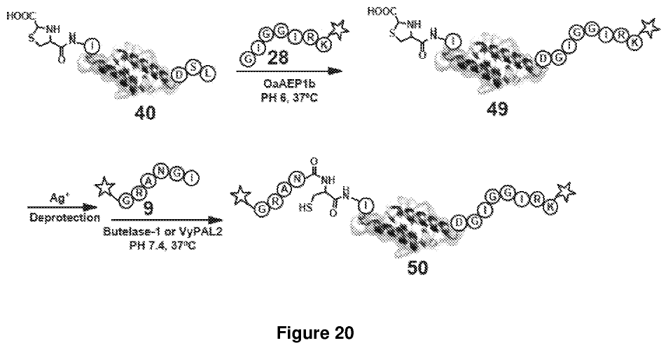

pH-controlled orthogonal tandem ligation scheme can also be conducted in the C-to-N direction. Meanwhile, sequential ligation utilizing two PALs was also evaluated. As shown before, OaAEP1b is quite active towards P1-Asp substrates at pH ranging from 4.0-7.4, whereas butelase-1 and VyPAL2 show optimum activity to P1-Asp substrates only at acidic pH and to P1-Asn substrates at around neutral pH (FIG. 10, Table 2). Therefore, a tandem ligation method using OaAEP1b for Asp-ligation and butelase-1 or VyPAL2 for Asn-ligation was employed. Two fluorescein-peptides at the affibody's N- and -C terminus, respectively, were successfully ligated using the OaAEP1b/butlease-1 or OaAEP1 b/VyPAL2 ligase pair. Both pairs worked with high efficiency and specificity, leading to the formation of protein 50, a dual fluorescein labeled affibody (FIG. 20). In summary, the usefulness of PAL enzymes as pH-controllable biocatalysts capable of performing sequential ligation at aspartyl and asparaginyl junctions was demonstrated.

### Example 10

### Bioimaging and Cytotoxicity of the Dual-Labelled Affibody Prepared Using the pH-Controlled Tandem Ligation Method

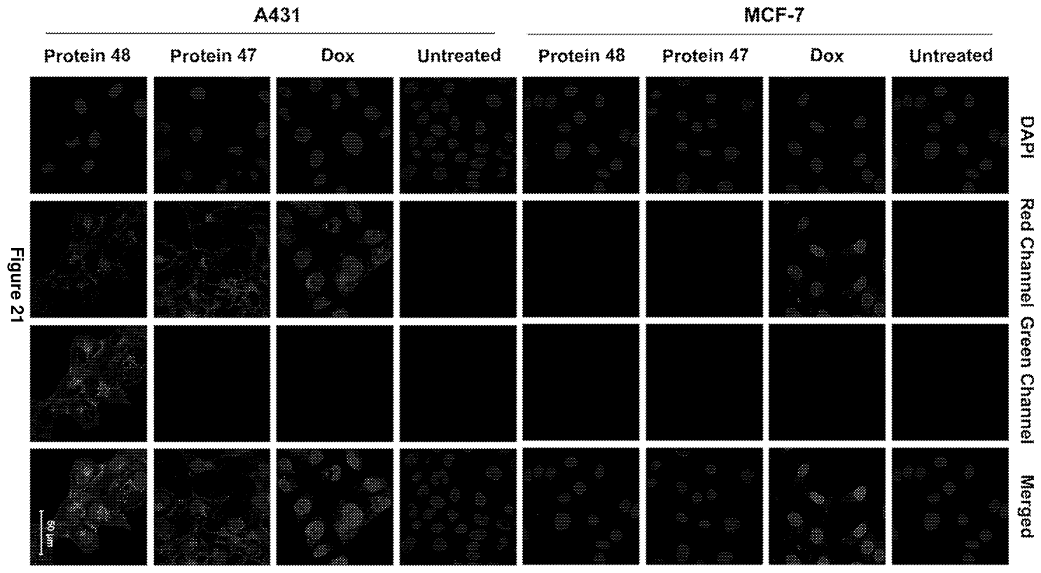

Next, confocal microscopy analysis was performed to determine the binding and inhibitory effects of the protein conjugate 48 on MCF-7 and A431 cell lines. The intrinsic fluorescence of doxorubicin and fluorescein serves as an imaging tool to visualize the binding of 48 to the cells. The overlapping of red fluorescent doxorubicin and green fluorescent fluorescein gave a color of yellow (FIG. 21). As shown in the figures, only the EGFR-overexpressing A431 cells were positively stained after a 30-min treatment with 47 and 48. The same treatment did not yield any staining on the EGFR-negative MCF-7 cells. On the other hand, both cell lines were stained by the free doxorubicin, which is not surprising as it can enter cells and bind to nuclear DNA.

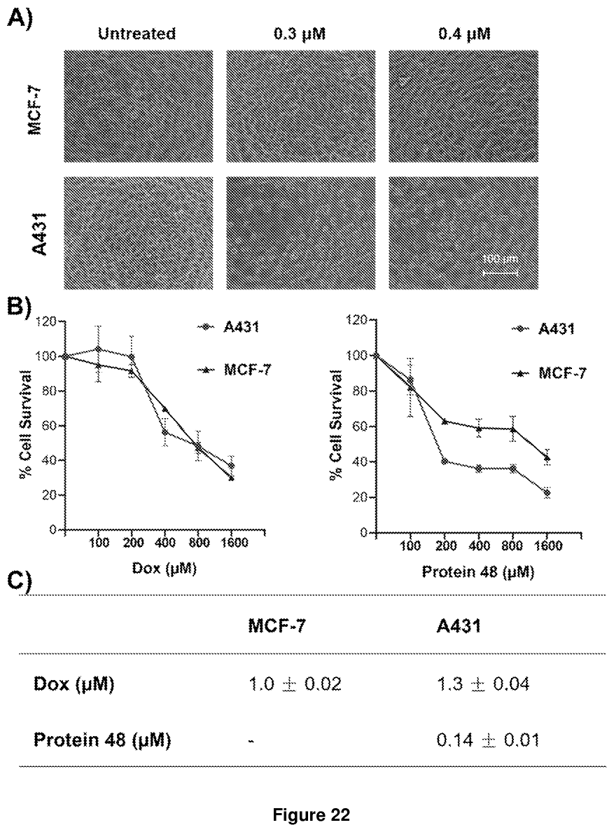

Bright Field Microscopy analysis and MTT assay were performed to determine the inhibitory effects of 48 on MCF-7 and A431 cell lines. In the experiments, both cell lines were treated with 0.3 and 0.4 μM protein 48 for 36 h and subjected to microscopy analysis. At this concentration, 48 exhibited substantial cytotoxic effect on A431 cells, with smaller effects observed in the other control settings (FIG. 22A). Next, MTT assay was performed to determine the IC₅₀. Dox and protein 48 were added at varying concentrations to MCF-7 and A431 cells for 36 h. As shown in FIG. 22B, 48 showed significantly higher toxicity with an IC₅₀ of 0.14 ±0.01 μM to A431 cells than to MCF-7 as IC₅₀ with this cell line could

not be determined which is consistent to previously published results [58]. The unconjugated Dox exhibited relatively low cytotoxicity to A431 cells compared to 48, likely due to a lack of enrichment of the compound in the cells. The measured IC₅₀ of Dox in MCF-7 and A431 was 1.0±0.02 μM and 1.3±0.04 μM, respectively (FIG. 22C). The enhanced toxicity of Dox conjugate 48 might be due to the fast enrichment of the conjugate via receptor-mediated endocytosis which uptook the conjugate through the endosomal pathway and delivered it to the lysosome. Subsequently, the cathepsin B protease in this organelle cleaved the Val-Cit-PABC linker to release Dox. Attributing to its hydrophobic property, Dox could escape easily from lysosomes and finally get enriched in the nuclei to bind to DNA, leading to cell apoptosis [76].

The examples provided demonstrate the feasibility to exploit the different substrate specificity of butelase-1 and VyPAL2 to develop a new tandem Asn-ligation method for bio-orthogonal dual modification of proteins under mild aqueous conditions at near neutral pH. This novel bio-orthogonal method has been used to prepare a dual-labeled affibody as a selective imaging and cytotoxic agent for breast cancer cells. It could be shown that the bio-orthogonal ligation scheme is bi-directional, as it can be executed in both N-to-C and C-to-N directions enabling the synthesis of the affibody conjugate 12. Furthermore, the scheme was extended to the preparation of a cyclic affibody conjugated with the cytotoxic compound doxorubicin. Unlike the hydrophobic free doxorubicin which is poorly soluble in water, the prepared cycloaffibody-DOX conjugate 20 has excellent water solubility. Such a conjugate is also expected to have lower cardiotoxicity than free doxorubicin. A backbone-cyclized protein is known to have increased thermal, chemical and proteolytic stability. The data prove that the prepared linear and cyclic affibody conjugates 12 and 20 showed uncompromised high binding affinity and enhanced cytotoxicity toward EGFR-overexpressing A431 cells.

It has also been shown that PALs exhibit substantial ligation activity towards P1-Asp substrates at acidic pH and exploited the influence of pH on the specificity and activity of these ligases towards P1-Asn/Asp substrates to develop a new method for protein sequential ligation. This method has been used to prepare a dual-labelled sfGFP and affibody as a selective imaging and cytotoxic agent for cancer cells. The ligation scheme can be executed both from the N-to-C and C-to-N directions, enabling the synthesis of dually labelled sfGFP and affibody conjugates. The prepared affibody conjugates 48 showed uncompromised high binding affinity and enhanced cytotoxicity toward EGFR-overexpressing A431 cells.

These findings point to the promise of PALs as precision biomanufacturing tools for complex bioconjugates with multiple functionalities and unusual structures. One can also envisage the use of these ligases for the functionization of protein nanoparticles. Therefore, the methodologies described herein may pave a new way to the development of next-generation protein-based theranostics for the diagnosis, prevention and treatment of human diseases.

Abbreviations PALs, peptidyl asparaginyl ligases; POI, protein of interest; AEP, Asparaginyl Endopeptidase; BML, butelase-mediated ligation; VML, VyPAL-mediated ligation; NHV, Asn-His-Val tripeptide; NGF, Asn-Gly-Phe tripeptide; Vy, Viola yedoensis; PBS, phosphate saline buffer; Ni-NTA, nitrilotriacetic acid-nickel; DMEM, Dulbecco's Modified Eagle Medium; FBS, fetal bovine serum; EDTA, Ethylenediaminetetraacetic acid; MTT, 3-(4,5-Dimethylthiazol-2-yl)-2,5-Diphenyltetrazolium Bromide; DAPI, 4′, 6-Diamidino-2-Phenylindole, Dihydrochloride; MBHA, 4-Methylbenzhydrylamine; Fmoc, Fluorenylmethyloxycarbonyl; Boc, tert-butyloxycarbonyl; TFA, Trifluoroacetic acid; HPLC, High-performance liquid chromatography; TIS, Triisopropylsilane; ESI-MS, electrospray ionization mass spectrometry; KD, equilibrium dissociation constant; DMF, dimethylformamide; DCM, Dichloromethane; PyBOP, benzotriazol-1-yl-oxytripyrrolidinophosphonium hexafluorophosphate; DI PEA, N,N-Diisopropylethylamine; cPDC, cycloprotein-drug conjugate; IPTG, Isopropyl β-D-1-thiogalactopyranoside; EGFR, epidermal growth factor receptor; K_{D}, dissociation constant; DOX, doxorubicin; SPPS, solid phase peptide synthesis

### REFERENCES

1. Keikar SS, Reineke TM. Theranostics: combining imaging and therapy. Bioconjug. Chem. 2011; 22:1879-1903.

2. Funkhouser, J. Reintroducing pharma: Theranostic revolution. Curr. Drug Discovery 2002; 2: 17-19.

3. Xie J, Chen X. Nanoparticle-based theranostic agents. Adv. Drug Deliv. Rev. 2010; 62: 1064-1079.

4. Luo S, Yang X, Shi C. Newly emerging theranostic agents for simultaneous cancer-targeted imaging and therapy. Curr. Med Chem. 2016; 23: 483-497.

5. Langbein T, Weber WA, Eiber M. Future of theranosctics: An outlook on precision oncology in nuclear medicine. J. Nucl. Med. 2019; 60: 13S-19S.

6. Sumer B, Gao JM. Theranostic nanomedicine for cancer, Nanomedicine 2008; 3: 137-140.

7. Chen H, Zhang W, Zhu G, Xie J, Chen X. Rethinking cancer nanotheranostics. Nat. Rev. Mater, 2017; 2: 17024,

8. Lim EK, Kim T, Paik 5, Haam S, Huh YM, Lee K. Nanomaterials for theranostics: recent advances and future challenges. Chem Rev, 2015;115(1): 327-394,

9. Zhang L, Jing D, Jiang N, Rojalin T, et al. Transformable peptide nanoparticles arrest HER2 signalling and cause cancer cell death in vivo. Nature Nanotech. 2020; 15: 145-153.

10. Dammes N, Peer D. Monoclonal antibody-based molecular imaging strategies and theranostic opportunities, Theranostics 2020; 10: 938-955.

11. Moek KL, Giesen D, Kok IC, et al. Theranostics using antibodies and antibody-related therapeutics. J Nucl Med. 2017; 58: 83S-90S,

12. Hoyt EA, Cal PMSD, Oliverira BL, Bernardes GJ. Contemporary approaches to site-selective protein modification. Nat. Rev. Chem. 2019; 3: 147-171.

13. Spicer CD, Davis BG, Selective chemical protein modification. Nat. Commun. 2014; 5: 4740.

14. Lotze J, Reinhardt U, Seitz O, Beck-Sickinger G. Peptide-tags for site-specific protein labelling in vitro and in vivo. Mol. Biosyst. 2016; 12: 1731-1745.

15. Abrahmsen L, Tom J, Burnier J, Butcher KA, Kossiakoff A, Wells JA. Engineering subtilisin and its substrates for efficient ligation of peptide bonds in aqueous solution. Biochem. 1991; 30: 4151-4159.

16. Chang K, Jackson DY, Burnier JP, Wells JA. Subtiligase: a tool for semisynthesis of proteins. Proc. Natl. Acad. Sci. 1994; 91: 12544-12548.

17. Henager SH, Chu N, Chen Z, Bolduc D, Dempsey DR, Hwang Y, Wells J, Cole PA. Enzyme-catalyzed expressed protein ligation. Nat. Methods. 2016; 13: 925-927.

18. Tan X, Yang R, Liu C-F. Facilitating subtiligase-catalyzed peptide ligation reactions by using peptide thioester substrates. Org. Lett. 2018; 20: 6691-6694.

19. Weeks M, Wells JA. Subtiligase-catalyzed peptide ligation. Chem. Rev. 2020; 120: 3127-3160.

20. Schneewind O, Fowler A, Faull KF. Structure of the cell wall anchor of surface proteins in Staphylococcus aureus. Science 1995; 268: 103-106.

21. Mao H, Hart SA, Schink A, Pollok BA. Sortase-mediated protein ligation: a new method for protein engineering. J. Am. Chem. Soc. 2004; 126: 2670-2671.

22. Popp MW, Antos JM, Grotenbreg GM, Spooner E, Ploegh HL. Sortagging: a versatile method for protein labeling. Nat. Chem. Biol. 2007; 3: 707-708.

23. Williamson DJ, Fascione M. A, Webb ME, Turnbull WB. Efficient N-terminal labeling of proteins by use of sortase. Angew. Chem. Int. Ed. 2012; 51: 9377-9380.

24. Pishesha N, Ingram JR, Ploegh HL. Sortase A: a model for transpeptidation and its biological applications. Annu. Rev. Cell. Dev. Biol. 2018; 34: 163-188.

25. Nguyen GK, Wang S, Qiu Y, Hemu X, Lian Y, Tam JP. Butelase 1 is an Asx-specific ligase enabling peptide macrocyclization and synthesis. Nat. Chem. Biol. 2014; 10: 732-738.

26. Nguyen GK, Kam A, Loo S, Jansson AE, Pan LX. Tam JP. Butelase 1: a versatile ligase for peptide and protein macrocyclization. J. Am. Chem. Soc. 2015; 137: 15398-15401.

27. Nguyen GK, Qiu Y, Cao Y, Hemu X. ; Liu C-F, Tam J. P. Butelase-mediated cyclization and ligation of peptides and proteins. Nat. Protoc. 2016; 11: 1977-1988.

28. Bi X, Yin J, Nguyen GKT, Rao C, Halim NBA, Hemu X, Tam JP, Liu C-F. Enzymatic engineering of live bacterial cell surfaces using butelase 1. Angew. Chem. Int. Ed. 2017; 56: 7822-7825.

29. Bi X, Yin J, Zhang D, Zhang X, Balamkundu S, Lescar J, Dedon P, Tam JP, Liu CF. Tagging transferrin receptor with a disulfide FRET probe to gauge the redox state in endosomal compartments. Anal. Chem. 2020; 92: 12460-12466.

30. Müntz K, Shutov AD. Legumains and their functions in plants. Trends in Plant Science 2002;7: 340-344.

31. Dall, E., and Brandstetter, H. Struction and function of legumain in health and disease. Biochimie 2016; 122: 126-150.

32. Min, W., and Jones, D.H. In vitro splicing of concanavalin A is catalyzed by asparaginyl endopeptidase. Nat. Struct. Mol. Biol. 1994; 1: 502-504.

33. Gillon AD, Saska I, Jennings CV, Guarino RF, Craik DJ, Anderson MA. Biosynthesis of circular proteins in plants. Plant J. 2008; 53: 505-515.

34. James, A.M., Haywood, J., and Mylne, J. S. Unravelling the mode of action of plant proteases. New Phytologist 2018; 218: 923-928.

35. Bernath-Levin K, Nelson C, Elliott AG, Jayasena A S, Millar A. H, Craik D. J, Mylne J. S. Peptide macrocyclization by a bifunctional endoprotease. Chem. Biol. 2015; 22: 571-582.

36. Harris S, Durek T, Kaas Q, Poth AG, Gilding EK, Conlan BF, Saska I, Daly NL, Weerden NLVD, Craik DJ. Efficient backbone cyclization of linear peptides by a recombinant asparaginyl endopeptidase. Nat Commun 2015; 6: 10199.

37. Yang R, Wong YH, Nguyen GKT, Tam JP, Lescar J, Wu B. Engineering a catalytically efficient recombinant protein ligase. J Am Chem Soc 2017; 139: 5351-5358.

38. Zauner FB, Dall E, Regl C, Grassi L, Huber CG, Cabrele C, Brandstetter H. Crystal structure of plant legumain reveals a unique two-chain state with pH-dependent activity regulation. The Plant Cell 2018; 30: 686-699.

39. Jackson MA, Gilding E, Shafee T, Harris K, Kaas Q, Poon S, Yap K, Jia H, Guarino R, Chan L, Durek T, Anderson MA, Craik DJ. Molecular basis for the production of cyclic peptides by plant asparaginyl endopeptidases. Nat Commun 2018; 9: 2411.

40. Zauner FB, Elsasser B, Dall E, Cabrele C, Brandstetter H. Structural analyses of Arabidopsis thaliana legumain y reveal differential recognition and processing of proteolysis and ligation substrates. J Biol Chem. 2018; 293: 8934-8946.

41. James AM, Haywood J, Leroux J, Ignasiak K, Elliott AG, Schmidberger JW, Fisher MF, Nonis SG, Fenske R, Bond CS, Mylne JS. The macrocyclizing protease butelase 1 remains autocatalytic and reveals the structural basis for ligase activity. Plant J 2019; 98: 988-999.

42. Hemu X, Sahili AEI; Hu S, Wong K, Chen Y, Wong YH, Zhang X, Serra A,Goh BC, Darwis DA, Chen MW, Sze SK, Liu C-F, Lescar J, Tam JP. Structural determinants for peptide-bond formation by asparaginyl ligases. Proc. Natl. Acad. Sci. USA 2019; 116: 11737-11746.

43. Hemu X, To J, Zhang X, Tam JP. Immobilized Peptide Asparaginyl Ligases Enhance Stability and Facilitate Macrocyclization and Site-specific Ligation. J. Org. Chem 2019; 3: 1504-1512.

44. Ling JJ, Policarpo RL, Rabideau AE, Liao X, Pentelute B. L. Protein thioester synthesis enabled by sortase. J. Am. Chem. Soc. 2012; 134: 10749-10752.

45. Li Y-M, Li Y-T, Pan M, Kong X-Q, Huang Y-C, Hong Z-Y, Liu L. Irreversible site-specific hydrazinolysis of proteins by use of sortase. Angew. Chem. Int. Ed. 2014; 53: 2198-2202.

46. Cao Y, Nguyen GK, Tam JP, Liu C-F. Butelase-mediated synthesis of protein thioesters and its application for tandem chemoenzymatic ligation. Chem Commun 2015; 51: 17289-17292.

47. Harmand TJ, Bousbaine D, Chan A, Zhang X, Liu DR, Tam JP, Ploegh HL. One-pot dual labeling of IgG 1 and preparation of C-to-C fusion proteins through a combination of sortase A and butelase 1. Bioconjug Chem 2018; 29: 3245-3249.

48. Rehm FB, Harmand TJ, YAP K, Durek T, Craik D J, Ploegh HL. Site-specific sequential protein labeling catalyzed by a single recombinant ligase. J. Am. Chem. Soc. 2019; 141: 17388-17393.

49. Antos JM, Chew GL, Guimaraes CP, Yoder NC, Grotenberg GM, Popp MWL, Ploegh HL. Site-Specific N- and C-Terminal Labeling of a Single Polypeptide Using Sortases of Different Specificity. J. Am. Chem. Soc. 2009; 131: 10800-10801.

50. Nord K, Gunneriusson E, Ringdahl J, Stahl S, Uhlen M, Nygren P-A. Binding proteins selected from combinatorial libraries of an a-helical bacterial receptor domain. Nat. Biotechnol. 1997; 15: 772-777.

51. Friedman M, Orlova A, Johansson E, Eriksson TL, Höldén-Guthenberg I, Tolmachev V, Nilsson FY, Stahl S. Directed evolution to low nanomolar affinity of a tumor-targeting epidermal growth factor receptor-binding affibody molecule. J. Mol. Biol. 2008; 376: 1388-1402.

52. Ellerby HM, Arap W, Ellerby LM, Kain R, Andrusiak R, Del Rio G, Krajewski S, Lombrado CR, Rao R, Ruoslahti E, Bredesen DE, Pasqualini R. Anti-cancer activity of targeted pro-apoptotic peptides. Nat Medicine 1999; 5: 1032-1038.

53. Dubowchik GM, Firestone RA, Padilla L, Willner D, Hofstead SJ, Mosure K, Knipe JO, Lasch SJ, Trail PA. Cathepsin B-labile dipeptide linkers for lysosomal release of doxorubicin from internalizing immunoconjugates: model studies of enzymatic drug release and antigen-specific in vitro anticancer activity. Bioconjug Chem. 2002; 13: 855-869.

54. Davidson NE, Gelmann EP, Lippman ME, Dickson DB. Epidermal growth factor receptor gene expression in estrogen receptor-positive and negative human breast cancer cell lines. Mol. Endocrinol. 1987; 1: 216-223.

55. Nakase I, Okumura S, Katayama S, Hirose H, Pujals S, Yamaguchi H, Arakawa S, Shimizu S, Futaki S. Transformation of an antimicrobial peptide into a plasma membrane-permeable mitochondria-targeted peptide via the substitution of lysine with arginine. Chem. Commun. 2012; 48: 11097-11099.

56. Jin Y, Song L, Su Yue, Zhu L, Pang Y, Qiu F, Tong G, Yan D, Zhu B, Zhu X. Oxime linkage: a robust tool for the design of pH-sensitive polymeric drug carriers. Biomacromolecules 2011; 12: 3460-3468.

57. Dirksen A, Dawson PE. Rapid oxime and hydrazone ligations with aromatic aldehydes for biomolecular labeling. Bioconj. Chem 2008; 19: 2543-2548.

58. Lee SB, Hassan M, Fisher R, Chertow O. Chernomordik V, Kramer-Marek G, Gandjbakhche A, Capala J. Affibody molecules for in vivo characterization of HER2-positive tumors by near-infrared imaging. J. Clin. Cancer. Res. 2008; 14: 3840-3849.

59. Hemu X, Sahili AEI., Hu S., Zhang X, Serra A, Goh BC, Darwis DA, Chen MW, Sze SK, Liu C-F, Lescar J, Tam JP. Turning an Asparaginyl Endopeptidase into a Peptide Ligase. ACS Catal. 2020, 10: 8825-8834.

60. Dall E, Brandstetter H. Activation of legumain involves proteolytic and conformational events, resulting in a context- and substrate-dependent activity profile. Acta Crystallogr Sect F Struct Biol Cryst Commun, 2012, 68: 24-31.

61. Dall E, Zauner FB, Soh WT, Demir F, Dahms SO, Cebrele C, Huesgen PF, Brandstetter H. Structural and functional studies of Arabidopsis thaliana legumain beta reveal isoform specific mechanisms of activation and substrate recognition. J. Biol. Chem. 2020, 295: 13047-13064.

62. Zhao L, Hua T, Crowley C, Ru H, Ni X, Shaw N, Jiao L, Ding W, Qu L, Hung L-W, Huang W, Liu L, Ye K, Ouyang S, Cheng G, Liu ZJ. Structural analysis of asparaginyl endopeptidase reveals the activation mechanism and a reversible intermediate maturation stage. Cell Res. 2014: 24,344-358.

63. Dall E, Fegg J C, Briza P, Brandstetter H. Structure and mechanism of an aspartimide-dependent peptide ligase in human legumain. Angew. Chem. Int. Ed. 2015, 54: 2917-2921.

64. Cao Y, Nguyen GK, Chuah S, Tam JP, Liu CF. Butelase-mediated ligation as an efficient bioconjugation method for the synthesis of peptide dendrimers. Bioconjug. Chem, 2016, 27: 2592-2596.

65. Bi X, Yin J, Hemu X, Rao, C, Tam JP, Liu CF. Immobilization and intracellular delivery of circular proteins by modifying a genetically incorporated unnatural amino acid. Bioconjugate chemistry, 2018, 29: 2170-2175.

66. Du J, Yap K, Chan LY, Rehm FBH, Looi FY, Poth AG, Gilding EK, Kaas Q, Durek T, Craik DJ. A bifunctional asparaginyl endopeptidase efficiently catalyzes both cleavage and cyclization of cyclic trypsin inhibitors. Nat Commun 2020, 11: 1575.

67. Dall E, Brandstetter H. Proc. Natl. Acad. Sci. USA. 2013, 110: 10940-10945.

68. Zimmer M. Chem. Rev. Green fluorescent protein (GFP): applications, structure, and related photophysical behavior. 2002, 102, 759-782.

69. Li X, Zhang G, Ngo N, Zhao X, Kain S, Huang C-C. Deletions of the Aequorea victoria green fluorescent protein define the minimal domain required for fluorescence. J. Biol. Chem. 1997, 272: 28545-28649.

70. Xu Y, Huang W, Ren G, Qi S, Jiang H, Miao Z, Liu H, Lucente E, Bu L, Shen B, Barron A, Cheng Z. A Four-Arm Star-Shaped Poly(ethylene glycol) (StarPEG) Platform for Bombesin Peptide Delivery to Gastrin-Releasing Peptide Receptors in Prostate Cancer. ACS Macro Lett. 2012, 6: 753-757.

71. Jin Y, Huang Y, Yang H, Liu G, Zhao R. A peptide-based pH-sensitive drug delivery system for targeted ablation of cancer cells Chem. Commun. 2015, 51, 14454-14457.

72. Hossein H, Althagafi E, Kaur K. Small peptide ligands for targeting EGFR in triple negative breast cancer cells Sci. Rep. 2019, 9, 2723.

73. Ai S, Duan J, Liu X, Bock S, Tian Y, Huang Z. Biological evaluation of a novel doxorubicin-peptide conjugate for targeted delivery to EGF receptor-overexpressing tumor cells. Mol. Pharm. 2011, 2: 375-386.

74. Liu J, Cheng X, Tian X, Guan D, Ao J, Wu Z, Huang W, Le Z. Design and synthesis of novel dual-cyclic RGD peptides for αvβ3 integrin targeting. Bioorg. Med. Chem. Lett. 2019, 7: 896-900.

75. Zhong Y-J, Shao L-H, Li Y. Cathepsin B-cleavable doxorubicin prodrugs for targeted cancer therapy. Int. J. Oncol. 2013, 42: 373-383.

76. Yang F, Teves SS, Kemp CJ, Henikoff S. Doxorubicin, DNA torsion, and chromatin dynamics. Biochim Biophys Acta Rev Cancer 2014, 1845: 84-89.

All documents referred to herein are incorporated by reference in their entirety.

1. Method for (poly)peptide tandem ligation, the method comprising the steps of:
(i) contacting a first (poly)peptide (A) having at its C-terminus a binding and ligation site for an asparaginyl ligase with a second (poly)peptide (B) to be ligated to said first (poly)peptide and a first asparaginyl ligase (C) under conditions that allow ligation of the second (poly)peptide to the C- or N-terminus of the first (poly)peptide to yield a modified first (poly)peptide;
(ii) contacting the modified first (poly)peptide obtained in step (i) with a third (poly)peptide (D) to be ligated to said modified first (poly)peptide and a second asparaginyl ligase (E) under conditions that allow ligation of the third (poly)peptide to the C- or N-terminus of the first (poly)peptide to yield a dually modified first (poly)peptide;
wherein the first and second asparaginyl ligase are selected from VyPAL2 comprising or consisting of the amino acid sequence set forth in SEQ ID NO:1 and variants thereof that share at least 80% sequence identity with the amino acid sequence set forth in SEQ ID NO:1 over their entire length, and butelase-1 comprising or consisting of the amino acid sequence set forth in SEQ ID NO:2 and variants thereof that share at least 80% sequence identity with the amino acid sequence set forth in SEQ ID NO:2 over their entire length.

2. The method of claim 1, wherein the second (poly)peptide has at its C-terminus a binding and ligation site for the first asparaginyl ligase and is ligated to the N-terminus of the first (poly)peptide by the first asparaginyl ligase.

3. The method of claim 2, wherein the binding and ligation site for an asparaginyl ligase at the C-terminus of the first (poly)peptide is for the second asparaginyl ligase, and wherein the third (poly)peptide is ligated to the C-terminus of the first (poly)peptide by the second asparaginyl ligase.

4. (canceled)

5. The method of claim 1, wherein the second (poly)peptide is ligated to the C-terminus of the first (poly)peptide by the first asparaginyl ligase, wherein the binding and ligation site for an asparaginyl ligase at the C-terminus of the first (poly)peptide is for the first asparaginyl ligase, and wherein the third (poly)peptide has at its C-terminus a binding and ligation site for the second asparaginyl ligase and is ligated to the N-terminus of the first (poly)peptide by the second asparaginyl ligase.

6. (canceled)

7. The method of claim 1, wherein the first and second asparaginyl ligases are different and
(1) the first asparaginyl ligase is VyPAL2 or a variant thereof and the second asparaginyl ligase is butelase-1 or a variant thereof; or
(2) the first asparaginyl ligase is butelase-1 or a variant thereof and the second asparaginyl ligase is VyPAL2 or a variant thereof

8. The method of claim 1, wherein the binding and ligation site for VyPAL2 or a variant thereof has the amino acid sequence (X)ₒNX³X⁴(X)ₚ, wherein X is any amino acid and o is an integer of at least 2, X³ is G or S, and X⁴ is a hydrophobic or aromatic amino acid, preferably selected from L, I, V, F, C, W, Y and M, preferably L or F, and p is 0 or an integer of 1 or more.

9. The method of claim 1, wherein the binding and ligation site for butelase-1 or a variant thereof has the amino acid sequence (X)ₒNX³X⁴(X)ₚ, wherein X is any amino acid and o is an integer of at least 2, X³ is H, and X⁴ is a hydrophobic or aromatic amino acid, preferably selected from L, I, V, F, C, W, Y and M, preferably V, and p is 0 or an integer of 1 or more.

10. The method of claim 1, wherein
(1) the first asparaginyl ligase is VyPAL2 or a variant thereof and said binding and ligation site for VyPAL2 or variant thereof is located at the C-terminus of the second (poly)peptide and the N-terminus of the first (poly)peptide has the amino acid sequence X¹F(X)_{q}, wherein X¹ can be any amino acid with the exception of Pro, X can be any amino acid, and q is 0 or an integer of 1 or more, preferably an integer of 1 or more, more preferably of at least 3, even more preferably of at least 5; or
(2) the first asparaginyl ligase is VyPAL2 or a variant thereof and said binding and ligation site for VyPAL2 or variant thereof is located at the C-terminus of the first (poly)peptide and the N-terminus of the second (poly)peptide has the amino acid sequence X¹F(X)_{q}, wherein X¹ can be any amino acid with the exception of Pro, X can be any amino acid, and q is 0 or an integer of 1 or more, preferably an integer of 1 or more, more preferably of at least 3, even more preferably of at least 5; or
(3) the second asparaginyl ligase is VyPAL2 or a variant thereof and said binding and ligation site for VyPAL2 or variant thereof is located at the C-terminus of the third (poly)peptide and the N-terminus of the first (poly)peptide has the amino acid sequence X¹F(X)_{q}, wherein X¹ can be any amino acid with the exception of Pro, X can be any amino acid, and q is 0 or an integer of 1 or more, preferably an integer of 1 or more, more preferably of at least 3, even more preferably of at least 5; or
(4) the second asparaginyl ligase is VyPAL2 or a variant thereof and said binding and ligation site for VyPAL2 or variant thereof is located at the C-terminus of the first (poly)peptide and the N-terminus of the third (poly)peptide has the amino acid sequence X¹F(X)_{q}, wherein X¹ can be any amino acid with the exception of Pro, X can be any amino acid, and q is 0 or an integer of 1 or more, preferably an integer of 1 or more, more preferably of at least 3, even more preferably of at least 5.

11. The method of claim 1, wherein
(1) the first asparaginyl ligase is butelase-1 or a variant thereof and said binding and ligation site for butelase-1 or variant thereof is located at the C-terminus of the second (poly)peptide and the N-terminus of the first (poly)peptide has the amino acid sequence X¹X²(X)_{q} with X¹ being G or H and X² being L, V or I, X being any amino acid, and q being 0 or an integer of 1 or more, preferably an integer of 1 or more, more preferably of at least 3, even more preferably of at least 5; or
(2) the first asparaginyl ligase is butelase-1 or a variant thereof and said binding and ligation site for butelase-1 or variant thereof is located at the C-terminus of the first (poly)peptide and the N-terminus of the second (poly)peptide has the amino acid sequence X¹X²(X)_{q} with X¹ being G or H and X² being L, V or I, X being any amino acid, and q being 0 or an integer of 1 or more, preferably an integer of 1 or more, more preferably of at least 3, even more preferably of at least 5; or
(3) the second asparaginyl ligase is butelase-1 or a variant thereof and said binding and ligation site for butelase-1 or variant thereof is located at the C-terminus of the third (poly)peptide and the N-terminus of the first (poly)peptide has the amino acid sequence X¹X²(X)_{q} with X¹ being G or H and X² being L, V or I, X being any amino acid, and q being 0 or an integer of 1 or more, preferably an integer of 1 or more, more preferably of at least 3, even more preferably of at least 5; or
(4) the second asparaginyl ligase is butelase-1 or a variant thereof and said binding and ligation site for butelase-1 or variant thereof is located at the C-terminus of the first (poly)peptide and the N-terminus of the third (poly)peptide has the amino acid sequence X¹X²(X)_{q} with X¹ being G or H and X² being L, V or I, X being any amino acid, and q being 0 or an integer of 1 or more, preferably an integer of 1 or more, more preferably of at least 3, even more preferably of at least 5.

12. The method of claim 1, wherein steps (i) and (ii) are carried out at a first and a second pH-value that are different from each other.

13. The method of claim 12, wherein the first and the second asparaginyl ligase are identical and the asparaginyl ligase has pH-dependent activity and specificity.

14. The method of claim 12, wherein
(1) the binding and ligation site for an asparaginyl ligase at the C-terminus of the first (poly)peptide is preferably bound and ligated by the asparaginyl ligase at the first pH value and the binding and ligation site for an asparaginyl ligase at the C-terminus of either the second or third (poly)peptide is preferably bound and ligated by the asparaginyl ligase at the second pH value; or
(2) the binding and ligation site for an asparaginyl ligase at the C-terminus of the first (poly)peptide is preferably bound and ligated by the asparaginyl ligase at the second pH value and the binding and ligation site for an asparaginyl ligase at the C-terminus of either the second or third (poly)peptide is preferably bound and ligated by the asparaginyl ligase at the first pH value.

15. The method of claim 12, wherein
(1) the first pH value is a pH of about 6.0 or lower, preferably a pH in the range of 4.5-6.0, and the second pH value is a pH of about 6.5 or higher, preferably a pH in the range of 6.5-7.4; or
(2) the second pH value is a pH of about 6.0 or lower, preferably a pH in the range of 4.5-6.0, and the first pH value is a pH of about 6.5 or higher, preferably a pH in the range of 6-5-7.4.

16. The method of claim 12, wherein the asparaginyl ligase is VyPAL2 comprising or consisting of the amino acid sequence set forth in SEQ ID NO:1 or a variant thereof that has an amino acid sequence that has at least 80% sequence identity to the amino acid sequence set forth in SEQ ID NO:1 over its entire length.

17. Method for (poly)peptide tandem ligation, the method comprising the steps of:
(i) contacting a first (poly)peptide (A) having at its C-terminus a binding and ligation site for an asparaginyl ligase with a second (poly)peptide (B) to be ligated to said first (poly)peptide and a first asparaginyl ligase (C) under conditions that allow ligation of the second (poly)peptide to the C- or N-terminus of the first (poly)peptide to yield a modified first (poly)peptide;
(ii) contacting the modified first (poly)peptide obtained in step (i) with a third (poly)peptide (D) to be ligated to said modified first (poly)peptide and a second asparaginyl ligase (E) under conditions that allow ligation of the third (poly)peptide to the C- or N-terminus of the first (poly)peptide to yield a dually modified first (poly)peptide;
wherein steps (i) and (ii) are carried out at a first and a second pH-value that are different from each other, wherein the first pH value is a pH of about 6.0 or lower, preferably a pH in the range of 4.5-6.0, and the second pH value is a pH of about 6.5 or higher, preferably a pH in the range of 6.5-7.4, wherein the first and second asparaginyl ligases are different and wherein the asparaginyl ligase used at a pH of about 6 or lower is OaAEP 1b comprising or consisting of the amino acid sequence set forth in SEQ ID NO:44 or a variant thereof that has an amino acid sequence that has at least 80% sequence identity to the amino acid sequence set forth in SEQ ID NO:44 over its entire length and the asparaginyl ligase used at a pH of about 6.5 or higher is (i) VyPAL2 comprising or consisting of the amino acid sequence set forth in SEQ ID NO:1 or a variant thereof that has an amino acid sequence that has at least 80% sequence identity to the amino acid sequence set forth in SEQ ID NO:1 over its entire length or (ii) butelase-1 comprising or consisting of the amino acid sequence set forth in SEQ ID NO:2 and variants thereof that share at least 80% sequence identity with the amino acid sequence set forth in SEQ ID NO:2 over their entire length.

18. The method of claim 12, wherein
(1) the binding and ligation site for an asparaginyl ligase at the C-terminus of the first (poly)peptide has the amino acid sequence (X)ₒDX³X⁴ (X)ₚ, wherein X is any amino acid, o is an integer of at least 2, X³ is an amino acid selected from A, C, F, G, H, K, N, Q, R, S, Y, preferably G, S, N, Q and R, more preferably G or S, and X⁴ is a hydrophobic or aromatic amino acid, preferably selected from L, I, V, F, C, W, Y and M, preferably L, I, and F, more preferably L or F, and p is 0 or an integer of 1 or more; and the binding and ligation site for an asparaginyl ligase at the C-terminus of the second or third (poly)peptide has the amino acid sequence (X)ₒNX³ X⁴ (X)ₚ, wherein X is any amino acid and o is an integer of at least 2, X³ is G or S, and X⁴ is a hydrophobic or aromatic amino acid, preferably selected from L, I, V, F, C, W, Y and M, preferably L or F, and p is 0 or an integer of 1 or more; or
(2) the binding and ligation site for an asparaginyl ligase at the C-terminus of the second or third (poly)peptide has the amino acid sequence (X)ₒDX³X⁴(X)ₚ, wherein X is any amino acid, o is an integer of at least 2, X³ is an amino acid selected from A, C, F, G, H, K, N, Q, R, S, Y, preferably G, S, N, Q and R, more preferably G or S, and X⁴ is a hydrophobic or aromatic amino acid, preferably selected from L, I, V, F, C, W, Y and M, preferably L, I, and F, more preferably L or F, and p is 0 or an integer of 1 or more; and the binding an ligation site for an asparaginyl ligase at the C-terminus of the first (poly)peptide has the amino acid sequence (X)ₒNX³X⁴ (X)ₚ, wherein X is any amino acid and o is an integer of at least 2, X³ is G or S, and X⁴ is a hydrophobic or aromatic amino acid, preferably selected from L, I, V, F, C, W, Y and M, preferably L or F, and p is 0 or an integer of 1 or more.

19. The method of claim 18, wherein the binding and ligation site having the amino acid sequence (X)ₒDX³X⁴ (X)ₚ is preferably bound to by the asparaginyl ligase at a pH of about 6.0 or lower, preferably a pH in the range of 4.5-6.0, and the binding and ligation site having the amino acid sequence (X)ₒNX³X⁴ (X)ₚ is preferably bound to by the asparaginyl ligase at a pH of about 6.5 or higher, preferably a pH in the range of 6.5 to 7.4.

20. Method for (poly)peptide cyclization, the method comprising the steps of:
(i) contacting (A) a first (poly)peptide having (i) at its C-terminus a binding and ligation site for an asparaginyl ligase with (B) a second (poly)peptide to be ligated to said first (poly)peptide having at its C-terminus a binding an ligation site for an asparaginyl ligase and (C) a first asparaginyl ligase under conditions that allow ligation of the second (poly)peptide to the C- or N-terminus of the first (poly)peptide to yield a modified first (poly)peptide;
(ii) contacting the modified first (poly)peptide obtained in step (i) with (E) a second asparaginyl ligase under conditions that allow ligation of the C-terminus of the modified first (poly)peptide to its N-terminus to yield a cyclized first (poly)peptide;
wherein the first or second asparaginyl ligase is selected from VyPAL2 comprising or consisting of the amino acid sequence set forth in SEQ ID NO:1 and variants thereof that share at least 80% sequence identity with the amino acid sequence set forth in SEQ ID NO:1 over their entire length, and the other asparaginyl ligase is selected from butelase-1 comprising or consisting of the amino acid sequence set forth in SEQ ID NO:2 and variants thereof that share at least 80% sequence identity with the amino acid sequence set forth in SEQ ID NO:2 over their entire length.

21. The method of claim 1, wherein at least one of the (poly)peptides to be ligated is further conjugated to an organic moiety.

22. The method of claim 21, wherein the organic moiety is a pharmaceutically active agent or a detectable marker.
